SEQUENCE LISTING (213 sequences)

SEQ ID NO: 1 - protein, 5 aa, Artificial Sequence
  Synthetic
SDYMH

SEQ ID NO: 2 - protein, 5 aa, Artificial Sequence
  Synthetic
GYYMH

SEQ ID NO: 3 - protein, 5 aa, Artificial Sequence
  Synthetic
GYYMH

SEQ ID NO: 4 - protein, 5 aa, Artificial Sequence
  Synthetic
GYYIH

SEQ ID NO: 5 - protein, 5 aa, Artificial Sequence
  Synthetic
THYMH

SEQ ID NO: 6 - protein, 5 aa, Artificial Sequence
  Synthetic
SHDIN

SEQ ID NO: 7 - protein, 5 aa, Artificial Sequence
  Synthetic
DHYLH

SEQ ID NO: 8 - protein, 5 aa, Artificial Sequence
  Synthetic
NYYMH

SEQ ID NO: 9 - protein, 5 aa, Artificial Sequence
  Synthetic
AYYVH

SEQ ID NO: 10 - protein, 5 aa, Artificial Sequence
  Synthetic
RHYVH

SEQ ID NO: 11 - protein, 4 aa, Artificial Sequence
  Synthetic
NYIH

SEQ ID NO: 12 - protein, 5 aa, Artificial Sequence
  Synthetic
NHYVH

SEQ ID NO: 13 - protein, 5 aa, Artificial Sequence
  Synthetic
SHYMH

SEQ ID NO: 14 - protein, 5 aa, Artificial Sequence
  Synthetic
RHLLH

SEQ ID NO: 15 - protein, 17 aa, Artificial Sequence
  Synthetic
WMSPYNGITGYAQKFQG

SEQ ID NO: 16 - protein, 17 aa, Artificial Sequence
  Synthetic
WMSPSSGITGYAQKFQG

SEQ ID NO: 17 - protein, 17 aa, Artificial Sequence
  Synthetic
WMTTNSGITGYAQKFQG

SEQ ID NO: 18 - protein, 17 aa, Artificial Sequence
  Synthetic
GIIPIFGTASYAQKFQG

SEQ ID NO: 19 - protein, 17 aa, Artificial Sequence
  Synthetic
WMNPNSGHAGSAQKFQG

SEQ ID NO: 20 - protein, 17 aa, Artificial Sequence
  Synthetic
WMNPNSGNTGYAQKFQG

SEQ ID NO: 21 - protein, 17 aa, Artificial Sequence
  Synthetic
WMNPNSGNTGYSQKFQG

SEQ ID NO: 22 - protein, 17 aa, Artificial Sequence
  Synthetic
WMNPNIGNTGYAQKFQG

SEQ ID NO: 23 - protein, 17 aa, Artificial Sequence
  Synthetic
WMNPNGGTTGYAQNFQG

SEQ ID NO: 24 - protein, 17 aa, Artificial Sequence
  Synthetic
WMNPNRGITDSAQKFQG

SEQ ID NO: 25 - protein, 17 aa, Artificial Sequence
  Synthetic
WMNPNSGSAGYAQKFQG

SEQ ID NO: 26 - protein, 17 aa, Artificial Sequence
  Synthetic
WIHPRSGATGYAPKFQG

SEQ ID NO: 27 - protein, 17 aa, Artificial Sequence
  Synthetic
WISPRSGVTSYAQKFQG

SEQ ID NO: 28 - protein, 17 aa, Artificial Sequence
  Synthetic
WMDPNSGNTGYAQKFQG

SEQ ID NO: 29 - protein, 17 aa, Artificial Sequence
  Synthetic
WMNPTGGITGYAQKFQG

SEQ ID NO: 30 - protein, 17 aa, Artificial Sequence
  Synthetic
WMNPNSGHTGNAQKFQG

SEQ ID NO: 31 - protein, 17 aa, Artificial Sequence
  Synthetic
WVSPIHGLTGYAPRFQG

SEQ ID NO: 32 - protein, 9 aa, Artificial Sequence
  Synthetic
DRFSGSYDY

SEQ ID NO: 33 - protein, 7 aa, Artificial Sequence
  Synthetic
DRGWFDP

SEQ ID NO: 34 - protein, 9 aa, Artificial Sequence
  Synthetic
EGYSSGLDY

SEQ ID NO: 35 - protein, 11 aa, Artificial Sequence
  Synthetic
DGRFWSGYPDY

SEQ ID NO: 36 - protein, 10 aa, Artificial Sequence
  Synthetic
ESIAVAGYDY

SEQ ID NO: 37 - protein, 10 aa, Artificial Sequence
  Synthetic
DRWYMGSADY

SEQ ID NO: 38 - protein, 10 aa, Artificial Sequence
  Synthetic
DDWGGDWFDP

SEQ ID NO: 39 - protein, 10 aa, Artificial Sequence
  Synthetic
ERLSVAGFDY

SEQ ID NO: 40 - protein, 10 aa, Artificial Sequence
  Synthetic
EPLQLGGFDY

SEQ ID NO: 41 - protein, 10 aa, Artificial Sequence
  Synthetic
EGFGPNAFDI

SEQ ID NO: 42 - protein, 10 aa, Artificial Sequence
  Synthetic
DSWYGDWFDP

SEQ ID NO: 43 - protein, 9 aa, Artificial Sequence
  Synthetic
EVIEVGMDV

SEQ ID NO: 44 - protein, 9 aa, Artificial Sequence
  Synthetic
EAWFGELST

SEQ ID NO: 45 - protein, 9 aa, Artificial Sequence
  Synthetic
EAYVAAFDI

SEQ ID NO: 46 - protein, 9 aa, Artificial Sequence
  Synthetic
ERGYNAFDY

SEQ ID NO: 47 - protein, 8 aa, Artificial Sequence
  Synthetic
DSVFGLDY

SEQ ID NO: 48 - protein, 10 aa, Artificial Sequence
  Synthetic
DLDYVRAFDI

SEQ ID NO: 49 - protein, 9 aa, Artificial Sequence
  Synthetic
ESWGGYFDL

SEQ ID NO: 50 - protein, 9 aa, Artificial Sequence
  Synthetic
DRTTYAFDI

SEQ ID NO: 51 - protein, 9 aa, Artificial Sequence
  Synthetic
GNWVDAFDI

SEQ ID NO: 52 - protein, 11 aa, Artificial Sequence
  Synthetic
VHGSGSDGMDV

SEQ ID NO: 53 - protein, 11 aa, Artificial Sequence
  Synthetic
RASQSISSWLA

SEQ ID NO: 54 - protein, 11 aa, Artificial Sequence
  Synthetic
RASQSVGTWLA

SEQ ID NO: 55 - protein, 11 aa, Artificial Sequence
  Synthetic
RASQGISNYLA

SEQ ID NO: 56 - protein, 11 aa, Artificial Sequence
  Synthetic
RASQSISTWLA

SEQ ID NO: 57 - protein, 11 aa, Artificial Sequence
  Synthetic
QASQDISNHLN

SEQ ID NO: 58 - protein, 11 aa, Artificial Sequence
  Synthetic
RASQGISSWLA

SEQ ID NO: 59 - protein, 11 aa, Artificial Sequence
  Synthetic
RASESISSWLA

SEQ ID NO: 60 - protein, 11 aa, Artificial Sequence
  Synthetic
RASQSVGSWLA

SEQ ID NO: 61 - protein, 11 aa, Artificial Sequence
  Synthetic
RASQNISNFLN

SEQ ID NO: 62 - protein, 12 aa, Artificial Sequence
  Synthetic
RASQSLSSSYLA

SEQ ID NO: 63 - protein, 11 aa, Artificial Sequence
  Synthetic
RASQSISSYLN

SEQ ID NO: 64 - protein, 11 aa, Artificial Sequence
  Synthetic
RASQSISRWLA

SEQ ID NO: 65 - protein, 11 aa, Artificial Sequence
  Synthetic
RDSHSITTWLA

SEQ ID NO: 66 - protein, 11 aa, Artificial Sequence
  Synthetic
RASQVIRNDLA

SEQ ID NO: 67 - protein, 11 aa, Artificial Sequence
  Synthetic
RASQSISRYLN

SEQ ID NO: 68 - protein, 7 aa, Artificial Sequence
  Synthetic
AASSLQS

SEQ ID NO: 69 - protein, 7 aa, Artificial Sequence
  Synthetic
AASTLEN

SEQ ID NO: 70 - protein, 7 aa, Artificial Sequence
  Synthetic
RASNLES

SEQ ID NO: 71 - protein, 7 aa, Artificial Sequence
  Synthetic
AASTLQR

SEQ ID NO: 72 - protein, 7 aa, Artificial Sequence
  Synthetic
AASTLQS

SEQ ID NO: 73 - protein, 7 aa, Artificial Sequence
  Synthetic
GASNLQR

SEQ ID NO: 74 - protein, 7 aa, Artificial Sequence
  Synthetic
AASNLQS

SEQ ID NO: 75 - protein, 7 aa, Artificial Sequence
  Synthetic
GASSLQS

SEQ ID NO: 76 - protein, 7 aa, Artificial Sequence
  Synthetic
AASHLQS

SEQ ID NO: 77 - protein, 7 aa, Artificial Sequence
  Synthetic
GASTRAT

SEQ ID NO: 78 - protein, 7 aa, Artificial Sequence
  Synthetic
DSSSLQT

SEQ ID NO: 79 - protein, 7 aa, Artificial Sequence
  Synthetic
AASNLES

SEQ ID NO: 80 - protein, 9 aa, Artificial Sequence
  Synthetic
QQSYSTPYT

SEQ ID NO: 81 - protein, 9 aa, Artificial Sequence
  Synthetic
QQSFSTPYT

SEQ ID NO: 82 - protein, 9 aa, Artificial Sequence
  Synthetic
QQSYSTPLT

SEQ ID NO: 83 - protein, 9 aa, Artificial Sequence
  Synthetic
QQSYSTPFT

SEQ ID NO: 84 - protein, 9 aa, Artificial Sequence
  Synthetic
QQSYSTPIT

SEQ ID NO: 85 - protein, 9 aa, Artificial Sequence
  Synthetic
QQYYSTPYT

SEQ ID NO: 86 - protein, 9 aa, Artificial Sequence
  Synthetic
QQSYSLPYT

SEQ ID NO: 87 - protein, 9 aa, Artificial Sequence
  Synthetic
HQYFTTPLT

SEQ ID NO: 88 - protein, 9 aa, Artificial Sequence
  Synthetic
QQSYSMPYT

SEQ ID NO: 89 - protein, 9 aa, Artificial Sequence
  Synthetic
QQSYSTPVT

SEQ ID NO: 90 - protein, 9 aa, Artificial Sequence
  Synthetic
QHFYNTQYT

SEQ ID NO: 91 - protein, 10 aa, Artificial Sequence
  Synthetic
QQSLQYPSHF

SEQ ID NO: 92 - protein, 30 aa, Artificial Sequence
  Synthetic
QVQLVQSGAEVKKPGASVKVSCKASGYTFS

SEQ ID NO: 93 - protein, 30 aa, Artificial Sequence
  Synthetic
QVQLVQSGAEVKKPGASVKVSCKASGYTFT

SEQ ID NO: 94 - protein, 30 aa, Artificial Sequence
  Synthetic
QVQLVQSGAEVKKPGSSVKVSCKASGYTFT

SEQ ID NO: 95 - protein, 30 aa, Artificial Sequence
  Synthetic
QVQLVQSGAEVKEPGASVKVSCKASGYTFT

SEQ ID NO: 96 - protein, 30 aa, Artificial Sequence
  Synthetic
QVQLVQSGAEVKKPGASVKVSCKASGYNFS

SEQ ID NO: 97 - protein, 30 aa, Artificial Sequence
  Synthetic
QVQLVQSGAEVKKPGASVKVSCKASGYTLP

SEQ ID NO: 98 - protein, 30 aa, Artificial Sequence
  Synthetic
QVQLVQSGAEVKKPGASVKVSCKASGYSFT

SEQ ID NO: 99 - protein, 30 aa, Artificial Sequence
  Synthetic
QVQLVQSGAEVKKPGASVKVSCKASGYTFI

SEQ ID NO: 100 - protein, 14 aa, Artificial Sequence
  Synthetic
WVRQAPGQGLEWMG

SEQ ID NO: 101 - protein, 14 aa, Artificial Sequence
  Synthetic
WMRQAPGQGLEWIG

SEQ ID NO: 102 - protein, 32 aa, Artificial Sequence
  Synthetic
RVTMTRDTSTSTVYMELSSLRSEDTAVYYCAR

SEQ ID NO: 103 - protein, 32 aa, Artificial Sequence
  Synthetic
RVTITADESTSTAYMELSSLRSEDTAVYYCAR

SEQ ID NO: 104 - protein, 11 aa, Artificial Sequence
  Synthetic
WGQGTLVTVSS

SEQ ID NO: 105 - protein, 11 aa, Artificial Sequence
  Synthetic
WGQGTTVTVSS

SEQ ID NO: 106 - protein, 11 aa, Artificial Sequence
  Synthetic
WGRGTLVTVSS

SEQ ID NO: 107 - protein, 11 aa, Artificial Sequence
  Synthetic
WGQGTMVTVSS

SEQ ID NO: 108 - protein, 23 aa, Artificial Sequence
  Synthetic
DIQMTQSPSSLSASVGDRVTITC

SEQ ID NO: 109 - protein, 23 aa, Artificial Sequence
  Synthetic
EIVMTQSPATLSVSPGERATLSC

SEQ ID NO: 110 - protein, 23 aa, Artificial Sequence
  Synthetic
DIQITHSPSSLSASVGYRLTITC

SEQ ID NO: 111 - protein, 15 aa, Artificial Sequence
  Synthetic
WYQQKPGKAPKLLIY

SEQ ID NO: 112 - protein, 15 aa, Artificial Sequence
  Synthetic
WYQQKPGQAPRLLIY

SEQ ID NO: 113 - protein, 15 aa, Artificial Sequence
  Synthetic
WYHQKPWNAPKLMIY

SEQ ID NO: 114 - protein, 32 aa, Artificial Sequence
  Synthetic
GVPSRFSGSGSGTDFTLTISSLQPEDFATYYC

SEQ ID NO: 115 - protein, 32 aa, Artificial Sequence
  Synthetic
GIPARFSGSGSGTEFTLTISSLQSEDFAVYYC

SEQ ID NO: 116 - protein, 32 aa, Artificial Sequence
  Synthetic
GVPSRFSGSGSGTYFTLTISSLQPEDFATYYC

SEQ ID NO: 117 - protein, 32 aa, Artificial Sequence
  Synthetic
GVPSRFSGSGSGTDFTLTISSLQPEDFAPYYC

SEQ ID NO: 118 - protein, 10 aa, Artificial Sequence
  Synthetic
FGGGTKVEIK

SEQ ID NO: 119 - protein, 10 aa, Artificial Sequence
  Synthetic
FGQGTKLEIK

SEQ ID NO: 120 - protein, 10 aa, Artificial Sequence
  Synthetic
FGQGTKVEIK

SEQ ID NO: 121 - protein, 10 aa, Artificial Sequence
  Synthetic
FGQGTRLEIK

SEQ ID NO: 122 - protein, 10 aa, Artificial Sequence
  Synthetic
FGPGTKVDIK

SEQ ID NO: 123 - protein, 118 aa, Artificial Sequence
  Synthetic
QVQLVQSGAEVKKPGASVKVSCKASGYTFSSDYMHWVRQAPGQGLEWMGWMSPYNGITGYAQKFQGRVTMTRDTSTSTVYMELSSLRSEDTAVYYCARDRFSGSYDYWGQGTLVTVSS

SEQ ID NO: 124 - protein, 116 aa, Artificial Sequence
  Synthetic
QVQLVQSGAEVKKPGASVKVSCKASGYTFTGYYMHWVRQAPGQGLEWMGWMSPSSGITGYAQKFQGRVTMTRDTSTSTVYMELSSLRSEDTAVYYCARDRGWFDPWGQGTLVTVSS

SEQ ID NO: 125 - protein, 118 aa, Artificial Sequence
  Synthetic
QVQLVQSGAEVKKPGASVKVSCKASGYTFSSYYMHWVRQAPGQGLEWMGWMTTNSGITGYAQKFQGRVTMTRDTSTSTVYMELSSLRSEDTAVYYCAREGYSSGLDYWGQGTLVTVSS

SEQ ID NO: 126 - protein, 120 aa, Artificial Sequence
  Synthetic
QVQLVQSGAEVKKPGSSVKVSCKASGYTFTGYYIHWVRQAPGQGLEWMGGIIPIFGTASYAQKFQGRVTITADESTSTAYMELSSLRSEDTAVYYCARDGRFWSGYPDYWGQGTLVTVSS

SEQ ID NO: 127 - protein, 119 aa, Artificial Sequence
  Synthetic
QVQLVQSGAEVKKPGASVKVSCKASGYTFSTHYMHWVRQAPGQGLEWMGWMNPNSGHAGSAQKFQGRVTMTRDTSTSTVYMELSSLRSEDTAVYYCARESIAVAGYDYWGQGTLVTVSS

SEQ ID NO: 128 - protein, 119 aa, Artificial Sequence
  Synthetic
QVQLVQSGAEVKKPGASVKVSCKASGYTFTSHDINWVRQAPGQGLEWMGWMNPNSGNTGYAQKFQGRVTMTRDTSTSTVYMELSSLRSEDTAVYYCARDRWYMGSADYWGQGTLVTVSS

SEQ ID NO: 129 - protein, 119 aa, Artificial Sequence
  Synthetic
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCARDDWGGDWFDPWGQGTLVTVSS

SEQ ID NO: 130 - protein, 119 aa, Artificial Sequence
  Synthetic
QVQLVQSGAEVKKPGASVKVSCKASGYTFSTHYMHWVRQAPGQGLEWMGWMNPNSGNTGYSQKFQGRVTMTRDTSTSTVYMELSSLRSEDTAVYYCARERLSVAGFDYWGQGTLVTVSS

SEQ ID NO: 131 - protein, 119 aa, Artificial Sequence
  Synthetic
QVQLVQSGAEVKEPGASVKVSCKASGYTFTDHYLHWVRQAPGQGLEWMGWMNPNIGNTGYAQKFQGRVTMTRDTSTSTVYMELSSLRSEDTAVYYCAREPLQLGGFDYWGQGTLVTVSS

SEQ ID NO: 132 - protein, 119 aa, Artificial Sequence
  Synthetic
QVQLVQSGAEVKKPGASVKVSCKASGYTFTGYYMHWVRQAPGQGLEWMGWMNPNGGTTGYAQNFQGRVTMTRDTSTSTVYMELSSLRSEDTAVYYCAREGFGPNAFDIWGQGTTVTVSS

SEQ ID NO: 133 - protein, 119 aa, Artificial Sequence
  Synthetic
QVQLVQSGAEVKKPGASVKVSCKASGYTFTGYYMHWVRQAPGQGLEWMGWMNPNSGNTGYAQKFQGRVTMTRDTSTSTVYMELSSLRSEDTAVYYCARDSWYGDWFDPWGQGTLVTVSS

SEQ ID NO: 134 - protein, 118 aa, Artificial Sequence
  Synthetic
QVQLVQSGAEVKKPGASVKVSCKASGYTFTGYYMHWMRQAPGQGLEWIGWMSPYNGITGYAQKFQGRVTMTRDTSTSTVYMELSSLRSEDTAVYYCAREVIEVGMDVWGQGTTVTVSS

SEQ ID NO: 135 - protein, 118 aa, Artificial Sequence
  Synthetic
QVQLVQSGAEVKKPGASVKVSCKASGYTFTNYYMHWVRQAPGQGLEWMGWMSPSSGITGYAQKFQGRVTMTRDTSTSTVYMELSSLRSEDTAVYYCAREAWFGELSTWGQGTLVTVSS

SEQ ID NO: 136 - protein, 118 aa, Artificial Sequence
  Synthetic
QVQLVQSGAEVKKPGASVKVSCKASGYNFSAYYVHWVRQAPGQGLEWMGWMNPNRGITDSAQKFQGRVTMTRDTSTSTVYMELSSLRSEDTAVYYCAREAYVAAFDIWGQGTTVTVSS

SEQ ID NO: 137 - protein, 118 aa, Artificial Sequence
  Synthetic
QVQLVQSGAEVKKPGASVKVSCKASGYTFTRHYVHWVRQAPGQGLEWMGWMNPNSGSAGYAQKFQGRVTMTRDTSTSTVYMELSSLRSEDTAVYYCARERGYNAFDYWGQGTLVTVSS

SEQ ID NO: 138 - protein, 116 aa, Artificial Sequence
  Synthetic
QVQLVQSGAEVKKPGASVKVSCKASGYTLPNYIHWVRQAPGQGLEWMGWIHPRSGATGYAPKFQGRVTMTRDTSTSTVYMELSSLRSEDTAVYYCARDSVFGLDYWGQGTLVTVSS

SEQ ID NO: 139 - protein, 119 aa, Artificial Sequence
  Synthetic
QVQLVQSGAEVKKPGASVKVSCKASGYTFTSYYMHWVRQAPGQGLEWMGWISPRSGVTSYAQKFQGRVTMTRDTSTSTVYMELSSLRSEDTAVYYCARDLDYVRAFDIWGQGTTVTVSS

SEQ ID NO: 140 - protein, 118 aa, Artificial Sequence
  Synthetic
QVQLVQSGAEVKKPGASVKVSCKASGYSFTSYYMHWVRQAPGQGLEWMGWMDPNSGNTGYAQKFQGRVTMTRDTSTSTVYMELSSLRSEDTAVYYCARESWGGYFDLWGRGTLVTVSS

SEQ ID NO: 141 - protein, 118 aa, Artificial Sequence
  Synthetic
QVQLVQSGAEVKKPGASVKVSCKASGYTFINHYVHWVRQAPGQGLEWMGWMNPTGGITGYAQKFQGRVTMTRDTSTSTVYMELSSLRSEDTAVYYCARDRTTYAFDIWGQGTMVTVSS

SEQ ID NO: 142 - protein, 118 aa, Artificial Sequence
  Synthetic
QVQLVQSGAEVKKPGASVKVSCKASGYTFTSHYMHWVRQAPGQGLEWMGWMNPNSGHTGNAQKFQGRVTMTRDTSTSTVYMELSSLRSEDTAVYYCARGNWVDAFDIWGQGTMVTVSS

SEQ ID NO: 143 - protein, 120 aa, Artificial Sequence
  Synthetic
QVQLVQSGAEVKKPGSSVKVSCKASGYTFTRHLLHWVRQAPGQGLEWMGWVSPIHGLTGYAPRFQGRVTITADESTSTAYMELSSLRSEDTAVYYCARVHGSGSDGMDVWGQGTTVTVSS

SEQ ID NO: 144 - protein, 107 aa, Artificial Sequence
  Synthetic
DIQMTQSPSSLSASVGDRVTITCRASQSISSWLAWYQQKPGKAPKLLIYAASSLQSGVPSRFSGSGSGTDFTLTISSLQPEDFATYYCQQSYSTPYTFGGGTKVEIK

SEQ ID NO: 145 - protein, 107 aa, Artificial Sequence
  Synthetic
DIQMTQSPSSLSASVGDRVTITCRASQSVGTWLAWYQQKPGKAPKLLIYAASTLENGVPSRFSGSGSGTDFTLTISSLQPEDFATYYCQQSFSTPYTFGGGTKVEIK

SEQ ID NO: 146 - protein, 107 aa, Artificial Sequence; identical to SEQ ID NO: 144
  Synthetic

SEQ ID NO: 147 - protein, 107 aa, Artificial Sequence
  Synthetic
DIQMTQSPSSLSASVGDRVTITCRASQGISNYLAWYQQKPGKAPKLLIYRASNLESGVPSRFSGSGSGTDFTLTISSLQPEDFATYYCQQSYSTPLTFGGGTKVEIK

SEQ ID NO: 148 - protein, 107 aa, Artificial Sequence
  Synthetic
DIQMTQSPSSLSASVGDRVTITCRASQSISSWLAWYQQKPGKAPKLLIYAASTLQRGVPSRFSGSGSGTDFTLTISSLQPEDFATYYCQQSYSTPYTFGQGTKLEIK

SEQ ID NO: 149 - protein, 107 aa, Artificial Sequence
  Synthetic
DIQMTQSPSSLSASVGDRVTITCRASQSISTWLAWYQQKPGKAPKLLIYAASTLQSGVPSRFSGSGSGTDFTLTISSLQPEDFATYYCQQSYSTPFTFGQGTKVEIK

SEQ ID NO: 150 - protein, 107 aa, Artificial Sequence
  Synthetic
DIQMTQSPSSLSASVGDRVTITCQASQDISNHLNWYQQKPGKAPKLLIYGASNLQRGVPSRFSGSGSGTDFTLTISSLQPEDFATYYCQQSYSTPITFGQGTRLEIK

SEQ ID NO: 151 - protein, 107 aa, Artificial Sequence
  Synthetic
DIQMTQSPSSLSASVGDRVTITCRASQGISSWLAWYQQKPGKAPKLLIYAASSLQSGVPSRFSGSGSGTDFTLTISSLQPEDFATYYCQQSYSTPYTFGQGTKVEIK

SEQ ID NO: 152 - protein, 107 aa, Artificial Sequence
  Synthetic
DIQMTQSPSSLSASVGDRVTITCRASESISSWLAWYQQKPGKAPKLLIYAASSLQSGVPSRFSGSGSGTDFTLTISSLQPEDFATYYCQQSYSTPLTFGQGTKLEIK

SEQ ID NO: 153 - protein, 107 aa, Artificial Sequence
  Synthetic
DIQMTQSPSSLSASVGDRVTITCRASQSISSWLAWYQQKPGKAPKLLIYAASNLQSGVPSRFSGSGSGTDFTLTISSLQPEDFATYYCQQYYSTPYTFGQGTKVEIK

SEQ ID NO: 154 - protein, 107 aa, Artificial Sequence
  Synthetic
DIQMTQSPSSLSASVGDRVTITCRASQSVGSWLAWYQQKPGKAPKLLIYGASSLQSGVPSRFSGSGSGTDFTLTISSLQPEDFATYYCQQSYSTPLTFGGGTKVEIK

SEQ ID NO: 155 - protein, 107 aa, Artificial Sequence
  Synthetic
DIQMTQSPSSLSASVGDRVTITCRASQSISSWLAWYQQKPGKAPKLLIYAASHLQSGVPSRFSGSGSGTDFTLTISSLQPEDFATYYCQQSYSTPYTFGQGTRLEIK

SEQ ID NO: 156 - protein, 107 aa, Artificial Sequence
  Synthetic
DIQMTQSPSSLSASVGDRVTITCRASQNISNFLNWYQQKPGKAPKLLIYAASSLQSGVPSRFSGSGSGTDFTLTISSLQPEDFATYYCQQSYSLPYTFGQGTKVEIK

SEQ ID NO: 157 - protein, 107 aa, Artificial Sequence
  Synthetic
DIQMTQSPSSLSASVGDRVTITCRASQGISSWLAWYQQKPGKAPKLLIYAASSLQSGVPSRFSGSGSGTDFTLTISSLQPEDFATYYCQQSYSTPLTFGGGTKVEIK

SEQ ID NO: 158 - protein, 108 aa, Artificial Sequence
  Synthetic
EIVMTQSPATLSVSPGERATLSCRASQSLSSSYLAWYQQKPGQAPRLLIYGASTRATGIPARFSGSGSGTEFTLTISSLQSEDFAVYYCHQYFTTPLTFGQGTRLEIK

SEQ ID NO: 159 - protein, 107 aa, Artificial Sequence
  Synthetic
DIQMTQSPSSLSASVGDRVTITCRASQSISSYLNWYQQKPGKAPKLLIYAASTLQSGVPSRFSGSGSGTDFTLTISSLQPEDFATYYCQQSYSMPYTFGQGTKLEIK

SEQ ID NO: 160 - protein, 107 aa, Artificial Sequence
  Synthetic
DIQMTQSPSSLSASVGDRVTITCRASQSISRWLAWYQQKPGKAPKLLIYAASSLQSGVPSRFSGSGSGTDFTLTISSLQPEDFATYYCQQSYSTPYTFGQGTKLEIK

SEQ ID NO: 161 - protein, 107 aa, Artificial Sequence
  Synthetic
DIQMTQSPSSLSASVGDRVTITCRASQSISSWLAWYQQKPGKAPKLLIYDSSSLQTGVPSRFSGSGSGTDFTLTISSLQPEDFATYYCQQSYSTPVTFGQGTKVEIK

SEQ ID NO: 162 - protein, 56 aa, Artificial Sequence
  Synthetic
DIQITHSPSSLSASVGYRLTITCRDSHSITTWLAWYHQKPWNAPKLMIYAASNLES

SEQ ID NO: 163 - protein, 108 aa, Artificial Sequence
  Synthetic
DIQMTQSPSSLSASVGDRVTITCRASQVIRNDLAWYQQKPGKAPKLLIYAASTLQSGVPSRFSGSGSGTDFTLTISSLQPEDFATYYCQQSLQYPSHFFGQGTKVEIK

SEQ ID NO: 164 - protein, 107 aa, Artificial Sequence
  Synthetic
DIQMTQSPSSLSASVGDRVTITCRASQSISRYLNWYQQKPGKAPKLLIYAASTLQSGVPSRFSGSGSGTDFTLTISSLQPEDFAPYYCQQSYSTPLTFGPGTKVDIK

SEQ ID NO: 165 - protein, 290 aa, Artificial Sequence
  Synthetic
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 166 - protein, 607 aa, Artificial Sequence
  Synthetic
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 167 - protein, 5 aa, Artificial Sequence
  Synthetic
NHYMH

SEQ ID NO: 168 - protein, 17 aa, Artificial Sequence
  Synthetic
WMNPNSGHTGYAQRFQG

SEQ ID NO: 169 - protein, 17 aa, Artificial Sequence
  Synthetic
WINPNSGNTGYAQKFQG

SEQ ID NO: 170 - protein, 17 aa, Artificial Sequence
  Synthetic
WINPNSGHTGYAQKFQG

SEQ ID NO: 171 - protein, 17 aa, Artificial Sequence
  Synthetic
WINPNSGNTGYSQKFQG

SEQ ID NO: 172 - protein, 17 aa, Artificial Sequence
  Synthetic
WMNPNSGHTGYAQKFQG

SEQ ID NO: 173 - protein, 17 aa, Artificial Sequence
  Synthetic
WINPNSGHTGYAQRFQG

SEQ ID NO: 174 - protein, 9 aa, Artificial Sequence
  Synthetic
EAVAGPMDV

SEQ ID NO: 175 - protein, 10 aa, Artificial Sequence
  Synthetic
  misc_feature (7)..(7): Xaa can be any naturally occurring amino acid
ERFLGGXMDV

SEQ ID NO: 176 - protein, 10 aa, Artificial Sequence
  Synthetic
  misc_feature (7)..(7): Xaa can be any naturally occurring amino acid
EAVAGPXMDV

SEQ ID NO: 177 - protein, 10 aa, Artificial Sequence
  Synthetic
DRWGGDYYSA

SEQ ID NO: 178 - protein, 11 aa, Artificial Sequence
  Synthetic
RASQGISSYLN

SEQ ID NO: 179 - protein, 11 aa, Artificial Sequence
  Synthetic
RASQSISSWLN

SEQ ID NO: 180 - protein, 11 aa, Artificial Sequence
  Synthetic
RASQSISSYLA

SEQ ID NO: 181 - protein, 11 aa, Artificial Sequence
  Synthetic
RASQGISSWLN

SEQ ID NO: 182 - protein, 11 aa, Artificial Sequence
  Synthetic
RASQGISSYLA

SEQ ID NO: 183 - protein, 30 aa, Artificial Sequence
  Synthetic
QVQLVQSGAEVKKPGASVKVSCKASGDTFT

SEQ ID NO: 184 - protein, 118 aa, Artificial Sequence
  Synthetic
QVQLVQSGAEVKKPGASVKVSCKASGDTFTNHYMHWVRQAPGQGLEWMGWMNPNSGHTGYAQRFQGRVTMTRDTSTSTVYMELSSLRSEDTAVYYCAREAVAGPMDVWGQGTTVTVSS

SEQ ID NO: 185 - protein, 119 aa, Artificial Sequence
  Synthetic
  misc_feature (105)..(105): Xaa can be any naturally occurring amino acid
QVQLVQSGAEVKKPGASVKVSCKASGYTFTSHYMHWVRQAPGQGLEWMGWINPNSGNTGYAQKFQGRVTMTRDTSTSTVYMELSSLRSEDTAVYYCARERFLGGXMDVWGQGTTVTVSS

SEQ ID NO: 186 - protein, 119 aa, Artificial Sequence
  Synthetic
  misc_feature (105)..(105): Xaa can be any naturally occurring amino acid
QVQLVQSGAEVKKPGASVKVSCKASGYTFTNHYMHWVRQAPGQGLEWMGWINPNSGHTGYAQKFQGRVTMTRDTSTSTVYMELSSLRSEDTAVYYCAREAVAGPXMDVWGQGTTVTVSS

SEQ ID NO: 187 - protein, 119 aa, Artificial Sequence
  Synthetic
QVQLVQSGAEVKKPGASVKVSCKASGYTFTNHYMHWVRQAPGQGLEWMGWINPNSGNTGYAQKFQGRVTMTRDTSTSTVYMELSSLRSEDTAVYYCARDRWGGDYYSAWGQGTLVTVSS

SEQ ID NO: 188 - protein, 119 aa, Artificial Sequence
  Synthetic
QVQLVQSGAEVKKPGASVKVSCKASGYTFSTHYMHWVRQAPGQGLEWMGWINPNSGNTGYSQKFQGRVTMTRDTSTSTVYMELSSLRSEDTAVYYCARERLSVAGFDYWGQGTLVTVSS

SEQ ID NO: 189 - protein, 118 aa, Artificial Sequence
  Synthetic
QVQLVQSGAEVKKPGASVKVSCKASGYTFTNHYMHWVRQAPGQGLEWMGWMNPNSGHTGYAQRFQGRVTMTRDTSTSTVYMELSSLRSEDTAVYYCAREAVAGPMDVWGQGTTVTVSS

SEQ ID NO: 190 - protein, 118 aa, Artificial Sequence
  Synthetic
QVQLVQSGAEVKKPGASVKVSCKASGDTFTNHYMHWVRQAPGQGLEWMGWMNPNSGHTGYAQKFQGRVTMTRDTSTSTVYMELSSLRSEDTAVYYCAREAVAGPMDVWGQGTTVTVSS

SEQ ID NO: 191 - protein, 118 aa, Artificial Sequence
  Synthetic
QVQLVQSGAEVKKPGASVKVSCKASGYTFTNHYMHWVRQAPGQGLEWMGWINPNSGHTGYAQRFQGRVTMTRDTSTSTVYMELSSLRSEDTAVYYCAREAVAGPMDVWGQGTTVTVSS

SEQ ID NO: 192 - protein, 118 aa, Artificial Sequence
  Synthetic
QVQLVQSGAEVKKPGASVKVSCKASGYTFTNHYMHWVRQAPGQGLEWMGWMNPNSGHTGYAQKFQGRVTMTRDTSTSTVYMELSSLRSEDTAVYYCAREAVAGPMDVWGQGTTVTVSS

SEQ ID NO: 193 - protein, 118 aa, Artificial Sequence
  Synthetic
QVQLVQSGAEVKKPGASVKVSCKASGDTFTNHYMHWVRQAPGQGLEWMGWINPNSGHTGYAQKFQGRVTMTRDTSTSTVYMELSSLRSEDTAVYYCAREAVAGPMDVWGQGTTVTVSS

SEQ ID NO: 194 - protein, 107 aa, Artificial Sequence [sequence omitted]

SEQ ID NO: 195 - protein, 107 aa, Artificial Sequence
  Synthetic
DIQMTQSPSSLSASVGDRVTITCRASQGISSYLNWYQQKPGKAPKLLIYAASSLQSGVPSRFSGSGSGTDFTLTISSLQPEDFATYYCQQSYSTPLTFGGGTKVEIK

SEQ ID NO: 196 - protein, 107 aa, Artificial Sequence
  Synthetic
DIQMTQSPSSLSASVGDRVTITCRASQSISSWLNWYQQKPGKAPKLLIYAASSLQSGVPSRFSGSGSGTDFTLTISSLQPEDFATYYCQQSYSTPLTFGGGTKVEIK

SEQ ID NO: 197 - protein, 107 aa, Artificial Sequence
  Synthetic
DIQMTQSPSSLSASVGDRVTITCRASQSISSYLAWYQQKPGKAPKLLIYAASSLQSGVPSRFSGSGSGTDFTLTISSLQPEDFATYYCQQSYSTPLTFGGGTKVEIK

SEQ ID NO: 198 - protein, 107 aa, Artificial Sequence
  Synthetic
DIQMTQSPSSLSASVGDRVTITCRASQGISSWLNWYQQKPGKAPKLLIYAASSLQSGVPSRFSGSGSGTDFTLTISSLQPEDFATYYCQQSYSTPLTFGGGTKVEIK

SEQ ID NO: 199 - protein, 107 aa, Artificial Sequence
  Synthetic
DIQMTQSPSSLSASVGDRVTITCRASQGISSYLAWYQQKPGKAPKLLIYAASSLQSGVPSRFSGSGSGTDFTLTISSLQPEDFATYYCQQSYSTPLTFGGGTKVEIK

SEQ ID NO: 200 - protein, 107 aa, Artificial Sequence
  Synthetic
DIQMTQSPSSLSASVGDRVTITCRASQSISSWLAWYQQKPGKAPKLLIYAASSLQSGVPSRFSGSGSGTDFTLTISSLQPEDFATYYCQQSYSTPLTFGGGTKVEIK

SEQ ID NO: 201 - protein, 107 aa, Artificial Sequence [sequence omitted]

SEQ ID NO: 202 - protein, 107 aa, Artificial Sequence
  Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 203 - protein, 17 aa, Artificial Sequence
  Synthetic
WITTNSGITGYAQKFQG

SEQ ID NO: 204 - protein, 17 aa, Artificial Sequence
  Synthetic
WINPNSGHAGSAQKFQG

SEQ ID NO: 205 - protein, 17 aa, Artificial Sequence
  Synthetic
WINPNSGTTGYAQNFQG

SEQ ID NO: 206 - protein, 17 aa, Artificial Sequence
  Synthetic
WINPNIGNTGYAQKFQG

SEQ ID NO: 207 - protein, 10 aa, Artificial Sequence
  Synthetic
DDWGGDWFDY

SEQ ID NO: 208 - protein, 10 aa, Artificial Sequence
  Synthetic
EPLQLGGFDY

SEQ ID NO: 209 - protein, 118 aa, Artificial Sequence
  Synthetic
QVQLVQSGAEVKKPGASVKVSCKASGYTFSSYYMHWVRQAPGQGLEWMGWITTNSGITGYAQKFQGRVTMTRDTSTSTVYMELSSLRSEDTAVYYCAREGYSSGLDYWGQGTLVTVSS

SEQ ID NO: 210 - protein, 119 aa, Artificial Sequence
  Synthetic
QVQLVQSGAEVKKPGASVKVSCKASGYTFSTHYMHWVRQAPGQGLEWMGWINPNSGHAGSAQKFQGRVTMTRDTSTSTVYMELSSLRSEDTAVYYCARESIAVAGYDYWGQGTLVTVSS

SEQ ID NO: 211 - protein, 119 aa, Artificial Sequence
  Synthetic
QVQLVQSGAEVKKPGASVKVSCKASGYTFTGYYMHWVRQAPGQGLEWMGWINPNSGTTGYAQNFQGRVTMTRDTSTSTVYMELSSLRSEDTAVYYCAREGFGPNAFDIWGQGTTVTVSS

SEQ ID NO: 212 - protein, 119 aa, Artificial Sequence
  Synthetic
QVQLVQSGAEVKKPGASVKVSCKASGYTFTSYYMHWVRQAPGQGLEWMGWINPNSGNTGYAQKFQGRVTMTRDTSTSTVYMELSSLRSEDTAVYYCARDDWGGDWFDYWGQGTLVTVSS

SEQ ID NO: 213 - protein, 119 aa, Artificial Sequence
  Synthetic
QVQLVQSGAEVKKPGASVKVSCKASGYTFTDHYLHWVRQAPGQGLEWMGWINPNIGNTGYAQKFQGRVTMTRDTSTSTVYMELSSLRSEDTAVYYCAREPLQLGGFDYWGQGTLVTVSS

(19) United States
(12) Patent Application Publication
(10) Pub. No.: US 2023/0069055 A1
(43) Pub. Date: Mar. 2, 2023

(54) ANTI-PD-L1 ANTIBODIES

(71) Applicant: BOLT BIOTHERAPEUTICS, INC., Redwood City, CA (US)

(72) Inventors: Michael N. ALONSO, Redwood City, CA (US); David DORNAN, Redwood City, CA (US); Karla HENNING, Redwood City, CA (US); Justin KENKEL, Redwood City, CA (US); Marcin KOWANETZ, Redwood City, CA (US); Heidi LEBLANC, Redwood City, CA (US); William MALLET, Redwood City, CA (US)

(73) Assignee: BOLT BIOTHERAPEUTICS, INC, Redwood City, CA (US)

(21) Appl. No.: 17/794,500

(22) PCT Filed: Jan. 21, 2021
(86) PCT No.: PCT/US2021/014347

§ 371 Date: Jul. 21, 2022

Related U.S. Application Data
(60) Provisional application No. 62/963,782, filed on Jan. 21, 2020.

(51) Int. Cl. C07K 16/28

(52) U.S. Cl. CPC C07K 16/2827; C07K 2317/77; C07K 2317/76; C07K 2317/92

(57) ABSTRACT

The invention relates to programmed death-ligand 1 (PD-L1) binding agents, nucleic acids comprising the inventive binding agents, vectors and cells comprising the inventive nucleic acids, and compositions thereof. The invention also relates to methods of producing the inventive binding agents, methods for treating a disease, disorder, or condition in a mammal, and methods for enhancing or reducing or inhibiting an immune response in a mammal.

### CROSS-REFERENCE TO RELATED APPLICATION

This patent application claims the benefit of U.S. Provisional Patent Application No. 62/963,771, filed Jan. 21, 2020, which is incorporated by reference in its entirety herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 110,747 Byte ASCII (Text) file named “751375_ST25.txt,” created on Jan. 21, 2021.

### BACKGROUND OF THE INVENTION

Programmed Death-Ligand 1 (PD-L1, cluster of differentiation 274, CD274, B7-homolog 1, or B7-H1) belongs to the B7 protein superfamily, and is a ligand of programmed cell death protein 1 (PD-1, PDCD1, cluster of differentiation 279, or CD279). PD-L1 can also interact with B7.1 (CD80) and such interaction is believed to inhibit T cell priming. The PD-L1/PD-1 axis plays a large role in suppressing the adaptive immune response. More specifically, it is believed that engagement of PD-L1 with its receptor, PD-1, delivers a signal that inhibits activation and proliferation of T-cells. Agents that bind to PD-L1 and prevent the ligand from binding to the PD-1 receptor prevent this immunosuppression, and can, therefore, enhance an immune response when desired, such as for the treatment of cancers or infections. PD-L1/PD-1 pathway also contributes to preventing autoimmunity and therefore agonistic agents against PD-L1 or agents that deliver immune inhibitory payloads may help treatment of autoimmune disorders.

Several antibodies targeting PD-L1 have been developed for the treatment of cancer, including atezolizumab (TECENTRIQ™), durvalumab (IMFINZI™), and avelumab (BAVENCIO™). Nevertheless, there continues to be a need for new PD-L1-binding agents, including agents that bind PD-L1 with high affinity and effectively prevent PD-L1/PD-1 signaling and agents that can deliver therapeutic payloads to PD-L1 expressing cells. In addition, there is a need for new PD-L1-binding agents to treat autoimmune disorders and infections.

### BRIEF SUMMARY OF THE INVENTION

Provided herein are PD-L1 binding agents comprising an immunoglobulin heavy chain variable region polypeptide and an immunoglobulin light chain variable region polypeptide. In some embodiments, the PD-L1 binding agents comprise an immunoglobulin heavy chain variable region of any one of SEQ ID NOs: 123-143, 184-193, or 209-213, or at least the CDRs thereof; and an immunoglobulin light chain variable region of any one of SEQ ID NOs: 144-164 or 194-202, or at least the CDRs thereof. In other embodiments, the PD-L1 binding agents comprise an immunoglobulin heavy chain variable region polypeptide with an amino acid sequence that is at least 90% identical to any one of SEQ ID NOs: 123-143, 184-193, or 209-213, and an immunoglobulin light chain variable region polypeptide with an amino acid sequence that is at least 90% identical to any one of SEQ ID NOs: 144-164 or 194-202. In yet other embodiments, the PD-L1 binding agent, the immunoglobulin heavy chain variable region polypeptide comprises a complementarity determining region 1 (HCDR1) comprising any one of SEQ ID NOs: 1-14 or 167, a complementarity determining region 2 (HCDR2) comprising any one of SEQ ID NOs: 15-31, 168-173, or 203-206, and a complementarity determining region 3 (HCDR3) comprising any one of SEQ ID NOs: 32-52, 174-177, 207, or 208; and/or the immunoglobulin light chain variable region polypeptide comprises a complementarity determining region 1 (LCDR1) comprising any one of SEQ ID NOs: 53-67 or 178-182, a complementarity determining region 2 (LCDR2) comprising any one of SEQ ID NOs: 68-79, and a complementarity determining region 3 (LCDR3) comprising any one of SEQ ID NOs: 80-91. Also provided are nucleic acids encoding the PD-L1 binding agents, or the individual heavy and light chains thereof; vectors and cells comprising the nucleic acids; and compositions comprising the binding agents or nucleic acids.

Also provided is a method of preparing a binding agent as described herein, which method comprises expressing in a cell one or more nucleic acids encoding the heavy and light chain variable region polypeptides of the binding agent.

Further provided is a method of delivering a payload to a cell expressing PD-L1 comprising administering to the cell, or mammal comprising the cell, a PD-L1 binding agent provided herein conjugated to the payload.

Further provided is a method for enhancing or inhibiting an immune response in a mammal, and a method for treating a disease, disorder, or condition in a mammal that is responsive to PD-L1 inhibition, which methods comprise administering a binding agent as described herein, or conjugate thereof, to the mammal.

Additional aspects and embodiments of the invention are as provided in the following detailed description.

### BRIEF DESCRIPTION OF THE SEVERAL VIEWS OF THE DRAWING(S)

FIG. 1 depicts experimental data illustrating the affinity for binding agents of an embodiment of the invention for human PD-L1 on JIMT-1 cell surface.

FIG. 2 depicts experimental data illustrating the affinity for binding agents of an embodiment of the invention for human PD-L1 using JIMT-1 cell.

FIG. 3 depicts experimental data illustrating the level of cellular internalization of binding agents of an embodiment of the invention.

FIG. 4 depicts experimental data illustrating the affinity for binding agents of an embodiment of the invention for human PD-L1 on JIMT-1 cell surface.

### DETAILED DESCRIPTION OF THE INVENTION

The invention provides a PD-L1 binding agent comprising an immunoglobulin heavy chain variable region polypeptide and an immunoglobulin light chain variable region polypeptide.

The PD-L1 binding agent specifically binds PD-L1. The binding specificity of the agent allows for targeting PD-L1 expressing cells, for instance, to deliver therapeutic payloads to such cells. In some embodiments, the PD-L1 binding agent completely or partially blocks (inhibits or prevents) binding of PD-L1 to its receptor, PD-1, such that the antibody can be used to inhibit PD-L1/PD-1 signaling (e.g., for therapeutic purposes).

Furthermore, in some embodiments, the PD-L1 binding agents provided herein do not cause any significant cellular internalization of PD-L1 or the PD-L1/PD-L1 binding agent complex upon binding to PD-L1 on the cell surface. Cellular internalization of PD-L1 and bound PD-L1 binding agent can be determined by any suitable method, such as assaying for persistence on the cell surface and/or detection of internalized antibodies. In some embodiments, the PD-L1 binding agent does not significantly internalize, such that at least about 75% or about 80% (e.g., at least about 85%, at least about 90%, or at least about 95%) of the PD-L1 binding agent that binds PD-L1 on the cell surface remains on the cell surface (e.g., using a surface persistence assay, about 75% or more, about 80% or more, about 85% or more, about 90% or more, or about 95% or more of the PD-L1 binding agent molecules bound to PD-L1 on the cell surface at the beginning of the assay remain bound at the end of the assay). Persistence can be tested over a suitable length of time long enough to allow internalization to occur. In some embodiments, the PD-L1 binding agent remains on the cell surface as described above for about 12 hours or more, or about 24 hrs or more, such as about 2 days or more, or even 3 days or more.

In some embodiments, the PD-L1 binding agent binds to human PD-L1, for example, a protein comprising SEQ ID NO: 165 (MRIFAVFIFMTYWHLLNAFTVTVPKDLYVVEYGSNMTIECKFPVEKQLDLAALIVY WEMEDKNIIQFVHGEEDLKVQHSSYRQRARLLKDQLSLGNAALQITDVKLQDAGVY RCMISYGGADYKRITVKVNAPYNKINQRILVVDPVTSEHELTCQAEGYPKAEVIWTS SDHQVLSGKTTTTNSKREEKLFNVTSTLRINTTTNEIFYCTFRRLDPEENHTAELVIPE LPLAHPPNERTHLVILGAILLCLGVALTFIFRLRKGRMMDVKKCGIQDTNSKKQSDTH LEET). However, binding agents that bind to any PD-L1 homolog or paralog also are encompassed. In some embodiments, the PD-L1 protein comprises at least about 70%, about 75%, about 80%, about 85%, about 90%, about 91%, about 92%, about 93%, about 94%, about 95%, about 96%, about 97%, about 98%, about 99%, or more sequence identity to SEQ ID NO: 165). In some embodiments, the binding agent binds human PD-L1 and cynomolgus PD-L1; or human, cynomolgus and mouse PD-L1.

Nucleic acid or amino acid sequence “identity,” as referenced herein, can be determined by comparing a nucleic acid or amino acid sequence of interest to a reference nucleic acid or amino acid sequence. The percent identity is the number of nucleotides or amino acid residues that are the same (i.e., that are identical) as between the optimally aligned sequence of interest and the reference sequence divided by the length of the longest sequence (i.e., the length of either the sequence of interest or the reference sequence, whichever is longer). Alignment of sequences and calculation of percent identity can be performed using available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3×, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)). Percent (%) identity of sequences can be also calculated, for example, as 100×[(identical positions)/min(TG_{A}, TG_{B})], where TG_{A} and TG_{B} are the sum of the number of residues and internal gap positions in peptide sequences A and B in the alignment that minimizes TG_{A} and TG_{B}. See, e.g., Russell et al., J. Mol Biol., 244: 332-350 (1994).

The binding agent comprises Ig heavy and light chain variable region polypeptides that together form the antigen binding site. Each of the heavy and light chain variable regions are polypeptides comprising three complementarity determining regions (CDR1, CDR2, and CDR3) connected by framework regions. The binding agent can be any of a variety of types of binding agents known in the art that comprise Ig heavy and light chains. For instance, the binding agent can be an antibody, an antigen-binding antibody “fragment,” or a T-cell receptor.

In some embodiments, the binding agent is a whole (or complete) antibody, which comprises an antigen binding domain comprising the Ig heavy and light variable domains as well as a fragment crystallizable (Fc) domain. An exemplary antibody structure is a tetramer composed of two pairs of polypeptide chains, each pair having one “light” (a smaller chain, such as about 25 kDa) and one “heavy” chain (a larger chain, such as about 50-70 kDa), typically connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region, typically about 100 to 110 or more amino acids (but not limited thereto), referred to as the paratope, primarily responsible for antigen recognition, i.e., the antigen binding domain. Light chains are classified as either kappa or lambda. Heavy chains are classified as gamma, mu, alpha, delta, or epsilon, which in turn define the immunoglobulin classes, IgG, IgM, IgA, IgD and IgE, respectively. The classes can be further divided into subclasses. For instance, there are four IgG subclasses (IgG1, IgG2, IgG3, and IgG4) in humans, named in order of their abundance in serum (i.e., IgG1 is the most abundant).

In some embodiments, the binding agent is an antigen-binding antibody “fragment,” which is a construct that comprises at least an antigen-binding region of an antibody, alone or with other components that together constitute the antigen-binding construct. Many different types of antibody “fragments” are known in the art, including, for instance, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a single chain Fv (scFv), which is a monovalent molecule consisting of the two domains of the Fv fragment (i.e., V_{L} and V_{H}) joined by a synthetic linker which enables the two domains to be synthesized as a single polypeptide chain.

The antibody or antibody fragments can be part of a larger construct, for example, a conjugate or fusion construct of the antibody fragment to additional regions. For instance, in some embodiments, the antibody fragment can be fused to an Fc region as described herein. In other embodiments, the antibody fragment (e.g., a Fab or scFv) can be part of a chimeric antigen receptor or chimeric T-cell receptor, for instance, by fusing to a transmembrane domain (optionally with an intervening linker or “stalk” (e.g., hinge region)) and optional intercellular signaling domain. For instance, the antibody fragment can be fused to the gamma and/or delta chains of a t-cell receptor, so as to provide a T-cell receptor like construct that binds PD-L1. In yet another embodiment, the antibody fragment is part of a bispecific T-cell engager (BiTEs) comprising a CD1 or CD3 binding domain and linker.

The antibody or antigen-binding antibody fragment can be monospecific for PD-L1, or can be bispecific or multi-specific. For instance, in bivalent or multivalent antibodies or antibody fragments, the binding domains can be different targeting different epitopes of the same antigen or targeting different antigens. Methods of constructing multivalent binding constructs are known in the art. Bispecific and multispecific antibodies are known in the art. Furthermore, a diabody, triabody, or tetrabody can be provided, which is a dimer, trimer, or tetramer of polypeptide chains each comprising a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a multimeric molecule having two, three, or four functional antigen binding sites. Also, bis-scFv fragments, which are small scFv fragments with two different variable domains can be generated to produce bispecific bis-scFv fragments capable of binding two different epitopes. Fab dimers (Fab2) and Fab trimers (Fab3) can be produced using genetic engineering methods to create multispecific constructs based on Fab fragments.

The PD-L1-binding agent also can be an antibody conjugate. In this respect, the PD-L1-binding agent can be a conjugate of (1) an antibody, an alternative scaffold, or fragments thereof, and (2) a protein or non-protein moiety. For example, the PD-L1 binding agent can be conjugated to a peptide, a fluorescent molecule, chemotherapeutic or other cytotoxic payload, immune-activating or immune-suppressive agent.

The PD-L1-binding agent can be, or can be obtained from, a human antibody, a non-human antibody, a humanized antibody, or a chimeric antibody, or corresponding antibody fragments. A “chimeric” antibody is an antibody or fragment thereof typically comprising human constant regions and non-human variable regions. A “humanized” antibody is a monoclonal antibody typically comprising a human antibody scaffold but with non-human origin amino acids or sequences in at least one CDR (e.g., 1, 2, 3, 4, 5, or all six CDRs).

Methods for generating such antibodies are known in the art and are described in, for example, Köhler and Milstein, Eur. J. Immunol., 5: 511-519 (1976); Harlow and Lane (eds.), Antibodies: A Laboratory Manual, CSH Press (1988); and Janeway et al. (eds.), Immunobiology, 9th Ed., Garland Publishing, New York, N.Y. (2017). In certain embodiments, a human or chimeric antibody or antibody fragment can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the Medarex HUMAB-MOUSE™, the Kirin TC MOUSE™, and the Kyowa Kirin KM-MOUSE™ (see, e.g., Lonberg, Nat. Biotechnol., 23(9): 1117-25 (2005), and Lonberg, Handb. Exp. Pharmacol., 181: 69-97 (2008)). A humanized antibody can be generated using any suitable method known in the art (see, e.g., An, Z. (ed.), Therapeutic Monoclonal Antibodies: From Bench to Clinic, John Wiley & Sons, Inc., Hoboken, N.J. (2009)), including, e.g., grafting of non-human CDRs onto a human antibody scaffold (see, e.g., Kashmiri et al., Methods, 36(1): 25-34 (2005); and Hou et al., J. Biochem., 144(1): 115-120 (2008) and use of phage display (see, e.g., Fellouse, et al., Journal of Molecular Biology, 373(4): 924-940 (2007) and Glanville, et al., PNAS, 106(48): 20216-20221 (2009)).

In an embodiment, the PD-L1 binding agent comprises an immunoglobulin heavy chain variable region of any one of SEQ ID NOs: 123-143, 184-193, or 209-213, a sequence that is at least about 90%, at least about 91%, at least about 92%, at least about 93%, at least about 94%, at least about 95%, at least about 96%, at least about 97%, at least about 98%, or at least about 99% identical to any one of SEQ ID NOs: 123-143, 184-193, or 209-213, or at least the CDRs thereof; and/or an immunoglobulin light chain variable region of any one of SEQ ID NOs: 144-164 or 194-202, a sequence that is at least about 90%, at least about 91%, at least about 92%, at least about 93%, at least about 94%, at least about 95%, at least about 96%, at least about 97%, at least about 98%, or at least about 99% identical to any one of SEQ ID NOs: 144-164 or 194-202, or at least the CDRs thereof.

By way of further illustration, the PD-L1 binding agent can comprise:

(1) an immunoglobulin heavy chain variable region of SEQ ID NO: 123, or at least the CDRs thereof, and/or an immunoglobulin light chain variable region of SEQ ID NO: 144, or at least the CDRs thereof;

(2) an immunoglobulin heavy chain variable region of SEQ ID NO: 124, or at least the CDRs thereof, and/or an immunoglobulin light chain variable region of SEQ ID NO: 145, or at least the CDRs thereof;

(3) an immunoglobulin heavy chain variable region of SEQ ID NO: 125, or at least the CDRs thereof, and/or an immunoglobulin light chain variable region of SEQ ID NO: 146, or at least the CDRs thereof;

(4) an immunoglobulin heavy chain variable region of SEQ ID NO: 126, or at least the CDRs thereof, and/or an immunoglobulin light chain variable region of SEQ ID NO: 147, or at least the CDRs thereof;

(5) an immunoglobulin heavy chain variable region of SEQ ID NO: 127, or at least the CDRs thereof, and/or an immunoglobulin light chain variable region of SEQ ID NO: 148, or at least the CDRs thereof;

(6) an immunoglobulin heavy chain variable region of SEQ ID NO: 128, or at least the CDRs thereof, and/or an immunoglobulin light chain variable region of SEQ ID NO: 149, or at least the CDRs thereof;

(7) an immunoglobulin heavy chain variable region of SEQ ID NO: 129, or at least the CDRs thereof, and/or an immunoglobulin light chain variable region of SEQ ID NO: 150, or at least the CDRs thereof;

(8) an immunoglobulin heavy chain variable region of SEQ ID NO: 130, or at least the CDRs thereof, and/or an immunoglobulin light chain variable region of SEQ ID NO: 151, or at least the CDRs thereof;

(9) an immunoglobulin heavy chain variable region of SEQ ID NO: 131, or at least the CDRs thereof, and/or an immunoglobulin light chain variable region of SEQ ID NO: 152, or at least the CDRs thereof;

(10) an immunoglobulin heavy chain variable region of SEQ ID NO: 132, or at least the CDRs thereof, and/or an immunoglobulin light chain variable region of SEQ ID NO: 153, or at least the CDRs thereof;

(11) an immunoglobulin heavy chain variable region of SEQ ID NO: 133, or at least the CDRs thereof, and/or an immunoglobulin light chain variable region of SEQ ID NO: 154, or at least the CDRs thereof;

(12) an immunoglobulin heavy chain variable region of SEQ ID NO: 134, or at least the CDRs thereof, and/or an immunoglobulin light chain variable region of SEQ ID NO: 155, or at least the CDRs thereof;

(13) an immunoglobulin heavy chain variable region of SEQ ID NO: 135, or at least the CDRs thereof, and/or an immunoglobulin light chain variable region of SEQ ID NO: 156, or at least the CDRs thereof;

(14) an immunoglobulin heavy chain variable region of SEQ ID NO: 136, or at least the CDRs thereof, and/or an immunoglobulin light chain variable region of SEQ ID NO: 157, or at least the CDRs thereof;

(15) an immunoglobulin heavy chain variable region of SEQ ID NO: 137, or at least the CDRs thereof, and/or an immunoglobulin light chain variable region of SEQ ID NO: 158, or at least the CDRs thereof;

(16) an immunoglobulin heavy chain variable region of SEQ ID NO: 138, or at least the CDRs thereof, and/or an immunoglobulin light chain variable region of SEQ ID NO: 159, or at least the CDRs thereof;

(17) an immunoglobulin heavy chain variable region of SEQ ID NO: 139, or at least the CDRs thereof, and/or an immunoglobulin light chain variable region of SEQ ID NO: 160, or at least the CDRs thereof;

(18) an immunoglobulin heavy chain variable region of SEQ ID NO: 140, or at least the CDRs thereof, and/or an immunoglobulin light chain variable region of SEQ ID NO: 161, or at least the CDRs thereof;

(19) an immunoglobulin heavy chain variable region of SEQ ID NO: 141, or at least the CDRs thereof, and/or an immunoglobulin light chain variable region of SEQ ID NO: 162, or at least the CDRs thereof; and/or

(20) an immunoglobulin heavy chain variable region of SEQ ID NO: 142, or at least the CDRs thereof, and/or an immunoglobulin light chain variable region of SEQ ID NO: 163, or at least the CDRs thereof;

(21) an immunoglobulin heavy chain variable region of SEQ ID NO: 143, or at least the CDRs thereof, and/or an immunoglobulin light chain variable region of SEQ ID NO: 164, or at least the CDRs thereof;

(22) an immunoglobulin heavy chain variable region of SEQ ID NO: 184, or at least the CDRs thereof, and/or an immunoglobulin light chain variable region of SEQ ID NO: 194, or at least the CDRs thereof;

(23) an immunoglobulin heavy chain variable region of SEQ ID NO: 185 (wherein X can be any amino acid), or at least the CDRs thereof, and/or an immunoglobulin light chain variable region of SEQ ID NO: 194, or at least the CDRs thereof;

(24) an immunoglobulin heavy chain variable region of SEQ ID NO: 186 (wherein X can be any amino acid), or at least the CDRs thereof, and/or an immunoglobulin light chain variable region of SEQ ID NO: 194, or at least the CDRs thereof;

(25) an immunoglobulin heavy chain variable region of SEQ ID NO: 187, or at least the CDRs thereof, and/or an immunoglobulin light chain variable region of SEQ ID NO: 194, or at least the CDRs thereof;

(26) an immunoglobulin heavy chain variable region of SEQ ID NO: 188, or at least the CDRs thereof, and/or an immunoglobulin light chain variable region of SEQ ID NO: 194, or at least the CDRs thereof;

(27) an immunoglobulin heavy chain variable region of SEQ ID NO: 189, or at least the CDRs thereof, and/or an immunoglobulin light chain variable region of SEQ ID NO: 194, or at least the CDRs thereof;

(28) an immunoglobulin heavy chain variable region of SEQ ID NO: 189, or at least the CDRs thereof, and/or an immunoglobulin light chain variable region of SEQ ID NO: 194, or at least the CDRs thereof;

(29) an immunoglobulin heavy chain variable region of SEQ ID NO: 190, or at least the CDRs thereof, and/or an immunoglobulin light chain variable region of SEQ ID NO: 194, or at least the CDRs thereof;

(30) an immunoglobulin heavy chain variable region of SEQ ID NO: 191, or at least the CDRs thereof, and/or an immunoglobulin light chain variable region of SEQ ID NO: 194, or at least the CDRs thereof;

(31) an immunoglobulin heavy chain variable region of SEQ ID NO: 192, or at least the CDRs thereof, and/or an immunoglobulin light chain variable region of SEQ ID NO: 194, or at least the CDRs thereof;

(32) an immunoglobulin heavy chain variable region of SEQ ID NO: 193, or at least the CDRs thereof, and/or an immunoglobulin light chain variable region of SEQ ID NO: 194, or at least the CDRs thereof;

(33) an immunoglobulin heavy chain variable region of SEQ ID NO: 130, or at least the CDRs thereof, and/or an immunoglobulin light chain variable region of SEQ ID NO: 194, or at least the CDRs thereof;

(34) an immunoglobulin heavy chain variable region of SEQ ID NO: 188, or at least the CDRs thereof, and/or an immunoglobulin light chain variable region of SEQ ID NO: 195, or at least the CDRs thereof;

(35) an immunoglobulin heavy chain variable region of SEQ ID NO: 188, or at least the CDRs thereof, and/or an immunoglobulin light chain variable region of SEQ ID NO: 196, or at least the CDRs thereof;

(36) an immunoglobulin heavy chain variable region of SEQ ID NO: 188, or at least the CDRs thereof, and/or an immunoglobulin light chain variable region of SEQ ID NO: 197, or at least the CDRs thereof;

(37) an immunoglobulin heavy chain variable region of SEQ ID NO: 188, or at least the CDRs thereof, and/or an immunoglobulin light chain variable region of SEQ ID NO: 198, or at least the CDRs thereof;

(38) an immunoglobulin heavy chain variable region of SEQ ID NO: 188, or at least the CDRs thereof, and/or an immunoglobulin light chain variable region of SEQ ID NO: 199, or at least the CDRs thereof;

(39) an immunoglobulin heavy chain variable region of SEQ ID NO: 188, or at least the CDRs thereof, and/or an immunoglobulin light chain variable region of SEQ ID NO: 200, or at least the CDRs thereof;

(40) an immunoglobulin heavy chain variable region of SEQ ID NO: 188, or at least the CDRs thereof, and/or an immunoglobulin light chain variable region of SEQ ID NO: 157, or at least the CDRs thereof;

(41) an immunoglobulin heavy chain variable region of SEQ ID NO: 188, or at least the CDRs thereof, and/or an immunoglobulin light chain variable region of SEQ ID NO: 201, or at least the CDRs thereof;

(42) an immunoglobulin heavy chain variable region of SEQ ID NO: 188, or at least the CDRs thereof, and/or an immunoglobulin light chain variable region of SEQ ID NO: 202, or at least the CDRs thereof;

(43) an immunoglobulin heavy chain variable region of SEQ ID NO: 188, or at least the CDRs thereof, and/or an immunoglobulin light chain variable region of SEQ ID NO: 202, or at least the CDRs thereof;

(44) an immunoglobulin heavy chain variable region of SEQ ID NO: 188, or at least the CDRs thereof, and/or an immunoglobulin light chain variable region of SEQ ID NO: 194, or at least the CDRs thereof;

(45) an immunoglobulin heavy chain variable region of SEQ ID NO: 209, or at least the CDRs thereof, and/or an immunoglobulin light chain variable region of SEQ ID NO: 144, or at least the CDRs thereof;

(46) an immunoglobulin heavy chain variable region of SEQ ID NO: 210, or at least the CDRs thereof, and/or an immunoglobulin light chain variable region of SEQ ID NO: 148, or at least the CDRs thereof;

(47) an immunoglobulin heavy chain variable region of SEQ ID NO: 211, or at least the CDRs thereof, and/or an immunoglobulin light chain variable region of SEQ ID NO: 153, or at least the CDRs thereof;

(48) an immunoglobulin heavy chain variable region of SEQ ID NO: 212, or at least the CDRs thereof, and/or an immunoglobulin light chain variable region of SEQ ID NO: 150, or at least the CDRs thereof;

(49) an immunoglobulin heavy chain variable region of SEQ ID NO: 213, or at least the CDRs thereof, and/or an immunoglobulin light chain variable region of SEQ ID NO: 152, or at least the CDRs thereof; and/or

(50) an immunoglobulin heavy chain variable region of Table 4 and/or an immunoglobulin light chain variable region of Table 4, or at least the CDRs thereof.

The CDRs of a given heavy or light chain Ig sequence can be determined in accordance with any of the various known Ig numbering schemes (e.g., Kabat, Chothia, Martin (Enhanced Chothia), IGMT, AbM). In certain embodiments, the PD-L1 binding agent comprises one or more of the following CDRs:

a HCDR1 comprising or consisting of any one of SEQ ID NOs: 1-14 or 167 or a sequence that is at least about 90%, at least about 91%, at least about 92%, at least about 93%, at least about 94%, at least about 95%, at least about 96%, at least about 97%, at least about 98%, or at least about 99% identical to any one of SEQ ID NOs: 1-14 or 167;

a HCDR2 comprising or consisting of any one of SEQ ID NOs: 15-31, 168-173, or 203-206, or a sequence that is at least about 90%, at least about 91%, at least about 92%, at least about 93%, at least about 94%, at least about 95%, at least about 96%, at least about 97%, at least about 98%, or at least about 99% identical to any one of SEQ ID NOs: 15-31, 168-173, or 203-206; and

a HCDR3 comprising or consisting of any one of SEQ ID NOs: 32-52, 174-177, 207, or 208, or a sequence that is at least about 90%, at least about 91%, at least about 92%, at least about 93%, at least about 94%, at least about 95%, at least about 96%, at least about 97%, at least about 98%, or at least about 99% identical to any one of SEQ ID NOs: 32-52, 174-177, 207, or 208; and/or the immunoglobulin light chain polypeptide comprises

a LCDR1 comprising or consisting of any one of SEQ ID NOs: 53-67 or 178-182 or a sequence that is at least about 90%, at least about 91%, at least about 92%, at least about 93%, at least about 94%, at least about 95%, at least about 96%, at least about 97%, at least about 98%, or at least about 99% identical to any one of SEQ ID NOs: 53-67 or 178-182;

a LCDR2 comprising or consisting of any one of SEQ ID NOs: 68-79 or a sequence that is at least about 90%, at least about 91%, at least about 92%, at least about 93%, at least about 94%, at least about 95%, at least about 96%, at least about 97%, at least about 98%, or at least about 99% identical to any one of SEQ ID NOs: 68-79; and

a LCDR3 comprising or consisting of any one of SEQ ID NOs: 80-91 or a sequence that is at least about 90%, at least about 91%, at least about 92%, at least about 93%, at least about 94%, at least about 95%, at least about 96%, at least about 97%, at least about 98%, or at least about 99% identical to any one of SEQ ID NOs: 80-91.

In particular embodiments, the binding agent comprises an immunoglobulin heavy chain polypeptide and an immunoglobulin light chain polypeptide, wherein:

(1) the immunoglobulin heavy chain polypeptide comprises a HCDR1 comprising or consisting of SEQ ID NO: 1, a HCDR2 comprising or consisting of SEQ ID NO: 15, and a HCDR3 comprising or consisting of SEQ ID NO: 32; and/or the immunoglobulin light chain polypeptide comprises a LCDR1 comprising or consisting of SEQ ID NO: 53, a LCDR2 comprising or consisting of SEQ ID NO: 68, and a LCDR3 comprising or consisting of SEQ ID NO: 80;

(2) the immunoglobulin heavy chain polypeptide comprises a HCDR1 comprising or consisting of SEQ ID NO: 2, a HCDR2 comprising or consisting of SEQ ID NO: 16, and a HCDR3 comprising or consisting of SEQ ID NO: 33; and/or the immunoglobulin light chain polypeptide comprises a LCDR1 comprising or consisting of SEQ ID NO: 54, a LCDR2 comprising or consisting of SEQ ID NO: 69, and a LCDR3 comprising or consisting of SEQ ID NO: 81;

(3) the immunoglobulin heavy chain polypeptide comprises a HCDR1 comprising or consisting of SEQ ID NO: 3, a HCDR2 comprising or consisting of SEQ ID NO: 17, and a HCDR3 comprising or consisting of SEQ ID NO: 34; and/or the immunoglobulin light chain polypeptide comprises a LCDR1 comprising or consisting of SEQ ID NO: 53, a LCDR2 comprising or consisting of SEQ ID NO: 68, and a LCDR3 comprising or consisting of SEQ ID NO: 80;

(4) the immunoglobulin heavy chain polypeptide comprises a HCDR1 comprising or consisting of SEQ ID NO: 4, a HCDR2 comprising or consisting of SEQ ID NO: 18, and a HCDR3 comprising or consisting of SEQ ID NO: 35; and/or the immunoglobulin light chain polypeptide comprises a LCDR1 comprising or consisting of SEQ ID NO: 55, a LCDR2 comprising or consisting of SEQ ID NO: 70, and a LCDR3 comprising or consisting of SEQ ID NO: 82;

(5) the immunoglobulin heavy chain polypeptide comprises a HCDR1 comprising or consisting of SEQ ID NO: 5, a HCDR2 comprising or consisting of SEQ ID NO: 19, and a HCDR3 comprising or consisting of SEQ ID NO: 36; and/or the immunoglobulin light chain polypeptide comprises a LCDR1 comprising or consisting of SEQ ID NO: 53, a LCDR2 comprising or consisting of SEQ ID NO: 71, and a LCDR3 comprising or consisting of SEQ ID NO: 80;

(6) the immunoglobulin heavy chain polypeptide comprises a HCDR1 comprising or consisting of SEQ ID NO: 6, a HCDR2 comprising or consisting of SEQ ID NO: 20, and a HCDR3 comprising or consisting of SEQ ID NO: 37; and/or the immunoglobulin light chain polypeptide comprises a LCDR1 comprising or consisting of SEQ ID NO: 56, a LCDR2 comprising or consisting of SEQ ID NO: 72, and a LCDR3 comprising or consisting of SEQ ID NO: 83;

(7) the immunoglobulin heavy chain polypeptide comprises a HCDR1 comprising or consisting of SEQ ID NO: 7, a HCDR2 comprising or consisting of SEQ ID NO: 20, and a HCDR3 comprising or consisting of SEQ ID NO: 38; and/or the immunoglobulin light chain polypeptide comprises a LCDR1 comprising or consisting of SEQ ID NO: 57, a LCDR2 comprising or consisting of SEQ ID NO: 73, and a LCDR3 comprising or consisting of SEQ ID NO: 84;

(8) the immunoglobulin heavy chain polypeptide comprises a HCDR1 comprising or consisting of SEQ ID NO: 5, a HCDR2 comprising or consisting of SEQ ID NO: 21, and a HCDR3 comprising or consisting of SEQ ID NO: 39; and/or the immunoglobulin light chain polypeptide comprises a LCDR1 comprising or consisting of SEQ ID NO: 58, a LCDR2 comprising or consisting of SEQ ID NO: 68, and a LCDR3 comprising or consisting of SEQ ID NO: 80;

(9) the immunoglobulin heavy chain polypeptide comprises a HCDR1 comprising or consisting of SEQ ID NO: 7, a HCDR2 comprising or consisting of SEQ ID NO: 22, and a HCDR3 comprising or consisting of SEQ ID NO: 40; and/or the immunoglobulin light chain polypeptide comprises a LCDR1 comprising or consisting of SEQ ID NO: 59, a LCDR2 comprising or consisting of SEQ ID NO: 68, and a LCDR3 comprising or consisting of SEQ ID NO: 82;

(10) the immunoglobulin heavy chain polypeptide comprises a HCDR1 comprising or consisting of SEQ ID NO: 2, a HCDR2 comprising or consisting of SEQ ID NO: 23, and a HCDR3 comprising or consisting of SEQ ID NO: 41; and/or the immunoglobulin light chain polypeptide comprises a LCDR1 comprising or consisting of SEQ ID NO: 53, a LCDR2 comprising or consisting of SEQ ID NO: 74, and a LCDR3 comprising or consisting of SEQ ID NO: 85;

(11) the immunoglobulin heavy chain polypeptide comprises a HCDR1 comprising or consisting of SEQ ID NO: 2, a HCDR2 comprising or consisting of SEQ ID NO: 20, and a HCDR3 comprising or consisting of SEQ ID NO: 42; and/or the immunoglobulin light chain polypeptide comprises a LCDR1 comprising or consisting of SEQ ID NO: 60, a LCDR2 comprising or consisting of SEQ ID NO: 75, and a LCDR3 comprising or consisting of SEQ ID NO: 82;

(12) the immunoglobulin heavy chain polypeptide comprises a HCDR1 comprising or consisting of SEQ ID NO: 2, a HCDR2 comprising or consisting of SEQ ID NO: 15, and a HCDR3 comprising or consisting of SEQ ID NO: 43; and/or the immunoglobulin light chain polypeptide comprises a LCDR1 comprising or consisting of SEQ ID NO: 53, a LCDR2 comprising or consisting of SEQ ID NO: 76, and a LCDR3 comprising or consisting of SEQ ID NO: 80;

(13) the immunoglobulin heavy chain polypeptide comprises a HCDR1 comprising or consisting of SEQ ID NO: 8, a HCDR2 comprising or consisting of SEQ ID NO: 16, and a HCDR3 comprising or consisting of SEQ ID NO: 44; and/or the immunoglobulin light chain polypeptide comprises a LCDR1 comprising or consisting of SEQ ID NO: 61, a LCDR2 comprising or consisting of SEQ ID NO: 68, and a LCDR3 comprising or consisting of SEQ ID NO: 86;

(14) the immunoglobulin heavy chain polypeptide comprises a HCDR1 comprising or consisting of SEQ ID NO: 9, a HCDR2 comprising or consisting of SEQ ID NO: 24, and a HCDR3 comprising or consisting of SEQ ID NO: 45; and/or the immunoglobulin light chain polypeptide comprises a LCDR1 comprising or consisting of SEQ ID NO: 58, a LCDR2 comprising or consisting of SEQ ID NO: 68, and a LCDR3 comprising or consisting of SEQ ID NO: 82;

(15) the immunoglobulin heavy chain polypeptide comprises a HCDR1 comprising or consisting of SEQ ID NO: 10, a HCDR2 comprising or consisting of SEQ ID NO: 25, and a HCDR3 comprising or consisting of SEQ ID NO: 46; and/or the immunoglobulin light chain polypeptide comprises a LCDR1 comprising or consisting of SEQ ID NO: 62, a LCDR2 comprising or consisting of SEQ ID NO: 77, and a LCDR3 comprising or consisting of SEQ ID NO: 87;

(16) the immunoglobulin heavy chain polypeptide comprises a HCDR1 comprising or consisting of SEQ ID NO: 11, a HCDR2 comprising or consisting of SEQ ID NO: 26, and a HCDR3 comprising or consisting of SEQ ID NO: 47; and/or the immunoglobulin light chain polypeptide comprises a LCDR1 comprising or consisting of SEQ ID NO: 63, a LCDR2 comprising or consisting of SEQ ID NO: 72, and a LCDR3 comprising or consisting of SEQ ID NO: 88;

(17) the immunoglobulin heavy chain polypeptide comprises a HCDR1 comprising or consisting of SEQ ID NO: 3, a HCDR2 comprising or consisting of SEQ ID NO: 27, and a HCDR3 comprising or consisting of SEQ ID NO: 48; and/or the immunoglobulin light chain polypeptide comprises a LCDR1 comprising or consisting of SEQ ID NO: 64, a LCDR2 comprising or consisting of SEQ ID NO: 68, and a LCDR3 comprising or consisting of SEQ ID NO: 80;

(18) the immunoglobulin heavy chain polypeptide comprises a HCDR1 comprising or consisting of SEQ ID NO: 3, a HCDR2 comprising or consisting of SEQ ID NO: 28, and a HCDR3 comprising or consisting of SEQ ID NO: 49; and/or the immunoglobulin light chain polypeptide comprises a LCDR1 comprising or consisting of SEQ ID NO: 53, a LCDR2 comprising or consisting of SEQ ID NO: 78, and a LCDR3 comprising or consisting of SEQ ID NO: 89;

(19) the immunoglobulin heavy chain polypeptide comprises a HCDR1 comprising or consisting of SEQ ID NO: 12, a HCDR2 comprising or consisting of SEQ ID NO: 29, and a HCDR3 comprising or consisting of SEQ ID NO: 50; and/or the immunoglobulin light chain polypeptide comprises a LCDR1 comprising or consisting of SEQ ID NO: 65, a LCDR2 comprising or consisting of SEQ ID NO: 79, and a LCDR3 comprising or consisting of SEQ ID NO: 90;

(20) the immunoglobulin heavy chain polypeptide comprises a HCDR1 comprising or consisting of SEQ ID NO: 13, a HCDR2 comprising or consisting of SEQ ID NO: 30, and a HCDR3 comprising or consisting of SEQ ID NO: 51; and/or the immunoglobulin light chain polypeptide comprises a LCDR1 comprising or consisting of SEQ ID NO: 66, a LCDR2 comprising or consisting of SEQ ID NO: 72, and a LCDR3 comprising or consisting of SEQ ID NO: 91;

(21) the immunoglobulin heavy chain polypeptide comprises a HCDR1 comprising or consisting of SEQ ID NO: 14, a HCDR2 comprising or consisting of SEQ ID NO: 31, and a HCDR3 comprising or consisting of SEQ ID NO: 52; and/or the immunoglobulin light chain polypeptide comprises a LCDR1 comprising or consisting of SEQ ID NO: 67, a LCDR2 comprising or consisting of SEQ ID NO: 72, and a LCDR3 comprising or consisting of SEQ ID NO: 82;

(22) the immunoglobulin heavy chain polypeptide comprises a HCDR1 comprising or consisting of SEQ ID NO: 167, a HCDR2 comprising or consisting of SEQ ID NO: 168, and a HCDR3 comprising or consisting of SEQ ID NO: 174; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(23) the immunoglobulin heavy chain polypeptide comprises a HCDR1 comprising or consisting of SEQ ID NO: 13, a HCDR2 comprising or consisting of SEQ ID NO: 169, and a HCDR3 comprising or consisting of SEQ ID NO: 175 (ERFLGGXMDV, wherein X can be any amino acid); and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(24) the immunoglobulin heavy chain polypeptide comprises a HCDR1 comprising or consisting of SEQ ID NO: 167, a HCDR2 comprising or consisting of SEQ ID NO: 170, and a HCDR3 comprising or consisting of SEQ ID NO: 176 (EAVAGPXMDV, wherein X can be any amino acid); and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(25) the immunoglobulin heavy chain polypeptide comprises a HCDR1 comprising or consisting of SEQ ID NO: 167, a HCDR2 comprising or consisting of SEQ ID NO: 169, and a HCDR3 comprising or consisting of SEQ ID NO: 177; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(26) the immunoglobulin heavy chain polypeptide comprises a HCDR1 comprising or consisting of SEQ ID NO: 5, a HCDR2 comprising or consisting of SEQ ID NO: 171, and a HCDR3 comprising or consisting of SEQ ID NO: 39; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(27) the immunoglobulin heavy chain polypeptide comprises a HCDR1 comprising or consisting of SEQ ID NO: 167, a HCDR2 comprising or consisting of SEQ ID NO: 168, and a HCDR3 comprising or consisting of SEQ ID NO: 174; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(28) the immunoglobulin heavy chain polypeptide comprises a HCDR1 comprising or consisting of SEQ ID NO: 167, a HCDR2 comprising or consisting of SEQ ID NO: 168, and a HCDR3 comprising or consisting of SEQ ID NO: 174; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(29) the immunoglobulin heavy chain polypeptide comprises a HCDR1 comprising or consisting of SEQ ID NO: 167, a HCDR2 comprising or consisting of SEQ ID NO: 172, and a HCDR3 comprising or consisting of SEQ ID NO: 174; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(30) the immunoglobulin heavy chain polypeptide comprises a HCDR1 comprising or consisting of SEQ ID NO: 167, a HCDR2 comprising or consisting of SEQ ID NO: 173, and a HCDR3 comprising or consisting of SEQ ID NO: 174; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(31) the immunoglobulin heavy chain polypeptide comprises a HCDR1 comprising or consisting of SEQ ID NO: 167, a HCDR2 comprising or consisting of SEQ ID NO: 172, and a HCDR3 comprising or consisting of SEQ ID NO: 174; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(32) the immunoglobulin heavy chain polypeptide comprises a HCDR1 comprising or consisting of SEQ ID NO: 167, a HCDR2 comprising or consisting of SEQ ID NO: 170, and a HCDR3 comprising or consisting of SEQ ID NO: 174; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(33) the immunoglobulin heavy chain polypeptide comprises a HCDR1 comprising or consisting of SEQ ID NO: 5, a HCDR2 comprising or consisting of SEQ ID NO: 21, and a HCDR3 comprising or consisting of SEQ ID NO: 39; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(34) the immunoglobulin heavy chain polypeptide comprises a HCDR1 comprising or consisting of SEQ ID NO: 5, a HCDR2 comprising or consisting of SEQ ID NO: 171, and a HCDR3 comprising or consisting of SEQ ID NO: 39; and/or the immunoglobulin light chain polypeptide comprises a LCDR1 comprising or consisting of SEQ ID NO: 178, a LCDR2 comprising or consisting of SEQ ID NO: 68, and a LCDR3 comprising or consisting of SEQ ID NO: 82;

(35) the immunoglobulin heavy chain polypeptide comprises a HCDR1 comprising or consisting of SEQ ID NO: 5, a HCDR2 comprising or consisting of SEQ ID NO: 171, and a HCDR3 comprising or consisting of SEQ ID NO: 39; and/or the immunoglobulin light chain polypeptide comprises a LCDR1 comprising or consisting of SEQ ID NO: 179, a LCDR2 comprising or consisting of SEQ ID NO: 68, and a LCDR3 comprising or consisting of SEQ ID NO: 82;

(36) the immunoglobulin heavy chain polypeptide comprises a HCDR1 comprising or consisting of SEQ ID NO: 5, a HCDR2 comprising or consisting of SEQ ID NO: 171, and a HCDR3 comprising or consisting of SEQ ID NO: 39; and/or the immunoglobulin light chain polypeptide comprises a LCDR1 comprising or consisting of SEQ ID NO: 180, a LCDR2 comprising or consisting of SEQ ID NO: 68, and a LCDR3 comprising or consisting of SEQ ID NO: 82;

(37) the immunoglobulin heavy chain polypeptide comprises a HCDR1 comprising or consisting of SEQ ID NO: 5, a HCDR2 comprising or consisting of SEQ ID NO: 171, and a HCDR3 comprising or consisting of SEQ ID NO: 39; and/or the immunoglobulin light chain polypeptide comprises a LCDR1 comprising or consisting of SEQ ID NO: 181, a LCDR2 comprising or consisting of SEQ ID NO: 68, and a LCDR3 comprising or consisting of SEQ ID NO: 82;

(38) the immunoglobulin heavy chain polypeptide comprises a HCDR1 comprising or consisting of SEQ ID NO: 5, a HCDR2 comprising or consisting of SEQ ID NO: 171, and a HCDR3 comprising or consisting of SEQ ID NO: 39; and/or the immunoglobulin light chain polypeptide comprises a LCDR1 comprising or consisting of SEQ ID NO: 182, a LCDR2 comprising or consisting of SEQ ID NO: 68, and a LCDR3 comprising or consisting of SEQ ID NO: 82;

(39) the immunoglobulin heavy chain polypeptide comprises a HCDR1 comprising or consisting of SEQ ID NO: 5, a HCDR2 comprising or consisting of SEQ ID NO: 171, and a HCDR3 comprising or consisting of SEQ ID NO: 39; and/or the immunoglobulin light chain polypeptide comprises a LCDR1 comprising or consisting of SEQ ID NO: 53, a LCDR2 comprising or consisting of SEQ ID NO: 68, and a LCDR3 comprising or consisting of SEQ ID NO: 82;

(40) the immunoglobulin heavy chain polypeptide comprises a HCDR1 comprising or consisting of SEQ ID NO: 5, a HCDR2 comprising or consisting of SEQ ID NO: 171, and a HCDR3 comprising or consisting of SEQ ID NO: 39; and/or the immunoglobulin light chain polypeptide comprises a LCDR1 comprising or consisting of SEQ ID NO: 58, a LCDR2 comprising or consisting of SEQ ID NO: 68, and a LCDR3 comprising or consisting of SEQ ID NO: 82;

(41) the immunoglobulin heavy chain polypeptide comprises a HCDR1 comprising or consisting of SEQ ID NO: 5, a HCDR2 comprising or consisting of SEQ ID NO: 171, and a HCDR3 comprising or consisting of SEQ ID NO: 39; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(42) the immunoglobulin heavy chain polypeptide comprises a HCDR1 comprising or consisting of SEQ ID NO: 5, a HCDR2 comprising or consisting of SEQ ID NO: 171, and a HCDR3 comprising or consisting of SEQ ID NO: 39; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 80;

(43) the immunoglobulin heavy chain polypeptide comprises a HCDR1 comprising or consisting of SEQ ID NO: 5, a HCDR2 comprising or consisting of SEQ ID NO: 171, and a HCDR3 comprising or consisting of SEQ ID NO: 39; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 80;

(44) the immunoglobulin heavy chain polypeptide comprises a HCDR1 comprising or consisting of SEQ ID NO: 5, a HCDR2 comprising or consisting of SEQ ID NO: 171, and a HCDR3 comprising or consisting of SEQ ID NO: 39; and/or the immunoglobulin light chain polypeptide comprises a LCDR1 comprising or consisting of SEQ ID NO: 63, a LCDR2 comprising or consisting of SEQ ID NO: 68, and a LCDR3 comprising or consisting of SEQ ID NO: 82;

(45) the immunoglobulin heavy chain polypeptide comprises a HCDR1 comprising or consisting of SEQ ID NO: 3, a HCDR2 comprising or consisting of SEQ ID NO: 203, and a HCDR3 comprising or consisting of SEQ ID NO: 34; and/or the immunoglobulin light chain polypeptide comprises a LCDR1 comprising or consisting of SEQ ID NO: 53, a LCDR2 comprising or consisting of SEQ ID NO: 68, and a LCDR3 comprising or consisting of SEQ ID NO: 80;

(46) the immunoglobulin heavy chain polypeptide comprises a HCDR1 comprising or consisting of SEQ ID NO: 5, a HCDR2 comprising or consisting of SEQ ID NO: 204, and a HCDR3 comprising or consisting of SEQ ID NO: 36; and/or the immunoglobulin light chain polypeptide comprises a LCDR1 comprising or consisting of SEQ ID NO: 53, a LCDR2 comprising or consisting of SEQ ID NO: 71, and a LCDR3 comprising or consisting of SEQ ID NO: 80;

(47) the immunoglobulin heavy chain polypeptide comprises a HCDR1 comprising or consisting of SEQ ID NO: 2, a HCDR2 comprising or consisting of SEQ ID NO: 205, and a HCDR3 comprising or consisting of SEQ ID NO: 41; and/or the immunoglobulin light chain polypeptide comprises a LCDR1 comprising or consisting of SEQ ID NO: 53, a LCDR2 comprising or consisting of SEQ ID NO: 74, and a LCDR3 comprising or consisting of SEQ ID NO: 85;

(48) the immunoglobulin heavy chain polypeptide comprises a HCDR1 comprising or consisting of SEQ ID NO: 3, a HCDR2 comprising or consisting of SEQ ID NO: 169, and a HCDR3 comprising or consisting of SEQ ID NO: 207; and/or the immunoglobulin light chain polypeptide comprises a LCDR1 comprising or consisting of SEQ ID NO: 57, a LCDR2 comprising or consisting of SEQ ID NO: 73, and a LCDR3 comprising or consisting of SEQ ID NO: 84;

(49) the immunoglobulin heavy chain polypeptide comprises a HCDR1 comprising or consisting of SEQ ID NO: 7, a HCDR2 comprising or consisting of SEQ ID NO: 206, and a HCDR3 comprising or consisting of SEQ ID NO: 208; and/or the immunoglobulin light chain polypeptide comprises a LCDR1 comprising or consisting of SEQ ID NO: 59, a LCDR2 comprising or consisting of SEQ ID NO: 68, and a LCDR3 comprising or consisting of SEQ ID NO: 82; and/or

(50) the immunoglobulin heavy chain polypeptide and light chain polypeptide comprises any combination of the CDRs listed in Table 1.

In particular embodiments, the binding agent comprises an immunoglobulin heavy chain polypeptide and an immunoglobulin light chain polypeptide, wherein the immunoglobulin heavy chain polypeptide comprises a first framework region, a second framework region, a third framework region, and/or a fourth framework region; and/or the immunoglobulin light chain polypeptide comprises a first framework region, a second framework region, a third framework region, and/or a fourth framework region; and/or the immunoglobulin heavy chain polypeptide and light chain polypeptide comprises any combination of the framework regions listed in Tables 2 and/or 3.

TABLE 1

| Binding | SEQ | | SEQ | | SEQ | | SEQ | | SEQ | | SEQ | |
|---|---|---|---|---|---|---|---|---|---|---|---|---|
| agent | ID | HCDR1 | ID | HCDR2 | ID | HCDR3 | ID | LCDR1 | ID | LCDR2 | ID | LCDR3 |
| 1 | 1 | SDYMH | 15 | WMSPYNGIT | 32 | DRFSGSY | 53 | RASQSISS | 68 | AASSLQ | 80 | QQSYSTPY |
| | | | | GYAQKFQG | | DY | | WLA | | S | | T |
| 2 | 2 | GYYMH | 16 | WMSPSSGIT | 33 | DRGWFDP | 54 | RASQSVGT | 69 | AASTLE | 81 | QQSFSTPY |
| | | | | GYAQKFQG | | | | WLA | | N | | T |
| 3 | 3 | SYYMH | 17 | WMTTNSGIT | 34 | EGYSSGL | 53 | RASQSISS | 68 | AASSLQ | 80 | QQSYSTPY |
| | | | | GYAQKFQG | | DY | | WLA | | S | | T |
| 4 | 4 | GYYIH | 18 | GIIPIFGTA | 35 | DGRFWSG | 55 | RASQGISN | 70 | RASNLE | 82 | QQSYSTPL |
| | | | | SYAQKFQG | | YPDY | | YLA | | S | | T |
| 5 | 5 | THYMH | 19 | WMNPNSGH | 36 | ESIAVAG | 53 | RASQSISS | 71 | AASTLQ | 80 | QQSYSTPY |
| | | | | AGSAQKFQG | | YDY | | WLA | | R | | T |
| 6 | 6 | SHDIN | 20 | WMNPNSGNT | 37 | DRWYMGS | 56 | RASQSIST | 72 | AASTLQ | 83 | QQSYSTPF |
| | | | | GYAQKFQG | | ADY | | WLA | | S | | T |
| 7 | 3 | SYYMH | 20 | WMNPNSGNT | 38 | DDWGGDW | 57 | QASQDISN | 73 | GASNLQ | 84 | QQSYSTPI |
| | | | | GYAQKFQG | | FDP | | HLN | | R | | T |
| 8 | 5 | THYMH | 21 | WMNPNSGNT | 39 | ERLSVAG | 58 | RASQGISS | 68 | AASSLQ | 80 | QQSYSTPY |
| | | | | GYSQKFQG | | FDY | | WLA | | S | | T |
| 9 | 7 | DHYLH | 22 | WMNPNIGNT | 40 | EPLQLGG | 59 | RASESISS | 68 | AASSLQ | 82 | QQSYSTPL |
| | | | | GYAQKFQG | | FDY | | WLA | | S | | T |
| 10 | 2 | GYYMH | 23 | WMNPNGGTT | 41 | EGFGPNA | 53 | RASQSISS | 74 | AASNLQ | 85 | QQYYSTPY |
| | | | | GYAQNFQG | | FDI | | WLA | | S | | T |
| 11 | 2 | GYYMH | 20 | WMNPNSGNT | 42 | DSWYGDW | 60 | RASQSVGS | 75 | GASSLQ | 82 | QQSYSTPL |
| | | | | GYAQKFQG | | FDP | | WLA | | S | | T |
| 12 | 2 | GYYMH | 15 | WMSPYNGIT | 43 | EVIEVGM | 53 | RASQSISS | 76 | AASHLQ | 80 | QQSYSTPY |
| | | | | GYAQKFQG | | DV | | WLA | | S | | T |
| 13 | 8 | NYYMH | 16 | WMSPSSGIT | 44 | EAWFGEL | 61 | RASQNISN | 68 | AASSLQ | 86 | QQSYSLPY |
| | | | | GYAQKFQG | | ST | | FLN | | S | | T |
| 14 | 9 | AYYVH | 24 | WMNPNRGIT | 45 | EAYVAAF | 58 | RASQGISS | 68 | AASSLQ | 82 | QQSYSTPL |
| | | | | DSAQKFQG | | DI | | WLA | | S | | T |
| 15 | 10 | RHYVH | 25 | WMNPNSGSA | 46 | ERGYNAF | 62 | RASQSLSS | 77 | GASTRA | 87 | HQYFTTPL |
| | | | | GYAQKFQG | | DY | | SYLA | | T | | T |
| 16 | 11 | NYIH | 26 | WIHPRSGAT | 47 | DSVFGLD | 63 | RASQSISS | 72 | AASTLQ | 88 | QQSYSMPY |
| | | | | GYAPKFQG | | Y | | YLN | | S | | T |
| 17 | 3 | SYYMH | 27 | WISPRSGVT | 48 | DLDYVRA | 64 | RASQSISR | 68 | AASSLQ | 80 | QQSYSTPY |
| | | | | SYAQKFQG | | FDI | | WLA | | S | | T |
| 18 | 3 | SYYMH | 28 | WMDPNSGNT | 49 | ESWGGYF | 53 | RASQSISS | 78 | DSSSLQ | 89 | QQSYSTPV |
| | | | | GYAQKFQG | | DL | | WLA | | T | | T |
| 19 | 12 | NHYVH | 29 | WMNPTGGIT | 50 | DRTTYAF | 65 | RDSHSITT | 79 | AASNLE | 90 | QHFYNTQY |
| | | | | GYAQKFQG | | DI | | WLA | | S | | T |
| 20 | 13 | SHYMH | 30 | WMNPNSGHT | 51 | GNWVDAF | 66 | RASQVIRN | 72 | AASTLQ | 91 | QQSLQYPS |
| | | | | GNAQKFQG | | DI | | DLA | | S | | HF |
| 21 | 14 | RHLLH | 31 | WVSPIHGLT | 52 | VHGSGSD | 67 | RASQSISR | 72 | AASTLQ | 82 | QQSYSTPL |
| | | | | GYAPRFQG | | GMDV | | YLN | | S | | T |
| 22 | 167 | NHYMH | 168 | WMNPNSGHT | 174 | EAVAGPM | 63 | RASQSISS | 68 | AASSLQ | 82 | QQSYSTPL |
| | | | | GYAQRFQG | | DV | | YLN | | S | | T |
| 23 | 13 | SHYMH | 169 | WINPNSGNT | 175 | ERFLGGX | 63 | RASQSISS | 68 | AASSLQ | 82 | QQSYSTPL |
| | | | | GYAQKFQG | | MDV | | YLN | | S | | T |
| 24 | 167 | NHYMH | 170 | WINPNSGHT | 176 | EAVAGPX | 63 | RASQSISS | 68 | AASSLQ | 82 | QQSYSTPL |
| | | | | GYAQKFQG | | MDV | | YLN | | S | | T |
| 25 | 167 | NHYMH | 169 | WINPNSGNT | 177 | DRWGGDY | 63 | RASQSISS | 68 | AASSLQ | 82 | QQSYSTPL |
| | | | | GYAQKFQG | | YSA | | YLN | | S | | T |
| 26 | 5 | THYMH | 171 | WINPNSGNT | 39 | ERLSVAG | 63 | RASQSISS | 68 | AASSLQ | 82 | QQSYSTPL |
| | | | | GYSQKFQG | | FDY | | YLN | | S | | T |
| 27 | 167 | NHYMH | 168 | WMNPNSGHT | 174 | EAVAGPM | 63 | RASQSISS | 68 | AASSLQ | 82 | QQSYSTPL |
| | | | | GYAQRFQG | | DV | | YLN | | S | | T |
| 28 | 167 | NHYMH | 168 | WMNPNSGHT | 174 | EAVAGPM | 63 | RASQSISS | 68 | AASSLQ | 82 | QQSYSTPL |
| | | | | GYAQRFQG | | DV | | YLN | | S | | T |
| 29 | 167 | NHYMH | 172 | WMNPNSGHT | 174 | EAVAGPM | 63 | RASQSISS | 68 | AASSLQ | 82 | QQSYSTPL |
| | | | | GYAQKFQG | | DV | | YLN | | S | | T |
| 30 | 167 | NHYMH | 173 | WINPNSGHT | 174 | EAVAGPM | 63 | RASQSISS | 68 | AASSLQ | 82 | QQSYSTPL |
| | | | | GYAQRFQG | | DV | | YLN | | S | | T |
| 31 | 167 | NHYMH | 172 | WMNPNSGHT | 174 | EAVAGPM | 63 | RASQSISS | 68 | AASSLQ | 82 | QQSYSTPL |
| | | | | GYAQKFQG | | DV | | YLN | | S | | T |
| 32 | 167 | NHYMH | 170 | WINPNSGHT | 174 | EAVAGPM | 63 | RASQSISS | 68 | AASSLQ | 82 | QQSYSTPL |
| | | | | GYAQKFQG | | DV | | YLN | | S | | T |
| 33 | 5 | THYMH | 21 | WMNPNSGNT | 39 | ERLSVAG | 63 | RASQSISS | 68 | AASSLQ | 82 | QQSYSTPL |
| | | | | GYSQKFQG | | FDY | | YLN | | S | | T |
| 34 | 5 | THYMH | 171 | WINPNSGNT | 39 | ERLSVAG | 178 | RASQGISS | 68 | AASSLQ | 82 | QQSYSTPL |
| | | | | GYSQKFQG | | FDY | | YLN | | S | | T |
| 35 | 5 | THYMH | 171 | WINPNSGNT | 39 | ERLSVAG | 179 | RASQSISS | 68 | AASSLQ | 82 | QQSYSTPL |
| | | | | GYSQKFQG | | FDY | | WLN | | S | | T |
| 36 | 5 | THYMH | 171 | WINPNSGNT | 39 | ERLSVAG | 180 | RASQSISS | 68 | AASSLQ | 82 | QQSYSTPL |
| | | | | GYSQKFQG | | FDY | | YLA | | S | | T |
| 37 | 5 | THYMH | 171 | WINPNSGNT | 39 | ERLSVAG | 181 | RASQGISS | 68 | AASSLQ | 82 | QQSYSTPL |
| | | | | GYSQKFQG | | FDY | | WLN | | S | | T |
| 38 | 5 | THYMH | 171 | WINPNSGNT | 39 | ERLSVAG | 182 | RASQGISS | 68 | AASSLQ | 82 | QQSYSTPL |
| | | | | GYSQKFQG | | FDY | | YLA | | S | | T |
| 39 | 5 | THYMH | 171 | WINPNSGNT | 39 | ERLSVAG | 53 | RASQSISS | 68 | AASSLQ | 82 | QQSYSTPL |
| | | | | GYSQKFQG | | FDY | | WLA | | S | | T |
| 40 | 5 | THYMH | 171 | WINPNSGNT | 39 | ERLSVAG | 58 | RASQGISS | 68 | AASSLQ | 82 | QQSYSTPL |
| | | | | GYSQKFQG | | FDY | | WLA | | S | | T |
| 41 | 5 | THYMH | 171 | WINPNSGNT | 39 | ERLSVAG | 63 | RASQSISS | 68 | AASSLQ | 82 | QQSYSTPL |
| | | | | GYSQKFQG | | FDY | | YLN | | S | | T |
| 42 | 5 | THYMH | 171 | WINPNSGNT | 39 | ERLSVAG | 63 | RASQSISS | 68 | AASSLQ | 80 | QQSYSTPY |
| | | | | GYSQKFQG | | FDY | | YLN | | S | | T |
| 43 | 5 | THYMH | 171 | WINPNSGNT | 39 | ERLSVAG | 63 | RASQSISS | 68 | AASSLQ | 80 | QQSYSTPY |
| | | | | GYSQKFQG | | FDY | | YLN | | S | | T |
| 44 | 5 | THYMH | 171 | WINPNSGNT | 39 | ERLSVAG | 63 | RASQSISS | 68 | AASSLQ | 82 | QQSYSTPL |
| | | | | GYSQKFQG | | FDY | | YLN | | S | | T |
| 45 | 3 | SYYMH | 203 | WITTNSGIT | 34 | EGYSSGL | 53 | RASQSISS | 68 | AASSLQ | 80 | QQSYSTPY |
| | | | | GYAQKFQG | | DY | | WLA | | S | | T |
| 46 | 5 | THYMH | 204 | WINPNSGHA | 36 | ESIAVAG | 53 | RASQSISS | 71 | AASTLQ | 80 | QQSYSTPY |
| | | | | GSAQKFQG | | YDY | | WLA | | R | | T |
| 47 | 2 | GYYMH | 205 | WINPNSGTT | 41 | EGFGPNA | 53 | RASQSISS | 74 | AASNLQ | 85 | QQYYSTPY |
| | | | | GYAQNFQG | | FDI | | WLA | | S | | T |
| 48 | 3 | SYYMH | 169 | WINPNSGNT | 207 | DDWGGDW | 57 | QASQDISN | 73 | GASNLQ | 84 | QQSYSTPI |
| | | | | GYAQKFQG | | FDY | | HLN | | R | | T |
| 49 | 7 | DHYLH | 206 | WINPNIGNT | 208 | EPLQLGG | 59 | RASESISS | 68 | AASSLQ | 82 | QQSYSTPL |
| | | | | GYAQKFQG | | FDY | | WLA | | S | | T |

TABLE 2

| Binding | SEQ | | SEQ | | SEQ | | SEQ | |
|---|---|---|---|---|---|---|---|---|
| agent | ID | HFW1 | ID | HFW2 | ID | HFW3 | ID | HFW4 |
| 1 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 2 | 93 | QVQLVQSGAEVKKPGA | 100 | WVRQAPGQG | 102 | RVTMTRDTSTSTVYMELSS | 104 | WGQGTLV |
| | | SVKVSCKASGYTFT | | LEWMG | | LRSEDTAVYYCAR | | TVSS |
| 3 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 4 | 94 | QVQLVQSGAEVKKPGS | 100 | WVRQAPGQG | 103 | RVTITADESTSTAYMELSS | 104 | WGQGTLV |
| | | SVKVSCKASGYTFT | | LEWMG | | LRSEDTAVYYCAR | | TVSS |
| 5 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 6 | 93 | QVQLVQSGAEVKKPGA | 100 | WVRQAPGQG | 102 | RVTMTRDTSTSTVYMELSS | 104 | WGQGTLV |
| | | SVKVSCKASGYTFT | | LEWMG | | LRSEDTAVYYCAR | | TVSS |
| 7 | 93 | QVQLVQSGAEVKKPGA | 100 | WVRQAPGQG | 102 | RVTMTRDTSTSTVYMELSS | 104 | WGQGTLV |
| | | SVKVSCKASGYTFT | | LEWMG | | LRSEDTAVYYCAR | | TVSS |
| 8 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 9 | 95 | QVQLVQSGAEVKEPGA | 100 | WVRQAPGQG | 102 | RVTMTRDTSTSTVYMELSS | 104 | WGQGTLV |
| | | SVKVSCKASGYTFT | | LEWMG | | LRSEDTAVYYCAR | | TVSS |
| 10 | 93 | QVQLVQSGAEVKKPGA | 100 | WVRQAPGQG | 102 | RVTMTRDTSTSTVYMELSS | 105 | WGQGTTV |
| | | SVKVSCKASGYTFT | | LEWMG | | LRSEDTAVYYCAR | | TVSS |
| 11 | 93 | QVQLVQSGAEVKKPGA | 100 | WVRQAPGQG | 102 | RVTMTRDTSTSTVYMELSS | 104 | WGQGTLV |
| | | SVKVSCKASGYTFT | | LEWMG | | LRSEDTAVYYCAR | | TVSS |
| 12 | 93 | QVQLVQSGAEVKKPGA | 101 | WMRQAPGQG | 102 | RVTMTRDTSTSTVYMELSS | 105 | WGQGTTV |
| | | SVKVSCKASGYTFT | | LEWIG | | LRSEDTAVYYCAR | | TVSS |
| 13 | 93 | QVQLVQSGAEVKKPGA | 100 | WVRQAPGQG | 102 | RVTMTRDTSTSTVYMELSS | 104 | WGQGTLV |
| | | SVKVSCKASGYTFT | | LEWMG | | LRSEDTAVYYCAR | | TVSS |
| 14 | 96 | QVQLVQSGAEVKKPGA | 100 | WVRQAPGQG | 102 | RVTMTRDTSTSTVYMELSS | 105 | WGQGTTV |
| | | SVKVSCKASGYNFS | | LEWMG | | LRSEDTAVYYCAR | | TVSS |
| 15 | 93 | QVQLVQSGAEVKKPGA | 100 | WVRQAPGQG | 102 | RVTMTRDTSTSTVYMELSS | 104 | WGQGTLV |
| | | SVKVSCKASGYTFT | | LEWMG | | LRSEDTAVYYCAR | | TVSS |
| 16 | 97 | QVQLVQSGAEVKKPGA | 100 | WVRQAPGQG | 102 | RVTMTRDTSTSTVYMELSS | 104 | WGQGTLV |
| | | SVKVSCKASGYTLP | | LEWMG | | LRSEDTAVYYCAR | | TVSS |
| 17 | 93 | QVQLVQSGAEVKKPGA | 100 | WVRQAPGQG | 102 | RVTMTRDTSTSTVYMELSS | 105 | WGQGTTV |
| | | SVKVSCKASGYTFT | | LEWMG | | LRSEDTAVYYCAR | | TVSS |
| 18 | 98 | QVQLVQSGAEVKKPGA | 100 | WVRQAPGQG | 102 | RVTMTRDTSTSTVYMELSS | 106 | WGRGTLV |
| | | SVKVSCKASGYSFT | | LEWMG | | LRSEDTAVYYCAR | | TVSS |
| 19 | 99 | QVQLVQSGAEVKKPGA | 100 | WVRQAPGQG | 102 | RVTMTRDTSTSTVYMELSS | 107 | WGQGTMV |
| | | SVKVSCKASGYTFI | | LEWMG | | LRSEDTAVYYCAR | | TVSS |
| 20 | 93 | QVQLVQSGAEVKKPGA | 100 | WVRQAPGQG | 102 | RVTMTRDTSTSTVYMELSS | 107 | WGQGTMV |
| | | SVKVSCKASGYTFT | | LEWMG | | LRSEDTAVYYCAR | | TVSS |
| 21 | 94 | QVQLVQSGAEVKKPGS | 100 | WVRQAPGQG | 103 | RVTITADESTSTAYMELSS | 105 | WGQGTTV |
| | | SVKVSCKASGYTFT | | LEWMG | | LRSEDTAVYYCAR | | TVSS |
| 22 | 183 | QVQLVQSGAEVKKPGA | 100 | WVRQAPGQG | 102 | RVTMTRDTSTSTVYMELSS | 105 | WGQGTTV |
| | | SVKVSCKASGDTFT | | LEWMG | | LRSEDTAVYYCAR | | TVSS |
| 23 | 93 | QVQLVQSGAEVKKPGA | 100 | WVRQAPGQG | 102 | RVTMTRDTSTSTVYMELSS | 105 | WGQGTTV |
| | | SVKVSCKASGYTFT | | LEWMG | | LRSEDTAVYYCAR | | TVSS |
| 24 | 93 | QVQLVQSGAEVKKPGA | 100 | WVRQAPGQG | 102 | RVTMTRDTSTSTVYMELSS | 105 | WGQGTTV |
| | | SVKVSCKASGYTFT | | LEWMG | | LRSEDTAVYYCAR | | TVSS |
| 25 | 93 | QVQLVQSGAEVKKPGA | 100 | WVRQAPGQG | 102 | RVTMTRDTSTSTVYMELSS | 104 | WGQGTLV |
| | | SVKVSCKASGYTFT | | LEWMG | | LRSEDTAVYYCAR | | TVSS |
| 26 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 27 | 93 | QVQLVQSGAEVKKPGA | 100 | WVRQAPGQG | 102 | RVTMTRDTSTSTVYMELSS | 105 | WGQGTTV |
| | | SVKVSCKASGYTFT | | LEWMG | | LRSEDTAVYYCAR | | TVSS |
| 28 | 93 | QVQLVQSGAEVKKPGA | 100 | WVRQAPGQG | 102 | RVTMTRDTSTSTVYMELSS | 105 | WGQGTTV |
| | | SVKVSCKASGYTFT | | LEWMG | | LRSEDTAVYYCAR | | TVSS |
| 29 | 183 | QVQLVQSGAEVKKPGA | 100 | WVRQAPGQG | 102 | RVTMTRDTSTSTVYMELSS | 105 | WGQGTTV |
| | | SVKVSCKASGDTFT | | LEWMG | | LRSEDTAVYYCAR | | TVSS |
| 30 | 93 | QVQLVQSGAEVKKPGA | 100 | WVRQAPGQG | 102 | RVTMTRDTSTSTVYMELSS | 105 | WGQGTTV |
| | | SVKVSCKASGYTFT | | LEWMG | | LRSEDTAVYYCAR | | TVSS |
| 31 | 93 | QVQLVQSGAEVKKPGA | 100 | WVRQAPGQG | 102 | RVTMTRDTSTSTVYMELSS | 105 | WGQGTTV |
| | | SVKVSCKASGYTFT | | LEWMG | | LRSEDTAVYYCAR | | TVSS |
| 32 | 183 | QVQLVQSGAEVKKPGA | 100 | WVRQAPGQG | 102 | RVTMTRDTSTSTVYMELSS | 105 | WGQGTTV |
| | | SVKVSCKASGDTFT | | LEWMG | | LRSEDTAVYYCAR | | TVSS |
| 33 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 34 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 35 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 36 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 37 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 38 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 39 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 40 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 41 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 42 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 43 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 44 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 45 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 46 | 92 | QVQLVQSGAEVKKPGA | 100 | WVRQAPGQG | 102 | RVTMTRDTSTSTVYMELSS | 104 | WGQGTLV |
| | | SVKVSCKASGYTFS | | LEWMG | | LRSEDTAVYYCAR | | TVSS |
| 47 | 93 | QVQLVQSGAEVKKPGA | 100 | WVRQAPGQG | 102 | RVTMTRDTSTSTVYMELSS | 105 | WGQGTTV |
| | | SVKVSCKASGYTFT | | LEWMG | | LRSEDTAVYYCAR | | TVSS |
| 48 | 93 | QVQLVQSGAEVKKPGA | 100 | WVRQAPGQG | 102 | RVTMTRDTSTSTVYMELSS | 104 | WGQGTLV |
| | | SVKVSCKASGYTFT | | LEWMG | | LRSEDTAVYYCAR | | TVSS |
| 49 | 93 | QVQLVQSGAEVKKPGA | 100 | WVRQAPGQG | 102 | RVTMTRDTSTSTVYMELSS | 104 | WGQGTLV |
| | | SVKVSCKASGYTFT | | LEWMG | | LRSEDTAVYYCAR | | TVSS |

TABLE 3

| Binding | SEQ | | SEQ | | SEQ | | SEQ | |
|---|---|---|---|---|---|---|---|---|
| agent | ID | LFW1 | ID | LFW2 | ID | LFW3 | ID | LFW4 |
| 1 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 2 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 3 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 4 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 5 | 108 | DIQMTQSPSSLSASVG | 111 | WYQQKPGKAPK | 114 | GVPSRFSGSGSGTDFTLTISSLQP | 119 | FGQGTKL |
| | | DRVTITC | | LLIY | | EDFATYYC | | EIK |
| 6 | 108 | DIQMTQSPSSLSASVG | 111 | WYQQKPGKAPK | 114 | GVPSRFSGSGSGTDFTLTISSLQP | 120 | FGQGTKV |
| | | DRVTITC | | LLIY | | EDFATYYC | | EIK |
| 7 | 108 | DIQMTQSPSSLSASVG | 111 | WYQQKPGKAPK | 114 | GVPSRFSGSGSGTDFTLTISSLQP | 121 | FGQGTRL |
| | | DRVTITC | | LLIY | | EDFATYYC | | EIK |
| 8 | 108 | DIQMTQSPSSLSASVG | 111 | WYQQKPGKAPK | 114 | GVPSRFSGSGSGTDFTLTISSLQP | 120 | FGQGTKV |
| | | DRVTITC | | LLIY | | EDFATYYC | | EIK |
| 9 | 108 | DIQMTQSPSSLSASVG | 111 | WYQQKPGKAPK | 114 | GVPSRFSGSGSGTDFTLTISSLQP | 119 | FGQGTKL |
| | | DRVTITC | | LLIY | | EDFATYYC | | EIK |
| 10 | 108 | DIQMTQSPSSLSASVG | ill | WYQQKPGKAPK | 114 | GVPSRFSGSGSGTDFTLTISSLQP | 120 | FGQGTKV |
| | | DRVTITC | | LLIY | | EDFATYYC | | EIK |
| 11 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 12 | 108 | DIQMTQSPSSLSASVG | 111 | WYQQKPGKAPK | 114 | GVPSRFSGSGSGTDFTLTISSLQP | 121 | FGQGTRL |
| | | DRVTITC | | LLIY | | EDFATYYC | | EIK |
| 13 | 108 | DIQMTQSPSSLSASVG | 111 | WYQQKPGKAPK | 114 | GVPSRFSGSGSGTDFTLTISSLQP | 120 | FGQGTKV |
| | | DRVTITC | | LLIY | | EDFATYYC | | EIK |
| 14 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 15 | 109 | EIVMTQSPATLSVSPG | 112 | WYQQKPGQAPR | 115 | GIPARFSGSGSGTEFTLTISSLQS | 121 | FGQGTRL |
| | | ERATLSC | | LLIY | | EDFAVYYC | | EIK |
| 16 | 108 | DIQMTQSPSSLSASVG | 111 | WYQQKPGKAPK | 114 | GVPSRFSGSGSGTDFTLTISSLQP | 119 | FGQGTKL |
| | | DRVTITC | | LLIY | | EDFATYYC | | EIK |
| 17 | 108 | DIQMTQSPSSLSASVG | 111 | WYQQKPGKAPK | 114 | GVPSRFSGSGSGTDFTLTISSLQP | 119 | FGQGTKL |
| | | DRVTITC | | LLIY | | EDFATYYC | | EIK |
| 18 | 108 | DIQMTQSPSSLSASVG | 111 | WYQQKPGKAPK | 114 | GVPSRFSGSGSGTDFTLTISSLQP | 120 | FGQGTKV |
| | | DRVTITC | | LLIY | | EDFATYYC | | EIK |
| 19 | 110 | DIQITHSPSSLSASVG | 113 | WYHQKPWNAPK | 116 | GVPSRFSGSGSGTYFTLTISSLQP | 120 | FGQGTKV |
| | | YRLTITC | | LMIY | | EDFATYYC | | EIK |
| 20 | 108 | DIQMTQSPSSLSASVG | 111 | WYQQKPGKAPK | 114 | GVPSRFSGSGSGTDFTLTISSLQP | 120 | FGQGTKV |
| | | DRVTITC | | LLIY | | EDFATYYC | | EIK |
| 21 | 108 | DIQMTQSPSSLSASVG | 111 | WYQQKPGKAPK | 117 | GVPSRFSGSGSGTDFTLTISSLQP | 122 | FGPGTKV |
| | | DRVTITC | | LLIY | | EDFAPYYC | | DIK |
| 22 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 23 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 24 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 25 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 26 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 27 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 28 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 29 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 30 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 31 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 32 | 108 | DIQMTQSPSSLSASVG | ill | WYQQKPGKAPK | 114 | GVPSRFSGSGSGTDFTLTISSLQP | 118 | FGGGTKV |
| | | DRVTITC | | LLIY | | EDFATYYC | | EIK |
| 33 | 108 | DIQMTQSPSSLSASVG | ill | WYQQKPGKAPK | 114 | GVPSRFSGSGSGTDFTLTISSLQP | 118 | FGGGTKV |
| | | DRVTITC | | LLIY | | EDFATYYC | | EIK |
| 34 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 35 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 36 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 37 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 38 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 39 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 40 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 41 | 108 | DIQMTQSPSSLSASVG | 111 | WYQQKPGKAPK | 114 | GVPSRFSGSGSGTDFTLTISSLQP | 120 | FGQGTKV |
| | | DRVTITC | | LLIY | | EDFATYYC | | EIK |
| 42 | 108 | DIQMTQSPSSLSASVG | 111 | WYQQKPGKAPK | 114 | GVPSRFSGSGSGTDFTLTISSLQP | 120 | FGQGTKV |
| | | DRVTITC | | LLIY | | EDFATYYC | | EIK |
| 43 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 44 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 45 | 108 | DIQMTQSPSSLSASVG | 111 | WYQQKPGKAPK | 114 | GVPSRFSGSGSGTDFTLTISSLQP | 118 | FGGGTKV |
| | | DRVTITC | | LLIY | | EDFATYYC | | EIK |
| 46 | 108 | DIQMTQSPSSLSASVG | 111 | WYQQKPGKAPK | 114 | GVPSRFSGSGSGTDFTLTISSLQP | 118 | FGQGTKL |
| | | DRVTITC | | LLIY | | EDFATYYC | | EIK |
| 47 | 108 | DIQMTQSPSSLSASVG | 111 | WYQQKPGKAPK | 114 | GVPSRFSGSGSGTDFTLTISSLQP | 118 | FGQGTRL |
| | | DRVTITC | | LLIY | | EDFATYYC | | EIK |
| 48 | 108 | DIQMTQSPSSLSASVG | 111 | WYQQKPGKAPK | 114 | GVPSRFSGSGSGTDFTLTISSLQP | 118 | FGQGTRL |
| | | DRVTITC | | LLIY | | EDFATYYC | | EIK |
| 49 | 108 | DIQMTQSPSSLSASVG | 111 | WYQQKPGKAPK | 114 | GVPSRFSGSGSGTDFTLTISSLQP | 118 | FGQGTKL |
| | | DRVTITC | | LLIY | | EDFATYYC | | EIK |

TABLE 4

| Binding | SEQ | |
|---|---|---|
| agent | ID | |
| | | VH |
| 1 | 123 | QVQLVQSGAEVKKPGASVKVSCKASGYTFSSDYMHWVRQAPGQGLEWMGWMSPYNGITGYAQKFQGRVTMTRDTSTST |
| | | VYMELSSLRSEDTAVYYCARDRFSGSYDYWGQGTLVTVSS |
| 2 | 124 | QVQLVQSGAEVKKPGASVKVSCKASGYTFTGYYMHWVRQAPGQGLEWMGWMSPSSGITGYAQKFQGRVTMTRDTSTST |
| | | VYMELSSLRSEDTAVYYCARDRGWFDPWGQGTLVTVSS |
| 3 | 125 | QVQLVQSGAEVKKPGASVKVSCKASGYTFSSYYMHWVRQAPGQGLEWMGWMTTNSGITGYAQKFQGRVTMTRDTSTST |
| | | VYMELSSLRSEDTAVYYCAREGYSSGLDYWGQGTLVTVSS |
| 4 | 126 | QVQLVQSGAEVKKPGSSVKVSCKASGYTFTGYYIHWVRQAPGQGLEWMGGIIPIFGTASYAQKFQGRVTITADESTST |
| | | AYMELSSLRSEDTAVYYCARDGRFWSGYPDYWGQGTLVTVSS |
| 5 | 127 | QVQLVQSGAEVKKPGASVKVSCKASGYTFSTHYMHWVRQAPGQGLEWMGWMNPNSGHAGSAQKFQGRVTMTRDTSTST |
| | | VYMELSSLRSEDTAVYYCARESIAVAGYDYWGQGTLVTVSS |
| 6 | 128 | QVQLVQSGAEVKKPGASVKVSCKASGYTFTSHDINWVRQAPGQGLEWMGWMNPNSGNTGYAQKFQGRVTMTRDTSTST |
| | | VYMELSSLRSEDTAVYYCARDRWYMGSADYWGQGTLVTVSS |
| 7 | 129 | QVQLVQSGAEVKKPGASVKVSCKASGYTFTSYYMHWVRQAPGQGLEWMGWMNPNSGNTGYAQKFQGRVTMTRDTSTST |
| | | VYMELSSLRSEDTAVYYCARDDWGGDWFDPWGQGTLVTVSS |
| 8 | 130 | QVQLVQSGAEVKKPGASVKVSCKASGYTFSTHYMHWVRQAPGQGLEWMGWMNPNSGNTGYSQKFQGRVTMTRDTSTST |
| | | VYMELSSLRSEDTAVYYCARERLSVAGFDYWGQGTLVTVSS |
| 9 | 131 | QVQLVQSGAEVKEPGASVKVSCKASGYTFTDHYLHWVRQAPGQGLEWMGWMNPNIGNTGYAQKFQGRVTMTRDTSTST |
| | | VYMELSSLRSEDTAVYYCAREPLQLGGFDYWGQGTLVTVSS |
| 10 | 132 | QVQLVQSGAEVKKPGASVKVSCKASGYTFTGYYMHWVRQAPGQGLEWMGWMNPNGGTTGYAQNFQGRVTMTRDTSTST |
| | | VYMELSSLRSEDTAVYYCAREGFGPNAFDIWGQGTTVTVSS |
| 11 | 133 | QVQLVQSGAEVKKPGASVKVSCKASGYTFTGYYMHWVRQAPGQGLEWMGWMNPNSGNTGYAQKFQGRVTMTRDTSTST |
| | | VYMELSSLRSEDTAVYYCARDSWYGDWFDPWGQGTLVTVSS |
| 12 | 134 | QVQLVQSGAEVKKPGASVKVSCKASGYTFTGYYMHWMRQAPGQGLEWIGWMSPYNGITGYAQKFQGRVTMTRDTSTST |
| | | VYMELSSLRSEDTAVYYCAREVIEVGMDVWGQGTTVTVSS |
| 13 | 135 | QVQLVQSGAEVKKPGASVKVSCKASGYTFTNYYMHWVRQAPGQGLEWMGWMSPSSGITGYAQKFQGRVTMTRDTSTST |
| | | VYMELSSLRSEDTAVYYCAREAWFGELSTWGQGTLVTVSS |
| 14 | 136 | QVQLVQSGAEVKKPGASVKVSCKASGYNFSAYYVHWVRQAPGQGLEWMGWMNPNRGITDSAQKFQGRVTMTRDTSTST |
| | | VYMELSSLRSEDTAVYYCAREAYVAAFDIWGQGTTVTVSS |
| 15 | 137 | QVQLVQSGAEVKKPGASVKVSCKASGYTFTRHYVHWVRQAPGQGLEWMGWMNPNSGSAGYAQKFQGRVTMTRDTSTST |
| | | VYMELSSLRSEDTAVYYCARERGYNAFDYWGQGTLVTVSS |
| 16 | 138 | QVQLVQSGAEVKKPGASVKVSCKASGYTLPNYIHWVRQAPGQGLEWMGWIHPRSGATGYAPKFQGRVTMTRDTSTSTV |
| | | YMELSSLRSEDTAVYYCARDSVFGLDYWGQGTLVTVSS |
| 17 | 139 | QVQLVQSGAEVKKPGASVKVSCKASGYTFTSYYMHWVRQAPGQGLEWMGWISPRSGVTSYAQKFQGRVTMTRDTSTST |
| | | VYMELSSLRSEDTAVYYCARDLDYVRAFDIWGQGTTVTVSS |
| 18 | 140 | QVQLVQSGAEVKKPGASVKVSCKASGYSFTSYYMHWVRQAPGQGLEWMGWMDPNSGNTGYAQKFQGRVTMTRDTSTST |
| | | VYMELSSLRSEDTAVYYCARESWGGYFDLWGRGTLVTVSS |
| 19 | 141 | QVQLVQSGAEVKKPGASVKVSCKASGYTFINHYVHWVRQAPGQGLEWMGWMNPTGGITGYAQKFQGRVTMTRDTSTST |
| | | VYMELSSLRSEDTAVYYCARDRTTYAFDIWGQGTMVTVSS |
| 20 | 142 | QVQLVQSGAEVKKPGASVKVSCKASGYTFTSHYMHWVRQAPGQGLEWMGWMNPNSGHTGNAQKFQGRVTMTRDTSTST |
| | | VYMELSSLRSEDTAVYYCARGNWVDAFDIWGQGTMVTVSS |
| 21 | 143 | QVQLVQSGAEVKKPGSSVKVSCKASGYTFTRHLLHWVRQAPGQGLEWMGWVSPIHGLTGYAPRFQGRVTITADESTST |
| | | AYMELSSLRSEDTAVYYCARVHGSGSDGMDVWGQGTTVTVSS |
| 22 | 184 | QVQLVQSGAEVKKPGASVKVSCKASGDTFTNHYMHWVRQAPGQGLEWMGWMNPNSGHTGYAQRFQGRVTMTRDTSTST |
| | | VYMELSSLRSEDTAVYYCAREAVAGPMDVWGQGTTVTVSS |
| 23 | 185 | QVQLVQSGAEVKKPGASVKVSCKASGYTFTSHYMHWVRQAPGQGLEWMGWINPNSGNTGYAQKFQGRVTMTRDTSTST |
| | | VYMELSSLRSEDTAVYYCARERFLGGXMDVWGQGTTVTVSS |
| 24 | 186 | QVQLVQSGAEVKKPGASVKVSCKASGYTFTNHYMHWVRQAPGQGLEWMGWINPNSGHTGYAQKFQGRVTMTRDTSTST |
| | | VYMELSSLRSEDTAVYYCAREAVAGPXMDVWGQGTTVTVSS |
| 25 | 187 | QVQLVQSGAEVKKPGASVKVSCKASGYTFTNHYMHWVRQAPGQGLEWMGWINPNSGNTGYAQKFQGRVTMTRDTSTST |
| | | VYMELSSLRSEDTAVYYCARDRWGGDYYSAWGQGTLVTVSS |
| 26 | 188 | QVQLVQSGAEVKKPGASVKVSCKASGYTFSTHYMHWVRQAPGQGLEWMGWINPNSGNTGYSQKFQGRVTMTRDTSTST |
| | | VYMELSSLRSEDTAVYYCARERLSVAGFDYWGQGTLVTVSS |
| 27 | 189 | QVQLVQSGAEVKKPGASVKVSCKASGYTFTNHYMHWVRQAPGQGLEWMGWMNPNSGHTGYAQRFQGRVTMTRDTSTST |
| | | VYMELSSLRSEDTAVYYCAREAVAGPMDVWGQGTTVTVSS |
| 28 | 189 | QVQLVQSGAEVKKPGASVKVSCKASGYTFTNHYMHWVRQAPGQGLEWMGWMNPNSGHTGYAQRFQGRVTMTRDTSTST |
| | | VYMELSSLRSEDTAVYYCAREAVAGPMDVWGQGTTVTVSS |
| 29 | 190 | QVQLVQSGAEVKKPGASVKVSCKASGDTFTNHYMHWVRQAPGQGLEWMGWMNPNSGHTGYAQKFQGRVTMTRDTSTST |
| | | VYMELSSLRSEDTAVYYCAREAVAGPMDVWGQGTTVTVSS |
| 30 | 191 | QVQLVQSGAEVKKPGASVKVSCKASGYTFTNHYMHWVRQAPGQGLEWMGWINPNSGHTGYAQRFQGRVTMTRDTSTST |
| | | VYMELSSLRSEDTAVYYCAREAVAGPMDVWGQGTTVTVSS |
| 31 | 192 | QVQLVQSGAEVKKPGASVKVSCKASGYTFTNHYMHWVRQAPGQGLEWMGWMNPNSGHTGYAQKFQGRVTMTRDTSTST |
| | | VYMELSSLRSEDTAVYYCAREAVAGPMDVWGQGTTVTVSS |
| 32 | 193 | QVQLVQSGAEVKKPGASVKVSCKASGDTFTNHYMHWVRQAPGQGLEWMGWINPNSGHTGYAQKFQGRVTMTRDTSTST |
| | | VYMELSSLRSEDTAVYYCAREAVAGPMDVWGQGTTVTVSS |
| 33 | 130 | QVQLVQSGAEVKKPGASVKVSCKASGYTFSTHYMHWVRQAPGQGLEWMGWMNPNSGNTGYSQKFQGRVTMTRDTSTST |
| | | VYMELSSLRSEDTAVYYCARERLSVAGFDYWGQGTLVTVSS |
| 34 | 188 | QVQLVQSGAEVKKPGASVKVSCKASGYTFSTHYMHWVRQAPGQGLEWMGWINPNSGNTGYSQKFQGRVTMTRDTSTST |
| | | VYMELSSLRSEDTAVYYCARERLSVAGFDYWGQGTLVTVSS |
| 35 | 188 | QVQLVQSGAEVKKPGASVKVSCKASGYTFSTHYMHWVRQAPGQGLEWMGWINPNSGNTGYSQKFQGRVTMTRDTSTST |
| | | VYMELSSLRSEDTAVYYCARERLSVAGFDYWGQGTLVTVSS |
| 36 | 188 | QVQLVQSGAEVKKPGASVKVSCKASGYTFSTHYMHWVRQAPGQGLEWMGWINPNSGNTGYSQKFQGRVTMTRDTSTST |
| | | VYMELSSLRSEDTAVYYCARERLSVAGFDYWGQGTLVTVSS |
| 37 | 188 | QVQLVQSGAEVKKPGASVKVSCKASGYTFSTHYMHWVRQAPGQGLEWMGWINPNSGNTGYSQKFQGRVTMTRDTSTST |
| | | VYMELSSLRSEDTAVYYCARERLSVAGFDYWGQGTLVTVSS |
| 38 | 188 | QVQLVQSGAEVKKPGASVKVSCKASGYTFSTHYMHWVRQAPGQGLEWMGWINPNSGNTGYSQKFQGRVTMTRDTSTST |
| | | VYMELSSLRSEDTAVYYCARERLSVAGFDYWGQGTLVTVSS |
| 39 | 188 | QVQLVQSGAEVKKPGASVKVSCKASGYTFSTHYMHWVRQAPGQGLEWMGWINPNSGNTGYSQKFQGRVTMTRDTSTST |
| | | VYMELSSLRSEDTAVYYCARERLSVAGFDYWGQGTLVTVSS |
| 40 | 188 | QVQLVQSGAEVKKPGASVKVSCKASGYTFSTHYMHWVRQAPGQGLEWMGWINPNSGNTGYSQKFQGRVTMTRDTSTST |
| | | VYMELSSLRSEDTAVYYCARERLSVAGFDYWGQGTLVTVSS |
| 41 | 188 | QVQLVQSGAEVKKPGASVKVSCKASGYTFSTHYMHWVRQAPGQGLEWMGWINPNSGNTGYSQKFQGRVTMTRDTSTST |
| | | VYMELSSLRSEDTAVYYCARERLSVAGFDYWGQGTLVTVSS |
| 42 | 188 | QVQLVQSGAEVKKPGASVKVSCKASGYTFSTHYMHWVRQAPGQGLEWMGWINPNSGNTGYSQKFQGRVTMTRDTSTST |
| | | VYMELSSLRSEDTAVYYCARERLSVAGFDYWGQGTLVTVSS |
| 43 | 188 | QVQLVQSGAEVKKPGASVKVSCKASGYTFSTHYMHWVRQAPGQGLEWMGWINPNSGNTGYSQKFQGRVTMTRDTSTST |
| | | VYMELSSLRSEDTAVYYCARERLSVAGFDYWGQGTLVTVSS |
| 44 | 188 | QVQLVQSGAEVKKPGASVKVSCKASGYTFSTHYMHWVRQAPGQGLEWMGWINPNSGNTGYSQKFQGRVTMTRDTSTST |
| | | VYMELSSLRSEDTAVYYCARERLSVAGFDYWGQGTLVTVSS |
| 45 | 209 | QVQLVQSGAEVKKPGASVKVSCKASGYTFSSYYMHWVRQAPGQGLEWMGWITTNSGITGYAQKFQGRVTMTRDTSTST |
| | | VYMELSSLRSEDTAVYYCAREGYSSGLDYWGQGTLVTVSS |
| 46 | 210 | QVQLVQSGAEVKKPGASVKVSCKASGYTFSTHYMHWVRQAPGQGLEWMGWINPNSGHAGSAQKFQGRVTMTRDTSTST |
| | | VYMELSSLRSEDTAVYYCARESIAVAGYDYWGQGTLVTVSS |
| 47 | 211 | QVQLVQSGAEVKKPGASVKVSCKASGYTFTGYYMHWVRQAPGQGLEWMGWINPNSGTTGYAQNFQGRVTMTRDTSTST |
| | | VYMELSSLRSEDTAVYYCAREGFGPNAFDIWGQGTTVTVSS |
| 48 | 212 | QVQLVQSGAEVKKPGASVKVSCKASGYTFTSYYMHWVRQAPGQGLEWMGWINPNSGNTGYAQKFQGRVTMTRDTSTST |
| | | VYMELSSLRSEDTAVYYCARDDWGGDWFDYWGQGTLVTVSS |
| 49 | 213 | QVQLVQSGAEVKKPGASVKVSCKASGYTFTDHYLHWVRQAPGQGLEWMGWINPNIGNTGYAQKFQGRVTMTRDTSTST |
| | | VYMELSSLRSEDTAVYYCAREPLQLGGFDYWGQGTLVTVSS |
| | | VL |
| 1 | 144 | DIQMTQSPSSLSASVGDRVTITCRASQSISSWLAWYQQKPGKAPKLLIYAASSLQSGVPSRFSGSGSGTDFTLTISSL |
| | | QPEDFATYYCQQSYSTPYTFGGGTKVEIK |
| 2 | 145 | DIQMTQSPSSLSASVGDRVTITCRASQSVGTWLAWYQQKPGKAPKLLIYAASTLENGVPSRFSGSGSGTDFTLTISSL |
| | | QPEDFATYYCQQSFSTPYTFGGGTKVEIK |
| 3 | 146 | DIQMTQSPSSLSASVGDRVTITCRASQSISSWLAWYQQKPGKAPKLLIYAASSLQSGVPSRFSGSGSGTDFTLTISSL |
| | | QPEDFATYYCQQSYSTPYTFGGGTKVEIK |
| 4 | 147 | DIQMTQSPSSLSASVGDRVTITCRASQGISNYLAWYQQKPGKAPKLLIYRASNLESGVPSRFSGSGSGTDFTLTISSL |
| | | QPEDFATYYCQQSYSTPLTFGGGTKVEIK |
| 5 | 148 | DIQMTQSPSSLSASVGDRVTITCRASQSISSWLAWYQQKPGKAPKLLIYAASTLQRGVPSRFSGSGSGTDFTLTISSL |
| | | QPEDFATYYCQQSYSTPYTFGQGTKLEIK |
| 6 | 149 | DIQMTQSPSSLSASVGDRVTITCRASQSISTWLAWYQQKPGKAPKLLIYAASTLQSGVPSRFSGSGSGTDFTLTISSL |
| | | QPEDFATYYCQQSYSTPFTFGQGTKVEIK |
| 7 | 150 | DIQMTQSPSSLSASVGDRVTITCQASQDISNHLNWYQQKPGKAPKLLIYGASNLQRGVPSRFSGSGSGTDFTLTISSL |
| | | QPEDFATYYCQQSYSTPITFGQGTRLEIK |
| 8 | 151 | DIQMTQSPSSLSASVGDRVTITCRASQGISSWLAWYQQKPGKAPKLLIYAASSLQSGVPSRFSGSGSGTDFTLTISSL |
| | | QPEDFATYYCQQSYSTPYTFGQGTKVEIK |
| 9 | 152 | DIQMTQSPSSLSASVGDRVTITCRASESISSWLAWYQQKPGKAPKLLIYAASSLQSGVPSRFSGSGSGTDFTLTISSL |
| | | QPEDFATYYCQQSYSTPLTFGQGTKLEIK |
| 10 | 153 | DIQMTQSPSSLSASVGDRVTITCRASQSISSWLAWYQQKPGKAPKLLIYAASNLQSGVPSRFSGSGSGTDFTLTISSL |
| | | QPEDFATYYCQQYYSTPYTFGQGTKVEIK |
| 11 | 154 | DIQMTQSPSSLSASVGDRVTITCRASQSVGSWLAWYQQKPGKAPKLLIYGASSLQSGVPSRFSGSGSGTDFTLTISS |
| | | LQPEDFATYYCQQSYSTPLTFGGGTKVEIK |
| 12 | 155 | DIQMTQSPSSLSASVGDRVTITCRASQSISSWLAWYQQKPGKAPKLLIYAASHLQSGVPSRFSGSGSGTDFTLTISSL |
| | | QPEDFATYYCQQSYSTPYTFGQGTRLEIK |
| 13 | 156 | DIQMTQSPSSLSASVGDRVTITCRASQNISNFLNWYQQKPGKAPKLLIYAASSLQSGVPSRFSGSGSGTDFTLTISSL |
| | | QPEDFATYYCQQSYSLPYTFGQGTKVEIK |
| 14 | 157 | DIQMTQSPSSLSASVGDRVTITCRASQGISSWLAWYQQKPGKAPKLLIYAASSLQSGVPSRFSGSGSGTDFTLTISSL |
| | | QPEDFATYYCQQSYSTPLTFGGGTKVEIK |
| 15 | 158 | EIVMTQSPATLSVSPGERATLSCRASQSLSSSYLAWYQQKPGQAPRLLIYGASTRATGIPARFSGSGSGTEFTLTISS |
| | | LQSEDFAVYYCHQYFTTPLTFGQGTRLEIK |
| 16 | 159 | DIQMTQSPSSLSASVGDRVTITCRASQSISSYLNWYQQKPGKAPKLLIYAASTLQSGVPSRFSGSGSGTDFTLTISSL |
| | | QPEDFATYYCQQSYSMPYTFGQGTKLEIK |
| 17 | 160 | DIQMTQSPSSLSASVGDRVTITCRASQSISRWLAWYQQKPGKAPKLLIYAASSLQSGVPSRFSGSGSGTDFTLTISSL |
| | | QPEDFATYYCQQSYSTPYTFGQGTKLEIK |
| 18 | 161 | DIQMTQSPSSLSASVGDRVTITCRASQSISSWLAWYQQKPGKAPKLLIYDSSSLQTGVPSRFSGSGSGTDFTLTISSL |
| | | QPEDFATYYCQQSYSTPVTFGQGTKVEIK |
| 19 | 162 | DIQITHSPSSLSASVGYRLTITCRDSHSITTWLAWYHQKPWNAPKLMIYAASNLESGVPSRFSGSGSGTYFTLTISSL |
| | | QPEDFATYYCQHFYNTQYTFGQGTKVEIK |
| 20 | 163 | DIQMTQSPSSLSASVGDRVTITCRASQVIRNDLAWYQQKPGKAPKLLIYAASTLQSGVPSRFSGSGSGTDFTLTISSL |
| | | QPEDFATYYCQQSLQYPSHFFGQGTKVEIK |
| 21 | 164 | DIQMTQSPSSLSASVGDRVTITCRASQSISRYLNWYQQKPGKAPKLLIYAASTLQSGVPSRFSGSGSGTDFTLTISSL |
| | | QPEDFAPYYCQQSYSTPLTFGPGTKVDIK |
| 22 | 194 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGGGTKVEIK |
| 23 | 194 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGGGTKVEIK |
| 24 | 194 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGGGTKVEIK |
| 25 | 194 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGGGTKVEIK |
| 26 | 194 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGGGTKVEIK |
| 27 | 194 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGGGTKVEIK |
| 28 | 194 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGGGTKVEIK |
| 29 | 194 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGGGTKVEIK |
| 30 | 194 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGGGTKVEIK |
| 31 | 194 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGGGTKVEIK |
| 32 | 194 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGGGTKVEIK |
| 33 | 194 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGGGTKVEIK |
| 34 | 195 | DIQMTQSPSSLSASVGDRVTITCRASQGISSYLNWYQQKPGKAPKLLIYAASSLQSGVPSRFSGSGSGTDFTLTISSL |
| | | QPEDFATYYCQQSYSTPLTFGGGTKVEIK |
| 35 | 196 | DIQMTQSPSSLSASVGDRVTITCRASQSISSWLNWYQQKPGKAPKLLIYAASSLQSGVPSRFSGSGSGTDFTLTISSL |
| | | QPEDFATYYCQQSYSTPLTFGGGTKVEIK |
| 36 | 197 | DIQMTQSPSSLSASVGDRVTITCRASQSISSYLAWYQQKPGKAPKLLIYAASSLQSGVPSRFSGSGSGTDFTLTISSL |
| | | QPEDFATYYCQQSYSTPLTFGGGTKVEIK |
| 37 | 198 | DIQMTQSPSSLSASVGDRVTITCRASQGISSWLNWYQQKPGKAPKLLIYAASSLQSGVPSRFSGSGSGTDFTLTISSL |
| | | QPEDFATYYCQQSYSTPLTFGGGTKVEIK |
| 38 | 199 | DIQMTQSPSSLSASVGDRVTITCRASQGISSYLAWYQQKPGKAPKLLIYAASSLQSGVPSRFSGSGSGTDFTLTISSL |
| | | QPEDFATYYCQQSYSTPLTFGGGTKVEIK |
| 39 | 200 | DIQMTQSPSSLSASVGDRVTITCRASQSISSWLAWYQQKPGKAPKLLIYAASSLQSGVPSRFSGSGSGTDFTLTISSL |
| | | QPEDFATYYCQQSYSTPLTFGGGTKVEIK |
| 40 | 157 | DIQMTQSPSSLSASVGDRVTITCRASQGISSWLAWYQQKPGKAPKLLIYAASSLQSGVPSRFSGSGSGTDFTLTISSL |
| | | QPEDFATYYCQQSYSTPLTFGGGTKVEIK |
| 41 | 201 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGQGTKVEIK |
| 42 | 202 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPYTFGQGTKVEIK |
| 43 | 202 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPYTFGGGTKVEIK |
| 44 | 194 | DIQMTQSPSSLSASVGDRVTITCRASQSISSYLNWYQQKPGKAPKLLIYAASSLQSGVPSRFSGSGSGTDFTLTISSL |
| | | QPEDFATYYCQQSYSTPLTFGGGTKVEIK |
| 45 | 144 | DIQMTQSPSSLSASVGDRVTITCRASQSISSWLAWYQQKPGKAPKLLIYAASSLQSGVPSRFSGSGSGTDFTLTISSL |
| | | QPEDFATYYCQQSYSTPYTFGGGTKVEIK |
| 46 | 148 | DIQMTQSPSSLSASVGDRVTITCRASQSISSWLAWYQQKPGKAPKLLIYAASTLQRGVPSRFSGSGSGTDFTLTISSL |
| | | QPEDFATYYCQQSYSTPYTFGQGTKLEIK |
| 47 | 153 | DIQMTQSPSSLSASVGDRVTITCRASQSISSWLAWYQQKPGKAPKLLIYAASNLQSGVPSRFSGSGSGTDFTLTISSL |
| | | QPEDFATYYCQQYYSTPYTFGQGTKVEIK |
| 48 | 150 | DIQMTQSPSSLSASVGDRVTITCQASQDISNHLNWYQQKPGKAPKLLIYGASNLQRGVPSRFSGSGSGTDFTLTISSL |
| | | QPEDFATYYCQQSYSTPITFGQGTRLEIK |
| 49 | 152 | DIQMTQSPSSLSASVGDRVTITCRASESISSWLAWYQQKPGKAPKLLIYAASSLQSGVPSRFSGSGSGTDFTLTISSL |
| | | QPEDFATYYCQQSYSTPLTFGQGTKLEIK |

As mentioned above, the binding agent can comprise an Ig heavy and/or light variable region with at least about 90% identity (e.g. at least about 91%, at least about 92%, at least about 93%, at least about 94%, at least about 95%, at least about 96%, at least about 97%, at least about 98%, or at least about 99% identity) to a specific heavy or light chain variable region sequence provided herein. Similarly, the CDRs of the Ig heavy and/or light chain variable region can have at least about 90% identity (e.g. at least about 91%, at least about 92%, at least about 93%, at least about 94%, at least about 95%, at least about 96%, at least about 97%, at least about 98%, or at least about 99% identity) to a specific CDR sequence provided herein. Thus, the Ig heavy and light chain variable region or CDR sequence can comprise at least one (e.g., 1 or more, 2 or more, 3 or more, 4 or more, 5 or more, 6 or more, 7 or more, 8 or more, 9 or more, or 10 or more, etc., as applicable based on the length of the sequence) amino acid modification (e.g., substitution, addition, or deletion) as compared to the specific sequences provided herein, provided the binding agent maintains the ability to specifically bind PD-L1, optionally wherein the binding agent maintains the affinity of a binding agent with the specified sequences and/or competes with a binding agent having the specified sequences for binding to PD-L1.

The amino acids of the sequences provided can be substituted with any other amino acid. Amino acids include naturally-occurring α-amino acids and their stereoisomers, as well as non-naturally occurring amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypiation) or deglycosylated.

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Non-naturally occurring amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

Amino acids may be referred to herein by either the commonly known three letter symbols or by the one-letter symbols recommended by the IUPAC-IUB Biochemical Nomenclature Commission.

The amino acid substitution can be conservative, semi-conservative, or non-conservative with respect to the basic properties of the original amino acid residue. A “conservative” substitution refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (D or Asp), asparagine (N or Asn), glutamine (Q or Gln), lysine (K or Lys), and arginine (R or Arg).

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained.

“Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In addition, one or more amino acids can be inserted into the aforementioned immunoglobulin heavy or light chain variable region polypeptides. Any number of any suitable amino acids can be inserted into the amino acid sequence of the immunoglobulin heavy or light chain variable region polypeptides. In this respect, at least one amino acid (e.g., 2 or more, 5 or more, or 10 or more amino acids), but not more than 20 amino acids (e.g., 18 or less, 15 or less, or 12 or less amino acids), can be inserted into the amino acid sequence of the immunoglobulin heavy or light chain variable region polypeptides. Preferably, 1-10 amino acids (e.g., 1, 2, 3, 4, 5, 6, 7, 8, 9, or 10 amino acids) are inserted into the amino acid sequence of the immunoglobulin heavy or light chain variable region polypeptides. In this respect, the amino acid(s) can be inserted into any one of the aforementioned immunoglobulin heavy or light chain variable region polypeptides in any suitable location. In some embodiments, the amino acid(s) are inserted into a CDR (e.g., CDR1, CDR2, or CDR3) of the immunoglobulin heavy or light chain variable region polypeptides; in other embodiments, the amino acids are inserted into a framework region.

Further provided is a PD-L1 binding agent (e.g., antibody or antibody fragment) that competes with a PD-L1 binding agent (e.g., antibody or antibody fragment) having an immunoglobulin heavy and light chain variable region specifically provided herein (e.g., one of binding agents 1-21 provided herein).

The “biological activity” of an PD-L1-binding agent refers to, for example, binding affinity for PD-L1 or a particular PD-L1 epitope, neutralization or inhibition of PD-L1 protein binding to PD-1, neutralization or inhibition of PD-L1 protein activity in vivo (e.g., IC₅₀), pharmacokinetics, and cross-reactivity (e.g., with non-human homologs or orthologs of the PD-L1 protein, or with other proteins or tissues). Other biological properties or characteristics of an antigen-binding agent recognized in the art include, for example, avidity, selectivity, solubility, folding, immunotoxicity, expression, and formulation. The aforementioned properties or characteristics can be observed, measured, and/or assessed using standard techniques including, but not limited to, ELISA, competitive ELISA, surface plasmon resonance analysis (BIACORE™), or KINEXA™, in vitro or in vivo neutralization assays, receptor-ligand binding assays, cytokine or growth factor production and/or secretion assays, and signal transduction and immunohistochemistry assays.

The terms “inhibit” or “neutralize,” as used herein with respect to the activity of a PD-L1-binding agent, refer to the ability to substantially antagonize, prohibit, prevent, restrain, slow, disrupt, alter, eliminate, stop, or reverse the progression or severity of, for example, the biological activity of a PD-L1 protein, or a disease or condition associated with an PD-L1 protein. The isolated PD-L1-binding agent of the invention preferably inhibits or neutralizes the activity of a PD-L1 protein by at least about 5%, about 10%, about 15%, about 20%, about 30%, about 40%, about 50%, about 60%, about 70%, about 80%, about 90%, about 95%, about 100%, or a range defined by any two of the foregoing values.

In some embodiments, the PD-L1 binding agent (e.g., antibody or antibody fragment) exhibits antibody dependent cell-mediated cytotoxicity (ADCC). ADCC is a mechanism of cell-mediated immune defense whereby an effector cell of the immune system lyses a target cell whose membrane-surface antigens have been bound by specific antibodies. ADCC is independent of the immune complement system that also lyses targets but does not require any other cell and is part of the adaptive immune response.

In some embodiments, the PD-L1 binding agent (e.g., antibody or antibody fragment) exhibits antibody dependent cell-mediated phagocytosis (ADCP). ADCP is a cellular process by which effector cells with phagocytic potential, such as monocytes and macrophages, can internalize target cells. Once phagocytosed, the target cell resides in a phagosome, which fuses with a lysosome to begin degradation of the target cell via an oxygen-dependent or independent mechanism. This function is dependent on opsonization, or identification of the target cell with a binding agent, which then also serves as a bridge between the target cell and the phagocytic cell. Mechanistically, the binding agent binds its cognate antigen on the target cell through its antigen recognition domain, and then recruits the phagocytic cell to the target with its Fc region. Once bound to the Fc receptor of the phagocytic cell, the target cell is ingested and degraded. This process also leads to the production of soluble factors by the effector cells that help initiate and drive the immune response.

In some embodiments, the PD-L1 binding agent (e.g., antibody or antibody fragment) exhibits complement dependent cytotoxicity (CDC). CDC is an effector function of IgG and IgM antibodies. When the binding agents are bound to surface antigen, the classical complement pathway is triggered, resulting in formation of a membrane attack complex (MAC) and target cell lysis.

In some embodiments, the binding agents comprising an Fc region contain one or more modifications (e.g., amino acid insertion, deletion, and/or substitution) in the Fc region that results in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the a binding agent or antibody with the native Fc region lacking the mutation. In some embodiments, the binding agents contain one or more modifications (e.g., amino acid insertion, deletion, and/or substitution) in the Fc region that reduce the binding of the Fc region to FcγRIIB In some embodiments, the binding agents contain one or more modifications (e.g., amino acid insertion, deletion, and/or substitution) in the Fc region of the antibody that reduce the binding to FcγRIIB while maintaining the same binding or having increased binding to FcγRI (CD64), FcγRIIA (CD32A), and/or FcRγIIIA (CD16a) as compared to a binding agent or antibody with a native Fc region lacking the mutation. In some embodiments, the binding agents contain one of more modifications in the Fc region that increase the binding of the Fc region to FcγRIIB In some embodiments, the modifications substantially reduce or eliminate antibody effector functions.

The Fc region mutations can be in a CH2 domain, a CH3 domain, or a combination thereof. A “native Fc region” is synonymous with a “wild-type Fc region” and comprises an amino acid sequence that is identical to the amino acid sequence of an Fc region found in nature or identical to the amino acid sequence of the Fc region found in a native antibody. Native sequence human Fc regions include a native sequence human IgG1 Fc region, native sequence human IgG2 Fc region, native sequence human IgG3 Fc region, and native sequence human IgG4 Fc region, as well as naturally occurring variants thereof. Native sequence Fc includes the various allotypes of Fcs (see, e.g., Jefferis et al., mAbs, 1(4): 332-338 (2009)).

In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/I332E), GASDALIE (G236A/S239D/A330L/I332E), V9 (G237D/P238D/P271G/A330R), and V11 (G237D/P238D/H268D/P271G/A330R), and/or one or more mutations at the following amino acids: E345R, E233, G237, P238, H268, P271, L328 and A330. Additional Fc region modifications for modulating Fc receptor binding are described in, for example, U.S. Patent Application Publication 2016/0145350 and U.S. Pat. Nos. 7,416,726 and 5,624,821, which are hereby incorporated by reference in their entireties herein.

In some embodiments, the Fc region of the binding agents are modified to have an altered glycosylation pattern of the Fc region compared to the native non-modified Fc region.

Human immunoglobulin is glycosylated at the Asn297 residue in the Cγ2 domain of each heavy chain. This N-linked oligosaccharide is composed of a core heptasaccharide, N-acetylglucosamine4Mannose3 (GlcNAc4Man3). Removal of the heptasaccharide with endoglycosidase or PNGase F is known to lead to conformational changes in the Fc region, which can significantly reduce binding affinity to activating FcγR and lead to decreased effector function. The core heptasaccharide is often decorated with galactose, bisecting GlcNAc, fucose, or sialic acid, which differentially impacts Fc binding to activating and inhibitory FcγR. Additionally, it has been demonstrated that a2,6-sialyation enhances anti-inflammatory activity in vivo, while defucosylation leads to improved FcγRIIIa binding and a 10-fold increase in antibody-dependent cellular cytotoxicity and antibody-dependent phagocytosis. Specific glycosylation patterns, therefore, can be used to control inflammatory effector functions.

In some embodiments, the modification to alter the glycosylation pattern is a mutation. For example, a substitution at Asn297. In some embodiments, Asn297 is mutated to glutamine (N297Q). Methods for controlling immune response with antibodies that modulate FcγR-regulated signaling are described, for example, in U.S. Pat. No. 7,416,726 and U.S. Patent Application Publications 2007/0014795 and 2008/0286819, which are hereby incorporated by reference in their entireties.

In some embodiments, the binding agents are modified to contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function. In some embodiments, the binding agents are engineered to be afucosylated.

In some embodiments, the entire Fc region is exchanged with a different Fc region, so that the Fab region is conjugated to a non-native Fc region. For example, the Fab region of atezolizumab, which normally comprises an IgG1 Fc region, can be conjugated to IgG2, IgG3, IgG4, or IgA, or the Fab region of nivolumab, which normally comprises an IgG4 Fc region, can be conjugated to IgG1, IgG2, IgG3, IgA1, or IgG2. In some embodiments, the Fc modified binding agent with a non-native Fc domain also comprises one or more additional amino acid modification, such as the S228P mutation within the IgG4 Fc, that modulate the stability of the Fc domain described. In some embodiments, the Fc modified binding agent with a non-native Fc domain also comprises one or more amino acid modifications described herein that modulate Fc binding to FcR.

In some embodiments, the modifications that modulate the binding of the Fc region to FcR do not alter the binding of the Fab region to its antigen when compared to the non-modified Fab. In other embodiments, the modifications that modulate the binding of the Fc region to FcR also increase the binding of the Fab region to its antigen when compared to the non-modified Fab.

In some embodiments, the Fc region is modified by attachment or inclusion of a transforming growth factor beta 1 (TGFβ1) receptor, or a fragment thereof, that is capable of binding TGFβ1. For example, the receptor can be TGFβ receptor II (TGFβRII) (see U.S. Pat. No. 9,676,863, incorporated herein in its entirety). In some embodiments, the TGFβ receptor is a human TGFβ receptor. In some embodiments, the Fc region (e.g., IgG) has a C-terminal fusion to a TGFβ receptor (e.g., TGFβRII) extracellular domain (ECD; e.g., amino acids 24-159 of SEQ ID NO: 9 of U.S. Pat. No. 9,676,863). An “Fc linker” may be used to attach the IgG to the TGFβR extracellular domain, for example, a G₄S₄G Fc linker. The Fc linker may be a short, flexible peptide that allows for the proper three-dimensional folding of the molecule while maintaining the binding-specificity to the targets. In some embodiments, the N-terminus of the TGFβ receptor is fused to the Fc region (with or without an Fc linker). In some embodiments, the C-terminus of the immunoglobulin heavy chain is fused to the TGFβ receptor (with or without an Fc linker). In some embodiments, the C-terminal lysine residue of the antibody heavy chain is mutated to alanine. In some embodiments, the antibody includes SEQ ID NO: 166.

The PD-L1 binding agent can have any suitable affinity to a PD-L1 protein or an epitope thereof. The term “affinity” refers to the equilibrium constant for the reversible binding of two agents and is expressed as the dissociation constant (K_{D}). Affinity of a binding agent to a ligand, such as affinity of an antibody for an epitope, can be, for example, from about 1 picomolar (pM) to about 100 micromolar (μM) (e.g., from about 1 picomolar (pM) to about 1 nanomolar (nM), from about 1 nM to about 1 micromolar (μM), or from about 1 μM to about 100 μM). In one embodiment, the PD-L1-binding agent can bind to an PD-L1 protein with a K_{D} less than or equal to 1 nanomolar (e.g., 0.9 nM, 0.8 nM, 0.7 nM, 0.6 nM, 0.5 nM, 0.4 nM, 0.3 nM, 0.2 nM, 0.1 nM, 0.05 nM, 0.025 nM, 0.01 nM, 0.001 nM, or a range defined by any two of the foregoing values). In another embodiment, the PD-L1-binding agent can bind to PD-L1 with a K_{D} less than or equal to 200 pM (e.g., 190 pM, 175 pM, 150 pM, 125 pM, 110 pM, 100 pM, 90 pM, 80 pM, 75 pM, 60 pM, 50 pM, 40 pM, 30 pM, 25 pM, 20 pM, 15 pM, 10 pM, 5 pM, 1 pM, or a range defined by any two of the foregoing values). Immunoglobulin affinity for an antigen or epitope of interest can be measured using any art-recognized assay. Such methods include, for example, fluorescence activated cell sorting (FACS), separable beads (e.g., magnetic beads), surface plasmon resonance (SPR), solution phase competition (KINEXA™), antigen panning, and/or ELISA (see, Janeway et al. (eds.), Immunobiology, 9th Ed., Garland Publishing, New York, N.Y. (2017)).

### Nucleic Acids

The invention also provides nucleic acids that encode the immunoglobulin heavy chain polypeptide and/or the immunoglobulin light chain polypeptide of the PD-L1-binding agent.

The term “nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

The nucleic acid can be part of a vector. Thus, also provided is a vector comprising one or more nucleic acid sequences encoding the immunoglobulin heavy chain polypeptide, the immunoglobulin light chain polypeptide, or both, of the PD-L1 binding agent. Any type of vector can be used, particularly an expression vector useful for expressing the polypeptides in a cell. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994)).

The vector preferably comprises expression control sequences, such as promoters, enhancers, polyadenylation signals, transcription terminators, internal ribosome entry sites (IRES), and the like, that provide for the expression of the coding sequence in a host cell. Exemplary expression control sequences are known in the art and described in, for example, Goeddel, Gene Expression Technology: Methods in Enzymology, Vol. 185, Academic Press, San Diego, Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the American Type Culture Collection (ATCC) as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, Calif.), LACSWITCH™ system (Stratagene, San Diego, Calif.), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol., 308: 123-144 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences.

The vector also can comprise a “selectable marker gene.” The term “selectable marker gene,” as used herein, refers to a nucleic acid sequence that allow cells expressing the nucleic acid sequence to be specifically selected for or against, in the presence of a corresponding selective agent. Suitable selectable marker genes are known in the art and described in, e.g., International Patent Application Publications WO 1992/008796 and WO 1994/028143; Wigler et al., Proc. Natl. Acad. Sci. USA, 77: 3567-3570 (1980); O'Hare et al., Proc. Natl. Acad. Sci. USA, 78: 1527-1531 (1981); Mulligan & Berg, Proc. Natl. Acad. Sci. USA, 78: 2072-2076 (1981); Colberre-Garapin et al., J. Mol. Biol., 150: 1-14 (1981); Santerre et al., Gene, 30: 147-156 (1984); Kent et al., Science, 237: 901-903 (1987); Wigler et al., Cell, 11: 223-232 (1977); Szybalska & Szybalski, Proc. Natl. Acad. Sci. USA, 48: 2026-2034 (1962); Lowy et al., Cell, 22: 817-823 (1980); and U.S. Pat. Nos. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.) and pBK-CMV from Stratagene (La Jolla, Calif.) represent non-limiting examples of an episomal vector that uses T-antigen and the SV40 origin of replication in lieu of EBNA1 and oriP.

Other suitable vectors include integrating expression vectors, which may randomly integrate into the host cell's DNA, or may include a recombination site to enable the specific recombination between the expression vector and the host cell's chromosome. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, Calif.) (e.g., pcDNA™5/FRT), or the cre-lox system, such as can be found in the pExchange-6 Core Vectors from Stratagene (La Jolla, Calif.). Examples of vectors that randomly integrate into host cell chromosomes include, for example, pcDNA3.1 (when introduced in the absence of T-antigen) from Invitrogen (Carlsbad, Calif.), UCOE from Millipore (Billerica, Mass.), and pCI or pFN10A (ACT) FLEXI™ from Promega (Madison, Wis.).

Viral vectors also can be used. Representative commercially available viral expression vectors include, but are not limited to, the adenovirus-based Per.C6 system available from Crucell, Inc. (Leiden, The Netherlands), the lentiviral-based pLP1 from Invitrogen (Carlsbad, Calif.), and the retroviral vectors pFB-ERV plus pCFB-EGSH from Stratagene (La Jolla, Calif.).

### Cells

Nucleic acid sequences encoding the heavy and light chain immunoglobulin sequences can be provided to a cell on the same vector (i.e., in cis). A unidirectional promoter can be used to control expression of each nucleic acid sequence. In another embodiment, a combination of bidirectional and unidirectional promoters can be used to control expression of multiple nucleic acid sequences. Nucleic acid sequences encoding the inventive amino acid sequences alternatively can be provided to the population of cells on separate vectors (i.e., in trans). Each of the nucleic acid sequences in each of the separate vectors can comprise the same or different expression control sequences. The separate vectors can be provided to cells simultaneously or sequentially.

The vector(s) comprising the nucleic acid(s) encoding the inventive amino acid sequences can be introduced into a host cell that is capable of expressing the polypeptides encoded thereby, including any suitable prokaryotic or eukaryotic cell. As such, the invention provides an in vitro (isolated) cell or cell line comprising the inventive vector, which expresses the immunoglobulin heavy and light chain polypeptides. Preferred host cells are those that can be easily and reliably grown, have reasonably fast growth rates, have well characterized expression systems, and can be transformed or transfected easily and efficiently.

Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DH5α, DH10, MC1061 (ATCC No. 53338), and CC102).

Preferably, the vector is introduced into a eukaryotic cell. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Preferred yeast cells include, for example, Saccharomyces cerevisiae and Pichia pastoris.

Suitable insect cells are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Preferred insect cells include Sf-9 and HI5 (Invitrogen, Carlsbad, Calif.).

Preferably, mammalian cells are utilized in the invention. A number of suitable mammalian host cells are known in the art, and many are available from ATCC. Examples of suitable mammalian cells include, but are not limited to, Chinese hamster ovary cells (CHO) cells, such as CHO-K1 cells (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), JIMT-1, and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, mouse L-929 cells, and BHK or HaK hamster cell lines, all of which are available from the ATCC. Methods for selecting suitable mammalian host cells and methods for transformation, culture, amplification, screening, and purification of cells are known in the art.

The mammalian cell can be a human cell. For example, the mammalian cell can be a human lymphoid or lymphoid derived cell line, such as a cell line of pre-B lymphocyte origin. Examples of human lymphoid cells lines include, without limitation, RAMOS (CRL-1596), Daudi (CCL-213), EB-3 (CCL-85), DT40 (CRL-2111), 18-81 (Jack et al., Proc. Natl. Acad. Sci. USA, 85: 1581-1585 (1988)), Raji cells (CCL-86), and derivatives thereof.

A nucleic acid sequence encoding the inventive amino acid sequence may be introduced into a cell by “transfection,” “transformation,” or “transduction.” “Transfection,” “transformation,” or “transduction,” as used herein, refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

### Compositions

The invention provides a composition comprising the PD-L1-binding agent or nucleic acid(s) encoding same optionally in a vector. Preferably, the composition is a pharmaceutically acceptable (e.g., physiologically acceptable) composition, which comprises a carrier, preferably a pharmaceutically acceptable (e.g., physiologically acceptable) carrier, and the PD-L1 binding agent or nucleic acid(s) encoding same. Any suitable carrier can be used within the context of the invention, and such carriers are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, Pa. (2001).

The composition can be formulated for parenteral administration, such as IV administration or administration into a body cavity or lumen of an organ. Alternatively, the composition can be injected intra-tumorally. Compositions for injection will commonly comprise the active ingredient dissolved or suspended in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These compositions desirably are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The composition can contain any suitable concentration of the PD-L1-binding agent or nucleic acid(s) encoding same optionally in a vector, in some embodiments, a concentration effective to elicit a therapeutic response. The concentration can vary widely, and will be selected primarily based on fluid volumes, viscosities, body weight, and the like, in accordance with the particular mode of administration selected and the patient's needs. In certain embodiments, the concentration of the inventive immunoglobulin heavy chain polypeptide, the inventive immunoglobulin light chain polypeptide, the inventive PD-L1-binding agent, the inventive nucleic acid sequence encoding any of the foregoing, or the inventive vector comprising the inventive nucleic acid sequence in a solution formulation for injection will range from about 0.1% (w/w) to about 10% (w/w).

### Methods

The PD-L1 binding agents provided herein can be used for any suitable purpose. For instance, the PD-L1 binding agents can target PD-L1 expressing cells, and be used to deliver a payload to such cells. The payload can be any agent that is desired to be delivered to the cell (e.g., therapeutic agent, diagnostic/imaging agent, etc.). According, one aspect of the disclosure provides a method of delivering a payload to a cell expressing PD-L1, the method comprising administering to the cell, or mammal comprising the cell, a PD-L1 binding agent provided herein conjugated to the payload.

Provided herein is a method of enhancing an immune response in a mammal comprising administering a PD-L1 binding agent alone or conjugated to an appropriate drug or other agent suitable for increasing an immune response, or composition comprising same, as described herein to the mammal. The immune response can be an immune response to any type of antigen for use in any application in which it is desirable to enhance an immune response to the antigen. In some embodiments, the antigen can be an infectious agent or pathogen (e.g., a virus or bacteria) or a cancer antigen (e.g., an endogenous cancer antigen in the cell and peptide vaccines or a neoantigen).

In another embodiment, there is provided a method of reducing (e.g., inhibiting) an immune response in a mammal comprising administering a PD-L1 binding agent alone or conjugated to an appropriate drug or other agent suitable for reducing an immune response, or composition comprising same, as described herein to the mammal. The immune response can be, for instance, an autoimmune response.

In this respect, the invention also provides a method of treating a disease, condition, or disorder responsive to PD-L1 inhibition in a mammal by administering a PD-L1 binding agent, or composition comprising same, as described herein to the mammal. In some embodiments, the disease or disorder is characterized by the improper expression (e.g., overexpression) or increased activity of a PD-L1 protein, which causes or contributes to the pathological effects of the disease, such that a decrease in PD-L1 protein levels or activity has a therapeutic benefit in mammals, preferably humans.

As used herein, the terms “treat,” “treatment,” and “treating” refer to any indicia of success in the treatment or amelioration of an injury, pathology, condition (e.g., cancer, infection, or autoimmune disorder), or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

As used herein, the term “cancer” includes any form of cancer, including but not limited to, solid tumor cancers (e.g., skin, lung, prostate, breast, gastric, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors.

Any PD-L1 expressing or overexpressing cancer is a suitable cancer to be treated by the subject methods and compositions. As used herein “PD-L1 expression” refers to a cell that has a PD-L1 receptor on the cell's surface. As used herein “PD-L1 overexpression” refers to a cell that has more PD-L1 receptors as compared to corresponding non-cancer cell of the same type.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, stomach, gastroesophageal junction, and colon); adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma (e.g., head and neck squamous cell carcinoma); transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, and skin.

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Merkel cell carcinoma is a rare type of skin cancer that usually appears as a flesh-colored or bluish-red nodule. It frequently appears on the face, head or neck. Merkel cell carcinoma is also called neuroendocrine carcinoma of the skin. In some embodiments, the Merkel cell carcinoma has metastasized when administration occurs.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CIVIL), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Sternberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, and invasion of surrounding or distant tissues or organs, such as lymph nodes.

As used herein, the phrases “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs, therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

As used herein, the phrase “autoimmune disorder” refers to means a disease or disorder caused by a reaction produced by the body against its own tissues or organs or cosegregation or manifestation of these disorders or condition associated with them. Examples of autoimmune diseases or disorders include, but are not limited to, arthritis (rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile rheumatoid arthritis, osteoarthritis, a progressive chronic arthritis, deforming arthritis, primary chronic arthritis, reactive arthritis and ankylosing spondylitis), inflammatory hyperproliferative skin diseases, psoriasis such as plaque psoriasis, guttate psoriasis, pustular psoriasis and psoriasis of nails; dermatitis including contact dermatitis, chronic contact dermatitis, allergic dermatitis, allergic contact dermatitis, dermatitis herpetiformis, and atopic dermatitis; coupled with X-linked hyper-IgM-syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as MS, is accompanied by breach of the spinal cord and visual organs, primary progressive MS (APP) to and relapsing-remitting MS (RRMS), progressive systemic sclerosis, atherosclerosis, arteriosclerosis, multiple sclerosis, and ataxic sclerosis; inflammatory bowel disease (IBD) (e.g., Crohn's disease, autoimmune gastrointestinal disorders, colitis such as ulcerative colitis, ulcerative colitis, microscopic colitis, collagenous colitis, polypoid colitis, nekroziruyuschy enterocolitis, and transmural colitis, and autoimmune inflammatory bowel disease); pyoderma gangrenosum, erythema nodosum, primary sclerosing cholangitis, episcleritis), respiratory distress syndrome, including adult respiratory distress syndrome (ARDS) or acute respiratory distress syndrome, meningitis, inflammation of all or part of the uveal tract, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and encephalitis with lesions limbs and/or the brain stem, uveitis, such as anterior uveitis, acute anterior uveitis, granulomatous uveitis, negranulomatozny uveitis fakoantigenny uveitis, posterior uveitis, or autoimmune uveitis, glomerulonephritis (GN) with nephrotic syndrome or without nephrotic syndrome such as chronic or acute glomerulonephritis such as primary GN, immune-mediated GN, membranous GN (membranous nephropathy), idiopathic membranous GN or idiopathic membranous nephropathy, membranous proliferative or membranoproliferative ativnost GN (MPGN), including glumerulonefrit type I and type II, and rapidly progressive GN, allergic conditions, allergic reaction, eczema including allergic or atopic eczema; asthma, such as bronchial asthma and autoimmune asthma; conditions caused by infiltration of T cells and chronic inflammatory responses; chronic inflammatory lung disease, autoimmune myocarditis, leukocyte adhesion is insufficient; systemic lupus erythematosus (systemic lupus erythematodes) (SLE) such as cutaneous SLE or subacute cutaneous SLE, lupus neonatal syndrome (HRV), disseminated lupus erythematosus, lupus (including nephritis, cerebritis, child lupus, nonrenal lupus, extrarenal lupus, discoid lupus, alopecia); juvenile diabetes (type I), including child insulin dependent diabetes mellitus (IDDM), adult diabetes mellitus (diabetes type II), autoimmune diabetes, idiopathic diabetes insipidus, immune responses associated with acute hypersensitivity and delayed type hypersensitivity mediated by cytokines and T lymphocytes; tuberculosis, sarcoidosis, granulomatosis including lymphomatoid granulomatosis, Wegener's granulomatosis, agranulocytosis; vasculitides including vasculitis (including vasculitis, large blood vessels (including polymyalgia rheumatica and giant cell arteritis (Takayasu's)), vasculitis secondary blood vessels (including Kavazaki disease and polyarteritis nodosa), polyarteritis capillary vasculitis CNS nekroziruyuschy vasculitis, cutaneous vasculitis or vasculitis associated hypersensitivity nekroziruyuschy systemic vasculitis and ANCA-associated vasculitis, such as vasculitis or Cherga-Strauss syndrome (SCHSH), temporal arteritis, aplastic anemia, auto mmunnaya aplastic anemia, a positive anemia, Coombs, anemia, Diamond-Blekfana, hemolytic anemia or immune hemolytic anemia including autoimmune hemolytic anemia (AIHA), pernicious anemia (pernicious anemia), Addison's disease, pure red cell anemia or aplasia (PRCA), Factor VIII deficiency, hemophilia A, autoimmune neutropenia, pancytopenia, leukopenia, diseases that lead to leukocyte diapedesis, CNS inflammatory disorder, multiple organ injury syndrome, such as a secondary syndrome, ass of dissociated with sepsis, trauma or hemorrhage; diseases mediated by complex formation “antigen-antibody” disease glomerular basement membranes, catalysed reaction of the antibody-antigen, antiphospholipid syndrome, allergic neurite disease/Behcet's syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgar, leaf-pemphigus, pemphigoid, mucous membrane pemphigoid membranous and pemphigus erythematosus), utoimmunnaya poliendokrinopatiya disease or Reiter's syndrome, nephritis associated with immune complexes, antibody-mediated nephritis, neuromyelitis optica, polyneuropathies, chronic neuropathy such as IgM-polyneuropathy or IgM-mediated neuropathy, thrombocytopenia (e.g., developing in a patient with myocardial infarction), including thrombotic thrombocytopenic purpura (TTP) and autoimmune or immune-mediated thrombocytopenia such as idiopathic thrombocytopenic purpura (ITP) including chromel matic or acute ITP; autoimmune disease testis and ovary including autoimmune orchitis and oophoritis, primary hypothyroid, gipoparatireoidit, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroid, Grave's disease, polyglandular syndromes such as autoimmune polyglandular syndromes (or polyglandular endocrinopathy syndromes), paraneoplastic syndromes, including neurologic paraneoplastic syndromes such as myasthenic syndrome, Lambert-Eaton syndrome or Eaton-Lambert syndrome “stiff-man,” encephalomyelitis such as allergic encephalomyelitis (or encephalomyelitis allergica) and experimental allergic encephalomyelitis (EAE), myasthenia gravis such as myasthenia gravis associated with thymoma, cerebellar degeneration, nevromiotoniya, opsoclonus or syndrome (LNG) and senses neuropathy, multifocal motor neuropathy, the system We, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transmissible, unlike NSIP); Guillain-Barre syndrome, Berger's disease (IgA-nephropathy), idiopathic IgA-nephropathy, linear IgA-dermatosis, primary biliary cirrhosis, pnevmonotsirroz, autoimmune enteropathy syndrome, bowel disease or celiac disease, intestinal sprue (gluten enteropathy), not amenable sprue treatment, idiopathic sprue, cryoglobulinemia, amilotrofichesky lateral sclerosis (ALS; Louis Gehrig's disease), coronary heart disease; autoimmune ear disease such as autoimmune inner ear disease (AZVU); autoimmune hearing loss; “Dancing eye” syndrome (LNG), polychondritis such as refractory, or relapsing polychondritis; pulmonary alveolar proteinosis, amyloidosis, scleritis, non-cancerous lymphocytosis, a primary lymphocytosis, including monoclonal B cell lymphocytosis (e.g., benign monoclonal gammopathy and monoclonal gammopathy of unknown etiology, MGUS); peripheral neuropathy, paraneoplastic syndrome; “Channelopathy,” such as epilepsy, migraine, arrhythmia, muscular disorders, deafness, blindness, periodic paralysis and “channelopathy” CNS, autism, inflammatory myopathy, focal and segmental glomerulosclerosis (OSGS), endocrine ophthalmopathy, uveoretinitis, chorioretinitis, autoimmune liver disease, fibromyalgia, multiple endocrine failure, Schmidt's syndrome, adrenalit, gastric atrophy, presenile dementia, demyelinating diseases such as autoimmune demyelinating diseases, diabetic nefropa ment, Dressler's syndrome, alopecia areata, CREST syndrome (calcinosis, Raynau's phenomenon, impaired esophageal motility, sclerodactyly, and telangiectasia), autoimmune infertility in both men and women, mixed connective tissue disease, Chagas' disease, rheumatic fever, recurrent miscarriage, lung disease farmers, erythema multiforme, cardiotomy syndrome, Cushing's syndrome, pulmonary disease, bird lovers, allergic granulomatous vasculitis, benign lymphocytic angiitis, Alport syndrome, alveolitis, such as allergies cal alveolitis and fibrosing alveolitis, interstitial lung disease, transfusion disease, leprosy, malaria, leishmaniasis, kipanosomoz, schistosomiasis, askarioz, aspergillosis, Semptera syndrome, Caplan's syndrome, dengue, endocarditis, endomyocardial fibrosis, diffuse interstitial pulmonary fibrosis, interstitial pulmonary fibrosis, idiopathic pulmonary fibrosis, cystic fibrosis, endophthalmitis, erythema elevated resistant, fetal erythroblastosis, eosinophilic fasciitis, Shulman's syndrome, Felty's syndrome, flyarioz, cyclitis such as cartilage onichesky cycles geterohronichesky cycles or cycles iridotsyklit Fuchs, Henoch-Schonlein purpura, infection caused by the human immunodeficiency virus (HIV) infections caused by echovirus; cardiomyopathy, Alzheimer's disease, infections caused by parvovirus; infections caused by rubella virus; syndrome that develops after vaccination; hereditary infection caused by rubella virus; infections caused by Epstein-Barr virus; Mumps, Evans syndrome, autoimmune gonadal failure, Sydenham's chorea, post-streptococcal nephritis, thromboangiitis obliterans, thyrotoxicosis, tabes, choroiditis, giant cell polymyalgia, endocrine ophthalmopathy, pneumonitis associated with chronic hypersensitivity, keratoconjunctivitis sicca, epidemic keratoconjunctivitis, idiopathic nephritis syndrome, nephropathy, characterized by minimal changes of renal tissue, benign hereditary caused by ischemia and rap rfuzionnye lesions, autoimmune disease of the retina, inflammation of the joints, bronchitis, chronic obstructive airway disease, silicosis, aphthae, aphthous stomatitis, arteriosclerotic disorders, aspermiogenez, autoimmune hemolysis, Beck disease, cryoglobulinemia, Dupuytren's contracture, fakoanafilakticheskaya endophthalmitis, allergic enteritis, nodose lepromatous erythema, idiopathic facial paralysis, chronic fatigue syndrome, rheumatic fever, Hamman Rich syndrome; sensorineural hearing loss, paroxysmal hemoglobinuria, hypogonadism, regional ileitis, leukopenia, infectious mononucleosis, transverse myelitis, primary idiopathic miksidema, nephrosis, sympathetic ophthalmia, granulomatous orchitis, pancreatitis, acute polyradiculitis, pyoderma gangrenosum, thyroiditis Quervain acquired atrophy of the spinal cord, infertility, caused by anti-sperm, non-malignant thymoma, vitiligo, severe combined immunodeficiency (SCID) and diseases caused by a virus Epstein and Barra; acquired immunodeficiency syndrome (AIDS), parasitic diseases such as leishmaniasis, toxic shock syndrome, food poisoning, conditions caused by infiltration of T-cells, the lack of leukocyte adhesion, immune responses associated with acute hypersensitivity and delayed type hypersensitivity mediated by cytokines and T-lymphocytes, diseases associated with leukocyte diapedesis, multiple organ injury syndrome, a disease mediated by the formation of the complex antigen-antibody Disease glomerular basement membranes, allergic neuritis, autoimmune poliendokrinopatiya, oophoritis, primary myxedema, autoimmune atrophic gastritis, sympathetic ophthalmia, rheumatic diseases, mixed connective tissue disease, nephrotic syndrome, insulitis, polyendocrine failure, neuropathy of peripheral organs, autoimmune polyglandular syndrome type I, idiopathic gipoparatireoidit adults (IIV), total alopecia, congestive cardiomyopathy, acquired epidermolysis bullosa (PBE), hemochromatosis, myocarditis, nephrotic syndrome, primary sclerosing cholangitis, purulent or non-purulent sinusitis, acute or chronic sinusitis; grid sinusitis, frontal sinusitis, maxillary sinusitis or sphenoiditis; eozonofilnye disorder such as eosinophilia, pulmonary infiltrating eosinophilia, eosinophilia-myalgia syndrome, Leffler syndrome, chronic eosinophilic pneumonia, tropical pulmonary eosinophilia, allergic pulmonary aspergillosis, aspergilloma, or granulomas containing eosinophils; anaphylaxis, seronegative spondylitis, multiple endocrine autoimmune disease, sclerosing cholangitis, scleritis, episcleritis, chronic mucocutaneous candidiasis, Bruton's syndrome, transient hypogammaglobulinemia in children with Wiskott-Aldrich syndrome, ataxia-telangiectasia syndrome, autoimmune disorders associated with collagen disease, rheumatism, neurological disease, ischemic reperfusion injury, reaction to a drop in blood pressure, vascular dysfunction, angiodysplasia, tissue damage, vascular ischemia, hyperalgesia, cerebral ischemia, and disease accompanied by vascularization, allergic disorders associated with hypersensitivity, glomerulonephritis, reperfusion injury, reperfusion injury of myocardial or other tissues, dermatoses with the components of acute inflammation, acute purulent meningitis or other inflammatory disorders of the central nervous system, inflammatory diseases of the eye and orbit; syndromes associated with granulocyte transfusion; toxicity induced by cytokines, acute serous inflammation, chronic intractable inflammation, pyelitis, diabetic retinopathy, diabetic lesion of large arteries, peripheral arterial hyperplasia, peptic ulcer, and endometriosis.

As used herein, the phrase “infection” refers to a condition caused by an infectious pathogen, e.g., virus, bacteria, fungus, or parasite.

As used herein the phrases “effective amount” and “therapeutically effective amount” refer to a dose of a substance such as a binding agent that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)).

As used herein, the terms “recipient,” “individual,” “subject,” “host,” and “patient” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The inventive method also can be used to treat any type of infectious disease (i.e., a disease or disorder caused by a bacterium, a virus, a fungus, or a parasite). Examples of infectious diseases that can be treated by the inventive method include, but are not limited to, diseases caused by a human immunodeficiency virus (HIV), a respiratory syncytial virus (RSV), an influenza virus, a dengue virus, a hepatitis B virus (HBV, or a hepatitis C virus (HCV)). Administration of a composition comprising the inventive immunoglobulin heavy chain polypeptide, the inventive immunoglobulin light chain polypeptide, the inventive PD-L1-binding agent, the inventive nucleic acid sequence encoding any of the foregoing, or the inventive vector comprising the inventive nucleic acid sequence induces an immune response against a cancer or infectious disease in a mammal. An “immune response” can entail, for example, antibody production and/or the activation of immune effector cells (e.g., T-cells).

The inventive methods comprise administering a “therapeutically effective amount” of the binding agent. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the binding agent to elicit a desired response in the individual. For example, a therapeutically effective amount of a binding agent of the invention is an amount which decreases PD-L1 protein bioactivity in a human and/or enhances the immune response against a cancer or infectious disease.

Alternatively, the pharmacologic and/or physiologic effect may be prophylactic, i.e., the effect completely or partially prevents a disease or symptom thereof. In this respect, the inventive method comprises administering a “prophylactically effective amount” of the binding agent. A “prophylactically effective amount” refers to an amount effective, at dosages and for periods of time necessary, to achieve a desired prophylactic result (e.g., prevention of disease onset).

A typical dose can be, for example, in the range of 1 μg/kg to 20 mg/kg of animal or human body weight; however, doses below or above this exemplary range are within the scope of the invention. The daily parenteral dose can be about 0.00001 μg/kg to about 20 mg/kg of total body weight (e.g., about 0.001 μg/kg, about 0.1 μg/kg, about 1 μg/kg, about 5 μg/kg, about 10 μg/kg, about 100 μg/kg, about 500 μg/kg, about 1 mg/kg, about 5 mg/kg, about 10 mg/kg, or a range defined by any two of the foregoing values), preferably from about 0.1 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), more preferably from about 1 μg/kg to 5 mg/kg of total body weight (e.g., about 3 μg/kg, about 15 μg/kg, about 75 μg/kg, about 300 μg/kg, about 900 μg/kg, about 2 mg/kg, about 4 mg/kg, or a range defined by any two of the foregoing values), and even more preferably from about 0.5 to 15 mg/kg body weight per day (e.g., about 1 mg/kg, about 2.5 mg/kg, about 3 mg/kg, about 6 mg/kg, about 9 mg/kg, about 11 mg/kg, about 13 mg/kg, or a range defined by any two of the foregoing values). Therapeutic or prophylactic efficacy can be monitored by periodic assessment of treated patients. For repeated administrations over several days or longer, depending on the condition, the treatment can be repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

The composition comprising an effective amount of the inventive immunoglobulin heavy chain polypeptide, the inventive immunoglobulin light chain polypeptide, the inventive binding agent, the inventive nucleic acid sequence encoding any of the foregoing, or the inventive vector comprising the inventive nucleic acid sequence can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection.

Once administered to a mammal (e.g., a human), the biological activity of the inventive binding agent can be measured by any suitable method known in the art. For example, the biological activity can be assessed by determining the stability of a particular binding agent. In one embodiment of the invention, the binding agent (e.g., an antibody) has an in vivo half life between about 30 minutes and 45 days (e.g., about 30 minutes, about 45 minutes, about 1 hour, about 2 hours, about 4 hours, about 6 hours, about 10 hours, about 12 hours, about 1 day, about 5 days, about 10 days, about 15 days, about 25 days, about 35 days, about 40 days, about 45 days, or a range defined by any two of the foregoing values). In another embodiment, the PD-L1-binding agent has an in vivo half life between about 2 hours and 20 days (e.g., about 5 hours, about 10 hours, about 15 hours, about 20 hours, about 2 days, about 3 days, about 7 days, about 12 days, about 14 days, about 17 days, about 19 days, or a range defined by any two of the foregoing values). In another embodiment, the binding agent has an in vivo half life between about 10 days and about 40 days (e.g., about 10 days, about 13 days, about 16 days, about 18 days, about 20 days, about 23 days, about 26 days, about 29 days, about 30 days, about 33 days, about 37 days, about 38 days, about 39 days, about 40 days, or a range defined by any two of the foregoing values).

The PD-L1 binding agent of the invention may be administered alone or in combination with other drugs, either as separate moieties or as a conjugate with the PD-L1 binding agent. For example, the binding agent can be administered in combination with other agents for the treatment or prevention of the diseases disclosed herein. In this respect, the binding agent can be used in combination with at least one other anticancer agent including, for example, any chemotherapeutic agent known in the art, ionization radiation, small molecule anticancer agents, cancer vaccines, biological therapies (e.g., other monoclonal antibodies, cancer-killing viruses, gene therapy, and adoptive T-cell transfer, immune conjugates or cytotoxic antibody drug conjugates), and/or surgery. When the inventive method treats an infectious disease, the binding agent can be administered in combination with at least one anti-bacterial agent or at least one anti-viral agent. In this respect, the anti-bacterial agent can be any suitable antibiotic known in the art. The anti-viral agent can be any vaccine of any suitable type that specifically targets a particular virus (e.g., live-attenuated vaccines, subunit vaccines, recombinant vector vaccines, and small molecule anti-viral therapies (e.g., viral replication inhibitors and nucleoside analogs).

In another embodiment, the inventive binding agent can be administered in combination with other agents that inhibit innate and adaptive immune checkpoint pathways. For example, the inventive binding agent can be administered in combination with agents that inhibit or antagonize the CTLA-4, TIM-3, LAG-3, CD47, CD24, and SIRPA pathways.

In addition to therapeutic uses, the binding agent described herein can be used in diagnostic or research applications. In this respect, the binding agent can be used in a method to diagnose a cancer or infectious disease. In a similar manner, the binding agent can be used in an assay to monitor PD-L1 protein levels in a subject being tested for a disease or disorder that is associated with abnormal PD-L1 expression. Research applications include, for example, methods that utilize the binding agent and a label to detect a PD-L1 protein in a sample, e.g., in a human body fluid or in a cell or tissue extract. The binding agent can be used with or without modification, such as covalent or non-covalent labeling with a detectable moiety. For example, the detectable moiety can be a radioisotope (e.g., ³H, ¹⁴C, ³²P, ³⁵S, or ¹²⁵I), a fluorescent or chemiluminescent compound (e.g., fluorescein isothiocyanate, rhodamine, or luciferin), an enzyme (e.g., alkaline phosphatase, beta-galactosidase, or horseradish peroxidase), or prosthetic groups. Any method known in the art for separately conjugating an antigen-binding agent (e.g., an antibody) to a detectable moiety may be employed in the context of the invention (see, e.g., Hunter et al., Nature, 194: 495-496 (1962); David et al., Biochemistry, 13: 1014-1021 (1974); Pain et al., J. Immunol. Meth., 40: 219-230 (1981); and Nygren, J. Histochem. and Cytochem., 30: 407-412 (1982)).

PD-L1 protein levels can be measured using the inventive binding agent by any suitable method known in the art. Such methods include, for example, immunohistochemistry, immunofluorescence, radioimmunoassay (RIA), and FACS. Normal or standard expression values of PD-L1 protein can be established using any suitable technique, e.g., by combining a sample comprising, or suspected of comprising, a PD-L1 polypeptide with a PD-L1-specific antibody under conditions suitable to form an antigen-antibody complex. The antibody is directly or indirectly labeled with a detectable substance to facilitate detection of the bound or unbound antibody. Suitable detectable substances include various enzymes, prosthetic groups, fluorescent materials, luminescent materials, and radioactive materials (see, e.g., Zola, Monoclonal Antibodies: A Manual of Techniques, CRC Press, Inc. (1987)). The amount of PD-L1 polypeptide expressed in a sample is then compared with a standard value.

### Kits

The binding agent can be provided in a kit, i.e., a packaged combination of reagents in predetermined amounts with instructions for performing a diagnostic assay. If the binding agent is labeled with an enzyme, the kit desirably includes substrates and cofactors required by the enzyme (e.g., a substrate precursor which provides a detectable chromophore or fluorophore). In addition, other additives may be included in the kit, such as stabilizers, buffers (e.g., a blocking buffer or lysis buffer), and the like. The relative amounts of the various reagents can be varied to provide for concentrations in solution of the reagents which substantially optimize the sensitivity of the assay. The reagents may be provided as dry powders (typically lyophilized), including excipients which on dissolution will provide a reagent solution having the appropriate concentration.

### Examples of Non-Limiting Aspects of the Disclosure

Aspects, including embodiments, of the invention described herein may be beneficial alone or in combination, with one or more other aspects or embodiments. Without limiting the foregoing description, certain non-limiting aspects of the disclosure numbered (1)-(32) are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A programmed death-ligand 1 (PD-L1) binding agent comprising an immunoglobulin heavy chain variable region polypeptide and an immunoglobulin light chain variable region polypeptide, wherein:

the immunoglobulin heavy chain variable region polypeptide comprises a complementarity determining region 1 (HCDR1) comprising any one of SEQ ID NOs: 1-14 or 167, a complementarity determining region 2 (HCDR2) comprising any one of SEQ ID NOs: 15-31, 168-173, or 203-206, and a complementarity determining region 3 (HCDR3) comprising any one of SEQ ID NOs: 32-52, 174-177, 207, or 208; or

the immunoglobulin light chain variable region polypeptide comprises a complementarity determining region 1 (LCDR1) comprising any one of SEQ ID NOs: 53-67 or 178-182, a complementarity determining region 2 (LCDR2) comprising any one of SEQ ID NOs: 68-79, and a complementarity determining region 3 (LCDR3) comprising any one of SEQ ID NOs: 80-91.

(2) A PD-L1 binding agent comprising an immunoglobulin heavy chain variable region of any one of SEQ ID NOs: 123-143, 184-193, or 209-213, or at least the CDRs thereof; and an immunoglobulin light chain variable region of any one of SEQ ID NOs: 144-164 or 194-202 or at least the CDRs thereof.

(3) A PD-L1 binding agent comprising an immunoglobulin heavy chain variable region polypeptide with an amino acid sequence that is at least 90% identical to any one of SEQ ID NOs: 123-143, 184-193, or 209-213, and an immunoglobulin light chain variable region polypeptide with an amino acid sequence that is at least 90% identical to any one of SEQ ID NOs: 144-164 or 194-202.

(4) The PD-L1 binding agent of any one of aspects 1-3, which comprises the heavy and light chain immunoglobulin polypeptides, or at least the CDRs thereof, of a PD-L1 binding agent of Table 1.

(5) The PD-L1 binding agent of any one of aspects 1-4, wherein the binding agent is an antibody, an antibody conjugate, or an antigen-binding fragment thereof.

(6) The PD-L1 binding agent of aspect 5, wherein the binding agent is an antibody fragment selected from F(ab′)₂, Fab′, Fab, Fv, scFv, dsFv, dAb, and a single chain binding polypeptide.

(7) The PD-L1 binding agent of aspect 5, wherein the binding agent is an antibody.

(8) The PD-L1 binding agent of any of aspects 1-7, further comprising an immunoglobulin Fc region.

(9) The PD-L1 binding agent of aspect 8, further comprising a transforming growth factor beta 1 (TGFβ1) receptor, or a fragment thereof that binds TGFβ1, attached to the Fc region.

(10) The PD-L1 binding agent of any one of aspects 7-9, wherein the antibody is an IgG, IgM, IgA, IgD, or IgE antibody.

(11) The PD-L1 binding agent of any one of aspects 7-9, wherein the antibody is an IgG antibody.

(12) The PD-L1 binding agent of any one of aspects 7-11, wherein the antibody exhibits antibody dependent cell-mediated cytotoxicity (ADCC), antibody-dependent cell-mediated phagocytosis (ADCP), or complement dependent cytotoxicity (CDC).

(13) The PD-L1 binding agent of any of aspects 1-12, wherein the binding agent is part of a bispecific antibody, chimeric antigen receptor, chimeric T cell receptor, or bispecific T-cell engager.

(14) A nucleic acid encoding the heavy chain immunoglobulin polypeptide of the anti-PD-L1 binding agent of any one of aspects 1-13.

(15) A nucleic acid encoding the light chain immunoglobulin polypeptide of the anti-PD-L1 binding agent of any one of aspects 1-14.

(16) A nucleic acid encoding the heavy chain immunoglobulin polypeptide and the light chain immunoglobulin polypeptide of the PD-L1 binding agent of any one of aspects 1-15.

(17) A vector comprising the nucleic acid sequence of any one of aspects 14-16.

(18) An isolated cell comprising the nucleic acid of any one of aspects 14-16, optionally in a vector.

(19) A method of providing a PD-L1 binding agent of any of aspects 1-13, the method comprising expressing in a cell in vitro one or more nucleic acids encoding the immunoglobulin heavy and light chain polypeptides thereof.

(20) A composition comprising the PD-L1 binding agent of any one of aspects 1-13 or nucleic acid of any one of aspects 14-16, optionally in a vector, and a pharmaceutically acceptable carrier.

(21) The PD-L1 binding agent of any one of aspects 1-13 or the composition of aspect 20 for use as a medicament for treating a disease, disorder, or condition in a mammal that is responsive to PD-L1 inhibition or binding.

(22) The PD-L1 binding agent or composition of aspect 21, wherein the disease, disorder, or condition is cancer.

(23) The PD-L1 binding agent of any one of aspects 1-13 or the composition of aspect 19 for use as a medicament for enhancing or reducing or inhibiting an immune response in a mammal.

(24) The PD-L1 binding agent for use according to aspect 23, wherein the immune response is an anti-cancer immune response.

(25) A method for treating a disease, disorder, or condition in a mammal that is responsive to PD-L1 inhibition or binding, the method comprising administering the PD-L1 binding agent of any one of aspects 1-13 or the composition of aspect 20 to the mammal.

(26) The method of aspect 25, wherein the disease, disorder, or condition is cancer.

(27) The method of aspect 25, wherein the disease, disorder, or condition is an autoimmune disorder.

(28) The method of aspect 25, wherein the disease, disorder, or condition is an infection.

(29) A method for enhancing or reducing or inhibiting an immune response in a mammal comprising administering the PD-L1 binding agent of any one of aspects 1-13 or the composition of aspect 20 to the mammal.

(30) The method of aspect 29, wherein the immune response is an anti-cancer immune response.

(31) A method of preparing a PD-L1 binding agent of any of aspects 1-13, the method comprising expressing in a cell a nucleic acid comprising a sequence encoding the immunoglobulin heavy chain variable region polypeptide, and a nucleic acid comprising a sequence encoding the immunoglobulin light chain variable region polypeptide, wherein the sequence encoding the heavy chain variable region polypeptide and the sequence encoding the light chain variable region polypeptide can be part of a single nucleic acid or separate nucleic acids.

(32) A method of delivering a payload to a cell expressing PD-L1, the method comprising administering to the cell, or a mammal comprising the cell, a conjugate comprising a (1) PD-L1 binding agent of any one of aspects 1-13 and a (2) payload to the mammal.

(33) A hybridoma or cell line that expresses a PD-L1 binding agent of any of aspects 1-13.

The following examples further illustrate the invention but, of course, should not be construed as in any way limiting its scope.

### EXAMPLES

### Example 1

This example demonstrates that the binding agents according to embodiments of the invention are effective at binding human PD-L1 (hPD-L1) on a cell surface.

Binding of the binding agents of an embodiment of the invention to human PD-L1 (hPD-L1) on cell surfaces was assessed by incubating the binding agents with JIMT-1 cells that express endogenous hPD-L1. Briefly, the binding agents were diluted serially in FACS buffer (PBS+1% FBS+5 mM EDTA, pH 7.4) at 4° C. Diluted binding agents were applied to JIMT-1 cells (˜1×10⁵-2×10⁵ per well) and incubated at 4° C. Cells were subsequently washed twice to remove unbound binding agents and subsequently incubated with a fluorescent anti-human IgG Fc antibody at 4° C. to detect cell-bound anti-PD-L1 binding agents. After unbound secondary antibody was washed out, cells were resuspended in FACS buffer plus 2% formaldehyde and analyzed by flow cytometry. The intensity of fluorescence, expressed as geometric mean fluorescence intensity, was used to determine anti-PDL1 binding agent binding to hPD-L1 on cell surface.

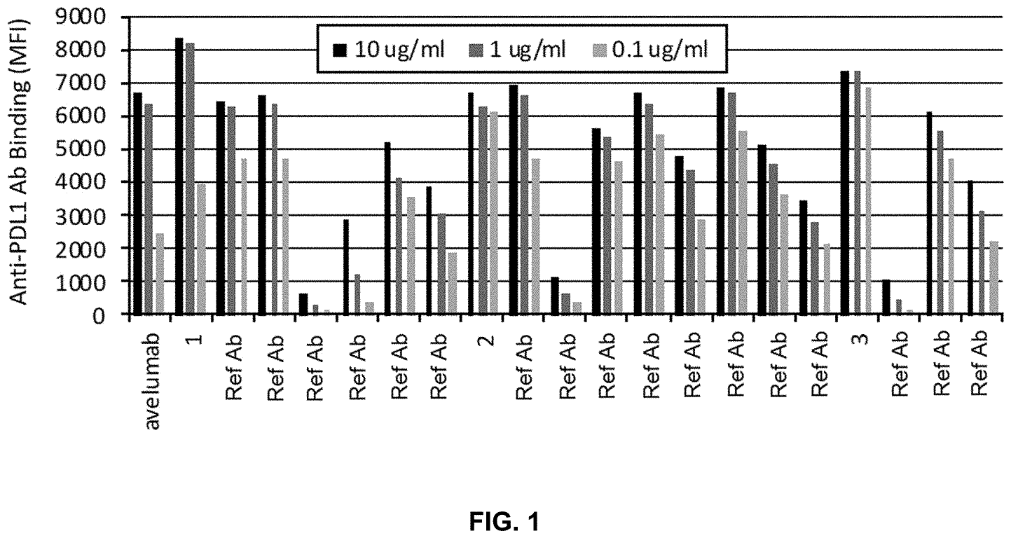

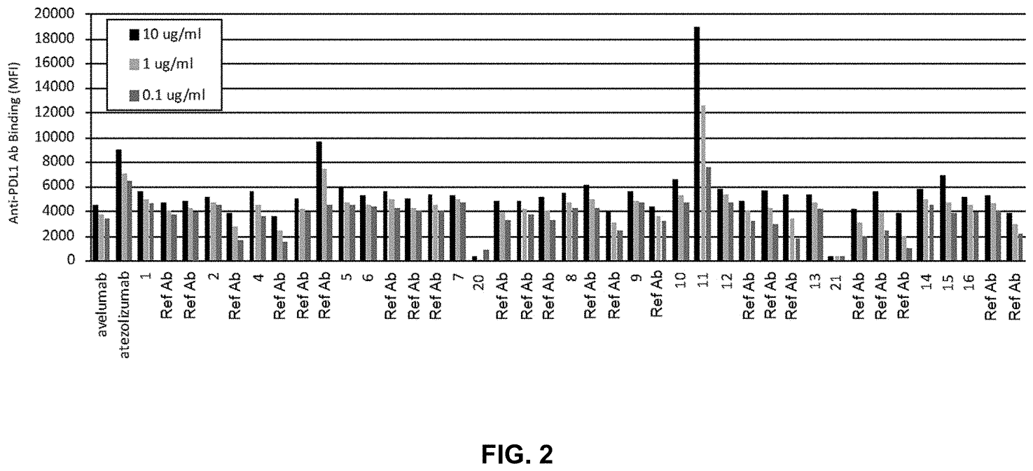

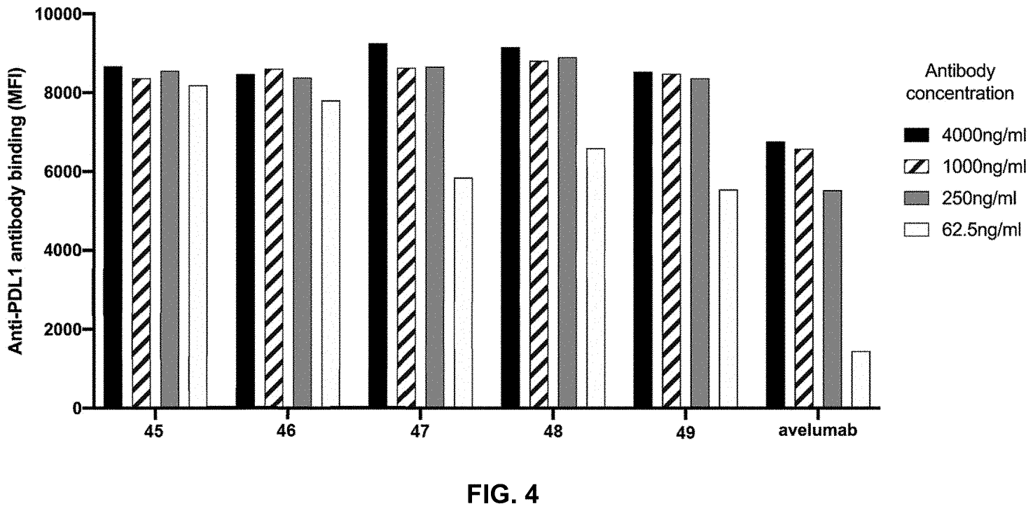

As shown by the data presented in FIGS. 1, 2, and 4, the binding agents according to embodiments of the present invention were successful at binding human PD-L1. The numbers along the x-axis correlate to the numbers of the binding agents according to embodiments of the invention. “Ref Ab” in the figures refers to reference binding agents that are not embodiments of the present invention. The binding agent concentrations are listed in the figure legends.

### Example 2

This example demonstrates that binding agents according to embodiments of the invention were not internalized when exposed to cells with PD-L1 on the cells' surface.

Anti-PDL1 binding agent internalization was measured by estimating their extent of cell surface persistence upon incubation of cells with pre-bound antibodies at 37° C. over a prolonged time course. Briefly, to measure anti-PDL1 antibody surface persistence, JIMT-1 cells were seeded at ˜2×10⁵-4×10⁵ cells per well. On day 1, one set of samples (the “chase” set) was generated by incubating JIMT-1 cells for 60 minutes at 4° C. with anti-PDL1 antibodies at 10 μg/ml in cell culture medium. After the incubation, unbound binding agents were removed by successive washes with culture medium, then cell were incubated at 37° C. in fresh culture medium for 24 hr. On day 2, cells were recovered from the microtiter plates by brief incubation with PBS+10 mM EDTA. The second set of samples (the “no chase” set) was generated on day 2 by incubating JIMT-1 cells with anti-PDL1 binding agents as described above, removing the unbound antibodies and then immediately recovering the cells using PBS+10 mM EDTA.

Recovered JIMT-1 cells were transferred to 96-well microtiter plates, pelleted, and incubated with a fluorescent anti-human IgG Fc antibody in FACS buffer (PBS+1% FBS+5 mM EDTA, pH 7.4) for at least 45 minutes at 4° C. to detect cell-bound anti-PDL1 antibodies. Unbound binding agents were removed by washing cells twice, and cells were resuspended in FACS buffer+2% formaldehyde and analyzed by flow cytometry.

The intensity of fluorescence in the secondary anti-Fc channel, expressed as geometric mean fluorescence intensity, was used to determine anti-PDL1 binding. The ratio in fluorescence intensity of cells incubated with a given anti-PDL1 binding agent followed by a 24 hr chase versus no chase is a measure of the surface persistence of that binding agent. For example, a ratio of 1.0 indicates no loss of surface-bound binding agent over the course of the 24 hr chase, while a ratio <1 indicates that a proportion of binding agent was internalized.

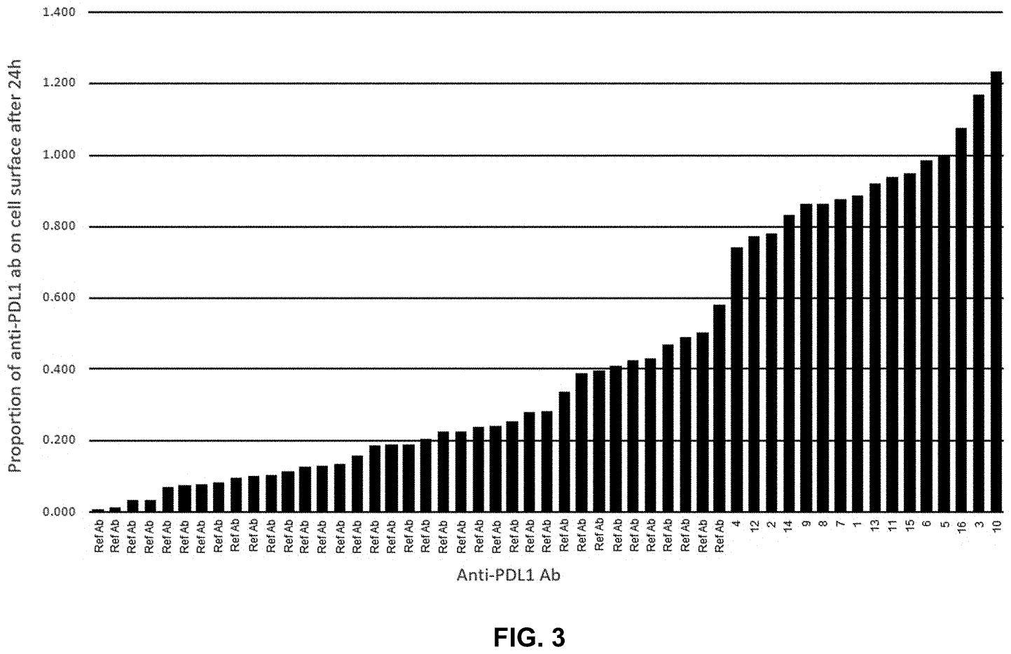

As shown by the data presented in FIG. 3, the binding agents according to embodiments of the present invention were not internalized by the cells. The numbers along the x-axis correlate to the numbers of the binding agents according to embodiments of the invention. “Ref Ab” in the figure refers to reference binding agents that are not embodiments of the present invention.

### Example 3

This example demonstrates that binding agents according to embodiments of the invention inhibit PD-L1/PD-1 binding.

The effects of the binding agents and two commercially available antibodies, avelumab and atezolizumab, on the PD-L1/PD-1 interaction were determined using a reporter assay (Promega). The assay was performed according to manufacturer's instructions. Briefly, cells expressing both PD-L1 and T cell receptor (TCR) activating alphaAPC at the surface were incubated with cells expressing surface recombinant TCR and PD-1 and bearing a luciferase gene that is under the control of the NFAT (nuclear factor of activated T cells) response element.

A trans PD-L1/PD-1 interaction prevents the induction of luciferase expression that occurs through trans alphaAPC-TCR binding, TCR signaling, and NFAT response element activation. Anti-PDL1 antibodies that block the interaction with PD-1 thereby allow TCR signaling and luciferase expression. Luciferase expression is measured using a bio-luminescent substrate and a plate reader set in luminescence detection mode.

Stimulator CHO-K1/PD-L1/alphaAPC cells were suspended into Ham's F-12 medium supplemented with 10% fetal bovine serum (FBS) and plated into wells of six 96-well, clear bottom, white wall plates (Corning). Plates were placed into a 37° C. incubator under 5% CO₂ atmosphere for fourteen hours. Culture medium was removed, and test articles diluted to 2× final concentration in RPMI-1640 medium supplemented with 1% FBS were added to the wells. An equal volume of reporter Jurkat/PD-1/luciferase cells suspended in RPMI-1640+1% FBS was then added to each well. The final concentrations ranged from 4.12 to 27,000 ng/ml in three-fold increments. Avelumab was included as a reference. After eight hours in a 37° C. incubator under 5% CO₂ atmosphere, plates were removed and allowed to equilibrate to room temperature for ten minutes. Wells then received BIOGLOW cell-permeabilizing luciferase substrate (Promega) for ten minutes with gentle agitation.

Luminescence was measured using a Molecular Devices M3 plate reader. Luminescence data were analyzed using GraphPad PRISM v8 for four-parameter sigmoidal dose-response curve fitting and determination of EC50 values. The concentrations were converted to log 10 values for EC50 determinations and for graphing; the “no test article” concentration was converted to (lowest concentration tested)/3 to allow log 10 transformation. Conversion of EC50 values from ng/ml to nM was done using IgG molecular weight data.

PD-L1/PD-1 blockade was demonstrated by the increase in luminescence (RLU=relative luminescence units) induced by increasing concentrations of anti-PDL1 antibodies. Dose-response curves were closely fit using a four-parameter sigmoidal model, consistent with saturable antibody binding.

TABLE 5
Examples of PD-L1/PD-1 blockade EC50 values
(ng/mL, nM) for anti-PDL1 binding agents

| Binding agent | plate | EC50 (ng/ml) | MW (Da) | EC50 (nM) |
|---|---|---|---|---|
| 1 | 1 | 106.5 | 144705 | 0.74 |
| 4 | 1 | 123.6 | 144840 | 0.85 |
| 9 | 1 | 97.3 | 144886 | 0.67 |
| Avelumab | 2 | 67.5 | 143540 | 0.47 |
| 7 | 2 | 106.3 | 145265 | 0.73 |
| 16 | 2 | 180.6 | 144205 | 1.25 |
| Ref Ab | 3 | 290.8 | 144209 | 2.02 |
| 6 | 3 | 105.8 | 144977 | 0.73 |
| 10 | 3 | 67.4 | 144797 | 0.47 |
| 5 | 4 | 54.3 | 144808 | 0.37 |
| Avelumab | 4 | 73.1 | 143540 | 0.51 |
| 14 | 4 | 166.0 | 144329 | 1.15 |
| 3 | 5 | 47.3 | 144447 | 0.33 |
| Ref Ab | 5 | 129.4 | 144437 | 0.9 |
| Atezolizumab | 5 | 57.1 | 144355 | 0.4 |
| 11 | 6 | 53.6 | 144813 | 0.37 |
| 15 | 6 | 163.9 | 145130 | 1.13 |
| Avelumab | 6 | 55.2 | 143540 | 0.38 |

In summary, binding agents according to embodiments of the invention inhibited protein binding, with EC50 values ranging from 47.3 to 180.6 ng/ml (0.33 to 2.02 nM).

The terms “about” and “around,” as used herein to modify a numerical value, indicate a close range surrounding the numerical value. Thus, if “X” is the value, “about X” or “around X” indicates a value of from 0.9X to 1.1X, e.g., from 0.95X to 1.05X or from 0.99X to 1.01X. A reference to “about X” or “around X” specifically indicates at least the values X, 0.95X, 0.96X, 0.97X, 0.98X, 0.99X, 1.01X, 1.02X, 1.03X, 1.04X, and 1.05X. Accordingly, “about X” and “around X” are intended to teach and provide written description support for a claim limitation of, e.g., “0.98X.”

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A programmed death-ligand 1 (PD-L1) binding agent comprising an immunoglobulin heavy chain variable region polypeptide and an immunoglobulin light chain variable region polypeptide, wherein:
the immunoglobulin heavy chain variable region polypeptide comprises a complementarity determining region 1 (HCDR1) comprising any one of SEQ ID NOs: 1-14 and 167, a complementarity determining region 2 (HCDR2) comprising any one of SEQ ID NOs: 15-31, 168-173, and 203-206, and a complementarity determining region 3 (HCDR3) comprising any one of SEQ ID NOs: 32-52, 174-177, 207, and 208; or
the immunoglobulin light chain variable region polypeptide comprises a complementarity determining region 1 (LCDR1) comprising any one of SEQ ID NOs: 53-67 and 178-182, a complementarity determining region 2 (LCDR2) comprising any one of SEQ ID NOs: 68-79, and a complementarity determining region 3 (LCDR3) comprising any one of SEQ ID NOs: 80-91.

2. A PD-L1 binding agent comprising an immunoglobulin heavy chain variable region of any one of SEQ ID NOs: 123-143, 184-193, and 209-213, or at least the complementarity determining regions (CDRs) thereof and an immunoglobulin light chain variable region of any one of SEQ ID NOs: 144-164 and 194-202 or at least the CDRs thereof.

3. A PD-L1 binding agent comprising an immunoglobulin heavy chain variable region polypeptide with an amino acid sequence that is at least 90% identical to any one of SEQ ID NOs: 123-143, 184-193, and 209-213, and an immunoglobulin light chain variable region polypeptide with an amino acid sequence that is at least 90% identical to any one of SEQ ID NOs: 144-164 and 194-202.

4. The PD-L1 binding agent of claim 1, which comprises the heavy and light chain immunoglobulin polypeptides, or at least the complementarity determining regions (CDRs) thereof, of a PD-L1 binding agent of Table 1.

5. The PD-L1 binding agent of claim 1, wherein the binding agent is an antibody, an antibody conjugate, or an antigen-binding fragment thereof.

6. The PD-L1 binding agent of claim 5, wherein the binding agent is an antibody fragment selected from F(ab′)₂, Fab′, Fab, Fv, scFv, dsFv, dAb, and a single chain binding polypeptide.

7. The PD-L1 binding agent of claim 5, wherein the binding agent is an antibody.

8. The PD-L1 binding agent of claim 1, further comprising an immunoglobulin Fc region.

9. The PD-L1 binding agent of claim 8, further comprising a transforming growth factor beta 1 (TGFβ1) receptor, or a fragment thereof that binds TGFβ1, attached to the Fc region.

10. The PD-L1 binding agent of claim 7, wherein the antibody is an IgG, IgM, IgA, IgD, or IgE antibody.

11. The PD-L1 binding agent of claim 7, wherein the antibody is an IgG antibody.

12. The PD-L1 binding agent of claim 7, wherein the antibody exhibits antibody dependent cell-mediated cytotoxicity (ADCC), antibody-dependent cell-mediated phagocytosis (ADCP), or complement dependent cytotoxicity (CDC).

13. The PD-L1 binding agent of claim 1, wherein the binding agent is part of a bispecific antibody, chimeric antigen receptor, chimeric T cell receptor, or bispecific T-cell engager.

14. A nucleic acid encoding the heavy chain immunoglobulin polypeptide of the anti-PD-L1 binding agent of claim 1.

15. A nucleic acid encoding the light chain immunoglobulin polypeptide of the anti-PD-L1 binding agent of claim 1.

16. A nucleic acid encoding the heavy chain immunoglobulin polypeptide and the light chain immunoglobulin polypeptide of the PD-L1 binding agent of claim 1.

17. A vector comprising the nucleic acid sequence encoding the heavy and/or light chains of the anti-PD-L1 binding agent of claim 1.

18. An isolated cell comprising the nucleic acid encoding the heavy and/or light chains of the anti-PD-L1 binding agent of claim 1, optionally in a vector.

19. A method of providing a PD-L1 binding agent of claim 1, the method comprising expressing in a cell in vitro one or more nucleic acids encoding the immunoglobulin heavy and light chain polypeptides thereof.

20. A composition comprising the PD-L1 binding agent of claim 1 or a nucleic acid encoding the heavy and/or light chains of same, optionally in a vector, and a pharmaceutically acceptable carrier.

21.-24. (canceled)

25. A method for treating a disease, disorder, or condition in a mammal that is responsive to PD-L1 inhibition or binding, the method comprising administering the PD-L1 binding agent of claim 1 or conjugate comprising same, or a composition comprising the PD-L1 binding agent or conjugate comprising same, to the mammal.

26. The method of claim 25, wherein the disease, disorder, or condition is cancer.

27. The method of claim 25, wherein the disease, disorder, or condition is an autoimmune disorder.

28. The method of claim 25, wherein the disease, disorder, or condition is an infection.

29. A method for enhancing or inhibiting an immune response in a mammal comprising administering the PD-L1 binding agent of claim 1 or conjugate comprising same, or a composition comprising the PD-L1 binding agent or conjugate comprising same, to the mammal.

30. The method of claim 29, wherein the immune response is an anti-cancer immune response.

31. A method of delivering a payload to a cell expressing PD-L1, the method comprising administering to the cell, or a mammal comprising the cell, a conjugate comprising a PD-L1 binding agent of claim 1 and a payload to the mammal.

32. A hybridoma or cell line that expresses a PD-L1 binding agent of claim 1.
